(19) United States
(12) Patent Application Publication
(10) Pub. No.: US 2022/0064196 A1
(43) Pub. Date: Mar. 3, 2022

(54) EGFR Inhibitors, Compositions and Methods Thereof

(71) Applicant: Betta Pharmaceuticals Co., Ltd., Zhejiang (CN)

(72) Inventors: Xiangyong LIU, Beijing (CN); Jiabing WANG, Beijing (CN); Lieming DING, Zhejiang (CN)

(21) Appl. No.: 17/422,700

(22) PCT Filed: Jan. 14, 2020
(86) PCT No.: PCT/CN2020/071913

§ 371 Date: Jul. 13, 2021

(30) Foreign Application Priority Data
Jan. 17, 2019 (CN) PCT/CN2019/072161

(51) Int. Cl. C07F 9/6561

(52) U.S. Cl. CPC C07F 9/6561

(57) ABSTRACT

The present invention relates to compounds of Formula I, methods of using the compounds as EGFR inhibitors, and pharmaceutical compositions comprising such compounds. The compounds are useful in treating, preventing or ameliorating diseases or disorders such as cancer or infections.

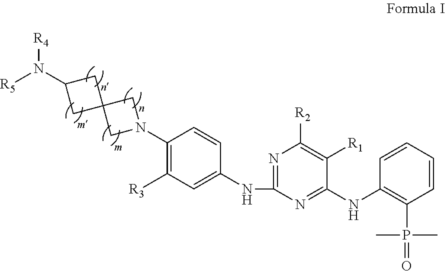

### FIELD OF THE INVENTION

The present application is concerned with pharmaceutically active compounds. The disclosure provides compounds as well as their compositions and methods of use. The compounds inhibit mutant EGFR, including EGFR C797S, and are useful in the treatment of various diseases including infectious diseases and cancers.

### BACKGROUND OF THE INVENTION

Epidermal Growth Factor Receptor (EGFR) is a transmembrane glycoprotein that belongs to ErbB family of tyrosine kinase receptors. Activation of EGFR leads to autophosphorylation of receptor tyrosine kinase that initiates a cascade of downstream signaling pathways involved in regulating cellular proliferation, differentiation, and survival. EGFR is abnormally activated by various mechanisms such as receptor overexpression, mutation, ligand-dependent receptor dimerization, ligand-independent activation and is associated with the development of various human cancers.

EGFR inhibition is for a major cancer therapy. Although the previous generations of EGFR-TKIs have developed rapidly, the problem of drug resistance has also followed with the use of drugs. Most of the drug resistance is the T790M mutation in the ATP pocket. The recently developed third-generation series of irreversible inhibitors have very good inhibitory activity against T790M, but inevitably, the acquired mutation of C797S occurs, such as osimertinib. A high percentage of these treated patients developed a tertiary cystein-797 to serine-790 (C797S) mutation in the EGFR kinase domain. This C797S mutation is thought to induce resistance to all current irreversible EGFR TKIs.

Earlier applications: WO2018108064, WO2018115218 &WO2018181777, disclosed a series of 4^{th} EGFR inhibitors. Accordingly, there is still a need for selective molecules that specifically inhibit EGFR containing C797S mutants useful for the therapeutic and/or prophylactic treatment of cancer. In this invention, applicant discovered potent small molecules that can have activity as 4^{th} generation of EGFR inhibitors, and thus may be useful for therapeutic administration to fight against cancer and/or infectious diseases. These small molecules are expected to be useful as pharmaceuticals with desirable stability, solubility, bioavailability, therapeutic index and toxicity values that are crucial to become efficient medicines to promote human health.

### SUMMARY OF INVENTION

The present invention relates to compounds that are used as EGFR tyrosine kinase inhibitors. These inhibitors are useful in the treatment of cancers and infectious diseases.

The compounds of the invention have the general structures as Formula I. A compound of Formula I, or a stereoisomer, tautomer, pharmaceutically acceptable salt, prodrug, chelate, non-covalent complex, or solvate thereof,

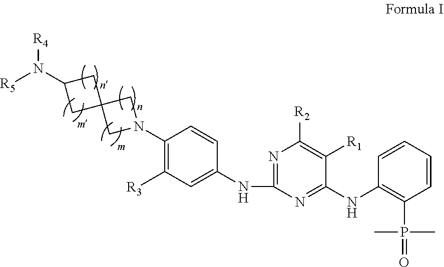

wherein,

R₁ is H, CN, halogen, —C₁₋₆ alkyl or C₁₋₆ alkoxyl;

R₂ is H, halogen, or —C₁₋₆ alkyl; or

R₁ and R₂ together with the atoms to which they are attached form a 5- to 6-membered heteroaryl ring optionally comprising 1, 2 or 3 hetero atoms independently selected from N, S, or O;

R₃ is H, halogen, —C₁₋₆ alkyl, 13 C₁₋₄haloalkyl, —C₃₋₆ carbocyclic ring;

R₄ and R₅ are each independently selected from H, —C₁₋₆ alkyl, —C₁₋₄ alkyl-OH, or —C₃₋₆ carbocyclic ring; or

R₄ and R₅ together with the atoms to which they are attached form a 5- to 6-membered heterocyclic ring optionally substituted with one or more substituents independently selected from —C₁₋₆ alkyl, halogen, or —NR₆R₇;

R₆ and R₇ are each independently selected from H, or —C₁₋₆ alkyl;

m, n, m′, n′ are each independently selected from 1 or 2.

In some embodiments of Formula I, Ri is independently selected from H, F, Cl, —CH₃, —OCH₃ or CN.

In some embodiments of Formula I, R₂ is H.

In some embodiments of Formula I, R₁ and R₂ together with the atoms to which they are

attached form

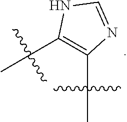 [SMILES: *c1nc[nH]c1*]

In some embodiments of Formula I, R₃ is independently selected from H, CH₃,

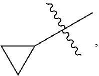 [SMILES: *C1CC1]

### Cl, F, and CF₃.

In some embodiments of Formula I, R₄ and R₅ are independently selected from H, —CH₃, CH₂CH₂OH,

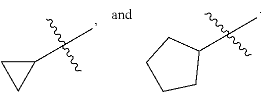

In some embodiments of Formula I, R₄ and R₅ together with the atoms to which they are attached form

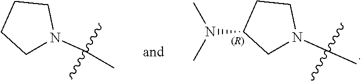

The present invention further provides some preferred technical solutions with regard to compound of Formula (I), compound is:

1) (2-((5-chloro-2-((4-(7-(dimethylamino)-2-azaspiro[3.5]nonan-2-yl)-3-methylphenyl)amino)pyrimidin-4-yl)amino)phenyl)dimethylphosphine oxide;

2) (2-((5-chloro-2-((3-methyl-4-(7-(methylamino)-2-azaspiro[3.5]nonan-2-yl)phenyl)amino)pyrimidin-4-yl)amino)phenyl)dimethylphosphine oxide;

3) (2-((2-((4-(7-amino-2-azaspiro[3.5]nonan-2-yl)-3-methylphenyl)amino)-5-chloropyrimidin-4-yl)amino)phenyl)dimethylphosphine oxide;

4) (2-((5-chloro-2-((4-(7-(cyclopropylamino)-2-azaspiro[3.5]nonan-2-yl)-3-methylphenyl)amino)pyrimidin-4-yl)amino)phenyl)dimethylphosphine oxide;

5) (2-((5-chloro-2-((3-cyclopropyl-4-(7-(dimethylamino)-2-azaspiro[3.5]nonan-2-yl)phenyl)amino)pyrimidin-4-yl)amino)phenyl)dimethylphosphine oxide;

6) (2-((5-chloro-2-((3-chloro-4-(2-(dimethylamino)-7-azaspiro[3.5]nonan-7-yl)phenyl)amino)pyrimidin-4-yl)amino)phenyl)dimethylphosphine oxide;

7) (2-((2-((3-chloro-4-(2-(dimethylamino)-7-azaspiro[3.5]nonan-7-yl)phenyl)amino)pyrimidin-4-yl)amino)phenyl)dimethylphosphine oxide;

8) (2-((2-((4-(2-amino-7-azaspiro[3.5]nonan-7-yl)-3-chlorophenyl)amino)-5-chloropyrimidin-4-yl)amino)phenyl)dimethylphosphine oxide;

9) (2-((2-((4-(2-amino-7-azaspiro[3.5]nonan-7-yl)-3-chlorophenyl)amino)pyrimidin-4-yl)amino)phenyl)dimethylphosphine oxide;

10) (2-((5-chloro-2-((3-chloro-4-(7-(dimethylamino)-2-azaspiro[3.5]nonan-2-yl)phenyl)amino)pyrimidin-4-yl)amino)phenyl)dimethylphosphine oxide;

11) (2-((2-((4-(7-amino-2-azaspiro[3.5]nonan-2-yl)-3-chlorophenyl)amino)-5-chloropyrimidin-4-yl)amino)phenyl)dimethylphosphine oxide;

12) (2-((5-chloro-2-((3-chloro-4-(7-((2-hydroxyethyl)amino)-2-azaspiro[3.5]nonan-2-yl)phenyl)amino)pyrimidin-4-yl)amino)phenyl)dimethylphosphine oxide;

13) (2-((5-chloro-2-((4-(7-(dimethylamino)-2-azaspiro[3.5]nonan-2-yl)phenyl)amino)pyrimidin-4-yl)amino)phenyl)dimethylphosphine oxide;

14) (2-((2-((4-(2-amino-7-azaspiro[3.5]nonan-7-yl)-3-methylphenyl)amino)-5-chloropyrimidin-4-yl)amino)phenyl)dimethylphosphine oxide;

15) (2-((5-chloro-2-((4-(2-(dimethylamino)-7-azaspiro[3.5]nonan-7-yl)-3-methylphenyl)amino)pyrimidin-4-yl)amino)phenyl)dimethylphosphine oxide;

16) (2-((2-((4-(2-amino-7-azaspiro[3.5]nonan-7-yl)phenyl)amino)-5-chloropyrimidin-4-yl)amino)phenyl)dimethylphosphine oxide hydrochloride;

17) (2-((4-chloro-2-((4-(2-(dimethylamino)-7-azaspiro[3.5]nonan-7-yl)phenyl)amino)pyrimidin-4-yl)amino)phenyl)dimethylphosphine oxide;

18) (2-((2-((4-(2-amino-7-azaspiro[3.5]nonan-7-yl)-3-(trifluoromethyl)phenyl)amino)-5-chloropyrimidin-4-yl)amino)phenyl)dimethylphosphine oxide;

19) (2-((5-chloro-2-((4-(2-(dimethylamino)-7-azaspiro[3.5]nonan-7-yl)-3-(trifluoromethyl)phenyl)amino)pyrimidin-4-yl)amino)phenyl)dimethylphosphine oxide;

20) (2-((5-chloro-2-((3-chloro-4-(2-(methylamino)-7-azaspiro[3.5]nonan-7-yl)phenyl)amino)pyrimidin-4-yl)amino)phenyl)dimethylphosphine oxide;

21) (2-((2-((4-(2-amino-7-azaspiro[3.5]nonan-7-yl)-3-methylphenyl)amino)-9H-purin-6-yl)amino)phenyl)dimethylphosphine oxide;

22) (2-((2-((4-(6-amino-2-azaspiro[3.3]heptan-2-yl)-3-methylphenyl)amino)-5-methylpyrimidin-4-yl)amino)phenyl)dimethylphosphine oxide;

23) (2-((2-((4-(6-amino-2-azaspiro[3.3]heptan-2-yl)-3-methylphenyl)amino)-5-methoxypyrimidin-4-yl)amino)phenyl)dimethylphosphine oxide;

24) (2-((2-((4-(6-amino-2-azaspiro[3.3]heptan-2-yl)-3-methylphenyl)amino)-5-fluoropyrimidin-4-yl)amino)phenyl)dimethylphosphine oxide;

25) (2-((2-((4-(6-amino-2-azaspiro[3.3]heptan-2-yl)-3-methylphenyl)amino)-5-chloropyrimidin-4-yl)amino)phenyl)dimethylphosphine oxide;

26) (2-((2-((4-(6-amino-2-azaspiro[3.3]heptan-2-yl)-3-chlorophenyl)amino)-5-chloropyrimidin-4-yl)amino)phenyl)dimethylphosphine oxide;

27) (2-((2-((4-(2-amino-7-azaspiro[3.5]nonan-7-yl)-3-methylphenyl)amino)-5-methoxypyrimidin-4-yl)amino)phenyl)dimethylphosphine oxide;

28) 2-((4-(2-amino-7-azaspiro[3.5]nonan-7-yl)-3-methylphenyl)amino)-4-((2-(dimethylphosphoryl)phenyl)amino)pyrimidine-5-carbonitrile;

29) (2-((2-((4-(2-amino-7-azaspiro[3.5]nonan-7-yl)-3-fluorophenyl)amino)-5-chloropyrimidin-4-yl)amino)phenyl)dimethylphosphine oxide;

30) (2-((5-chloro-2-((4-(2-(dimethylamino)-7-azaspiro[3.5]nonan-7-yl)-3-fluorophenyl)amino)pyrimidin-4-yl)amino)phenyl)dimethylphosphine oxide;

31) (2-((5-chloro-2-((3-chloro-4-(2-(cyclopentylamino)-7-azaspiro[3.5]nonan-7-yl)phenyl)amino)pyrimidin-4-yl)amino)phenyl)dimethylphosphine oxide;

32) (2-((5-chloro-2-((3-chloro-4-(2-((2-hydroxyethyl)amino)-7-azaspiro[3.5]nonan-7-yl)phenyl)amino)pyrimidin-4-yl)amino)phenyl)dimethylphosphine oxide;

33) (2-((5-chloro-2-((3-chloro-4-(2-(pyrrolidin-1-yl)-7-azaspiro[3.5]nonan-7-yl)phenyl)amino)pyrimidin-4-yl)amino)phenyl)dimethylphosphine oxide;

34) (R)-(2-((5-chloro-2-((3-chloro-4-(2-(3-(dimethylamino)pyrrolidin-1-yl)-7-azaspiro[3.5]nonan-7-yl)phenyl)amino)pyrimidin-4-yl)amino)phenyl)dimethylphosphine oxide.

The present invention also provides a pharmaceutical composition comprising a compound of any one of the present invention, or a pharmaceutically acceptable salt or a stereoisomer thereof, and at least one pharmaceutically acceptable carrier or excipient.

The present invention additionally provided a method of inhibiting mutant EGFR, including EGFR C797S, said method comprising administering to a patient a compound of any one of the present invention or a pharmaceutically acceptable salt or a stereoisomer thereof.

The present invention further provides a method of treating an EGFR-driven cancer, said method comprising administering to a patient in need thereof a therapeutically effective amount of a compound of any one of the present invention, or a pharmaceutically acceptable salt or a stereoisomer thereof.

In some embodiments, the EGFR-driven cancer is characterized by the presence of one or more mutations selected from, but not limited to (i) C797S, (ii) both L858R and C797S, (iii) both C797S and T790M, (iv) L858R, T790M, and C797S, or (v) De119, T790M and C797S.

In some embodiments, the EGFR-driven cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

The present invention provided a method of inhibiting mutant EGFR in a patient, said method comprising administering to the patient in need thereof a therapeutically effective amount of a compound of the present invention, or a pharmaceutically acceptable salt or a stereoisomer thereof.

The present invention also provides a use of the present compound or its pharmaceutical composition for the preparation of a medicament.

In some embodiments, wherein the medicament is used for the treatment or prevention of cancer.

In some embodiments, wherein the cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

In some embodiments, wherein the medicament is used as an inhibitor of EGFR mutants including, but not limited to EGFR C797S.

In some embodiments, the EGFR-driven cancer is non-small-cell lung cancer (NSCLC).

The general chemical terms used in the formula above have their usual meanings. For example, the term “halogen”, as used herein, unless otherwise indicated, means fluoro, chloro, bromo or iodo. The preferred halogen groups include F, Cl and Br.

As used herein, unless otherwise indicated, alkyl includes saturated monovalent hydrocarbon radicals having straight, branched or cyclic moieties. For example, alkyl radicals include methyl, ethyl, propyl, isopropyl, cyclopropyl, n-butyl, isobutyl, sec-butyl, t-butyl, cyclobutyl, n-pentyl, 3-(2-methyl) butyl, 2-pentyl, 2-methylbutyl, neopentyl, cyclopentyl, n-hexyl, 2-hexyl, 2-methylpentyl and cyclohexyl. Similarly, C₁₋₈, as in C₁₋₈alkyl is defined to identify the group as having 1, 2, 3, 4, 5, 6, 7 or 8 carbon atoms in a linear or branched arrangement.

Alkoxy radicals are oxygen ethers formed from the previously described straight, branched chain or cyclic alkyl groups.

The term “aryl”, as used herein, unless otherwise indicated, refers to an unsubstituted or substituted mono- or polycyclic ring system containing carbon ring atoms. The preferred aryls are mono cyclic or bicyclic 6-10 membered aromatic ring systems. Phenyl and naphthyl are preferred aryls. The most preferred aryl is phenyl.

The term “heteroaryl”, as used herein, unless otherwise indicated, represents an unsubstituted or substituted stable five or six membered monocyclic aromatic ring system or an unsubstituted or substituted nine or ten membered benzo-fused heteroaromatic ring system or bicyclic heteroaromatic ring system which consists of carbon atoms and from one to four heteroatoms selected from N, O or S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “cycloalkyl” to a cyclic saturated alkyl chain having from 3 to 12 carbon atoms, for example, cyclopropyl, cyclobutyl, cyclobutyl, cyclobutyl.

The term “substituted” refers to a group in which one or more hydrogen atoms are each independently replaced with the same or different substituent(s). Typical substituents include, but are not limited to, halogen (F, Cl, Br or I), C₁₋₈ alkyl, C₃₋₁₂ cycloalkyl, —OR¹, SR¹, ═O, ═S, —C(O)R¹, —C(S)R¹, ═NR¹, —C(O)OR¹, —C(S)OR¹, —NR¹R², —C(O)NR¹R², cyano, nitro, —S(O)₂R¹, —OS(O₂)OR¹, —O(O)₂R¹, —OP(O)(OR¹)(OR²); wherein R¹ and R² is independently selected from —H, lower alkyl, lower haloalkyl. In some embodiments, the substituent(s) is independently selected from the group consisting of —F, —Cl, —Br, —I, —OH, trifluoromethoxy, ethoxy, propyloxy, iso-propyloxy, n-butyloxy, isobutyloxy, t-butyloxy, —SCH₃, —SC₂H₅, formaldehyde group, C(OCH₃), cyano, nitro, CF₃, —OCF₃, amino, dimethylamino, methyl thio, sulfonyl and acetyl.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

Examples of substituted alkyl group include, but not limited to, 2-aminoethyl, 2-hydroxyethyl, pentachloroethyl, trifluoromethyl, methoxymethyl, pentafluoroethyl and piperazinylmethyl.

Examples of substituted alkoxy groups include, but not limited to, aminomethoxy, trifluoromethoxy, 2-diethylaminoethoxy, 2-ethoxycarbonylethoxy, 3-hydroxypropoxy.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I are shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula I exists, the present invention includes any possible tautomers and pharmaceutically acceptable salts thereof, and mixtures thereof, except where specifically stated otherwise.

When the compound of Formula I and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound, or a pharmaceutically acceptable salt, of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg,100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5wt % to about 10wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, colon cancer, rectal cancer, mantle cell lymphoma, multiple myeloma, breast cancer, prostate cancer, glioblastoma, squamous cell esophageal cancer, liposarcoma, T-cell lymphoma melanoma, pancreatic cancer, glioblastoma or lung cancer, may be effectively treated by the administration of from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy, the subject disposition to the disease, and the judgment of the treating physician.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit EGFR C797S according to at least one assay described herein.

### EXAMPLES

It is to be understood that the foregoing general description and the following detailed description are exemplary and explanatory only and are not restrictive of any subject matter claimed. All parts and percentages are by weight and all temperatures are degrees Celsius, unless explicitly stated otherwise. The compounds described herein can be obtained from commercial sources or synthesized by conventional methods as shown below using commercially available starting materials and reagents.

The following abbreviations have been used in the examples:

AcOH: Acetic acid;

DCM: Dichloromethane;

DIBAL-H: Diisobutylaluminium hydride;

DIEA: N,N-Diisopropylethylamine;

DMF: Dimethylformamide;

DMSO: Dimethyl sulfoxide;

EDTA: Ethylenediaminetetraacetic acid;

EtOAc: Ethyl acetate

HEPES: 4-(2-hydroxyethyl)-1-piperazineethanesulfonic acid;

LCMS: Liquid chromatographymass spectrometry;

h or hrs: hour or hours;

Pd(OH)₂/C: Palladium hydroxide on activated charcoal(Pearlman's catalysts)

MeOH: Methanol;

min: minute;

rt or R.T: room temperature;

TFA: Trifluoroacetic acid;

THF: Tetrahydrofuran;

TLC: Preparative thin layer chromatography;

t-BuONa: Sodium tert-butoxide

t-BuXPhos Pd 2rd: Methanesulfonato(2-di-t-butylphosphino-2′,4′,6′-tri-i-propyl-1,1′-biphenyl)(2′-amino-1,1′-biphenyl-2-y0palladium(II)

### Example 1

Synthesis of Compound 1

### (2-((5-chloro-2-((4-(7-(dimethylamino)-2-azaspiro[3.5]nonan-2-yl)-3-methylphenyl)amino)pyrimidin-4-yl)amino)phenyl)dimethylphosphine oxide

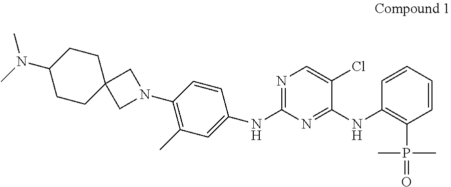

Step 1: Synthesis of 2-azaspiro[3.5]nonan-7-one trifluoroacetate

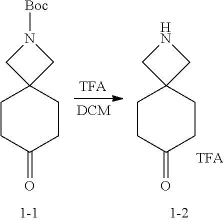 [SMILES: CC(C)(C)OC(=O)N1CC2(CCC(=O)CC2)C1 ; O=C1CCC2(CC1)CNC2]

To a stirred solution of tert-butyl 7-oxo-2-azaspiro[3.5]nonane-2-carboxylate (2.0 g) in DCM (30 mL) was added TFA (10 mL). The reaction mixture was stirred at room temperature for 2 h. After completion of the reaction (monitored by TLC), the reaction mixture was evaporated under reduced pressure to obtain 2-azaspiro[3.5]nonan-7-one trifluoroacetate (3.0 g) as a yellow oil.

Step 2: Synthesis of 2-(2-methyl-4-nitrophenyl)-2-azaspiro[3.5]nonan-7-one

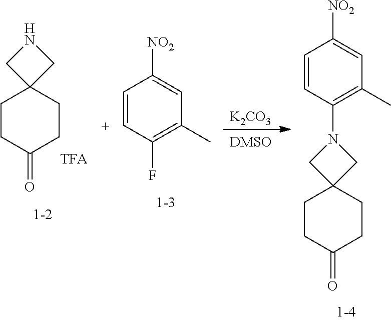

To a solution of 2-azaspiro[3.5]nonan-7-one trifluoroacetic acid salt (3.0 g) and 1-fluoro-2-methyl-4-nitrobenzene (1.5 g) dissolved in DMSO (30 mL) was added K₂CO₃ (1.5 g). The reaction mixture was stirred at 90° C. overnight. The reaction mixture was cooled down to room temperature and diluted with EtOAc (150 mL). The resulting solution was washed with water and NaCl saturated aqueous solution. The mixture was dried over anhydrous sodium sulfate and concentrated under vacuum. The residue was purified by column chromatography over silica gel with ethyl acetate/petroleum ether (1:3). This obtained 1.15 g of 2-(2-methyl-4-nitrophenyl)-2-azaspiro[3.5]nonan-7-one as a yellow solid. MS: 275 [M+H]⁺.

Step 3:Synthesis of 2-(4-amino-2-methylphenyl)-2-azaspiro[3.5]nonan-7-one

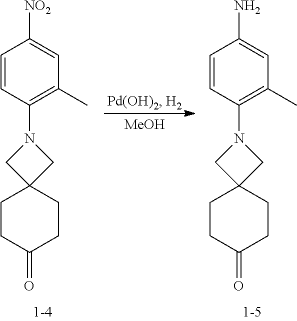 [SMILES: "MeOH" ; Cc1cc([N+](=O)[O-])ccc1N1CC2(CCC(=O)CC2)C1 ; Cc1cc(N)ccc1N1CC2(CCC(=O)CC2)C1]

To a solution of 2-(2-methyl-4-nitrophenyl)-2-azaspiro[3.5]nonan-7-one (1.15 g, 4.19 mmol) dissolved in MeOH (30 mL) was added Palladium Hydroxide 20% on Carbon (1 g, 50% water). A balloon of H₂ gas was connected via a needle to the reaction mixture which was stirred at room temperature for 5 h. The solution was filtered through diatomite to remove the Pd(OH)₂/C. The solution was evaporated to give 800 mg of 2-(4-amino-2-methylphenyl)-2-azaspiro[3.5]nonan-7-one. MS: 245 [M+H]⁺.

Step 4: Synthesis of 2-((4(5-chloro-4-((2-(dimethylphosphoryl)phenyl)amino)pyrimidin-2-yl) amino)-2-methylphenyl)-2-azaspiro[3.5]nonan-7-one

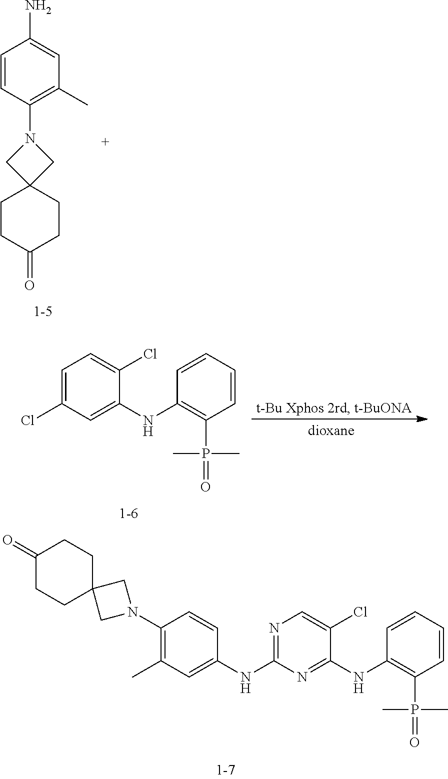 [SMILES: Cc1cc(N)ccc1N1CC2(CCC(=O)CC2)C1 ; CP(C)(=O)c1ccccc1Nc1cc(Cl)ccc1Cl ; Cc1cc(Nc2ncc(Cl)c(Nc3ccccc3P(C)(C)=O)n2)ccc1N1CC2(CCC(=O)CC2)C1]

To a solution of 2-(4-amino-2-methylphenyl)-2-azaspiro[3.5]nonan-7-one (480 mg) and (2-((2,5-dichloropyrimidin-4-yl)amino)phenyl)dimethylphosphine oxide (930 mg) dissolved in dioxane (10 mL) was added t-BuONa (378 mg) and t-BuXPhos Pd 2rd (162 mg). The solution was purged with N₂ and stirred at 130° C. for 3 h by Microwave. The reaction mixture was cooled down to room temperature and diluted with DCM (50 mL). The resulting solution was washed with water and NaCl saturated aqueous solution. The mixture was dried over anhydrous magnesium sulfate and concentrated under vacuum. The residue was purified by column chromatography over silica gel with DCM/MeOH (15:1). This obtained 230 mg of 2-(4-((5-chloro-4-((2-(dimethylphosphoryl)phenyl)amino)pyrimidin-2-yl)amino)-2-methylphenyl)-2-azaspiro[3.5]nonan-7-one as a brown solid. MS: 524 [M+H]⁺.

Step 5: Synthesis of (2-((5-chloro-2-((4-(7-(dimethylamino)-2-azaspiro[3.5]nonan-2-yl)-3-methylphenyl)amino)pyrimidin-4-yl)amino)phenyl)dimethylphosphine oxide

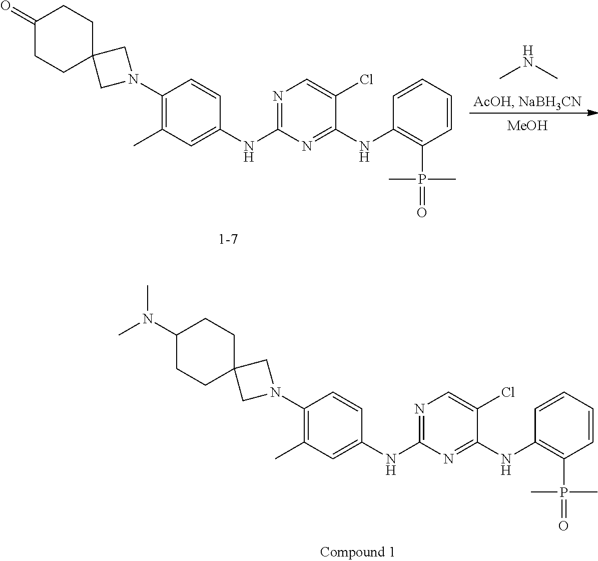

To a solution of 2-(4-4-((5-chloro-4-((2-(dimethylphosphoryl)phenyl)amino)pyrimidin-2-yl)amino)-2-methylphenyl)-2-azaspiro[3.5]nonan-7-one (55 mg) in MeOH (4 mL) was added dimethylamine (0.1 mL, 2N in THF) and AcOH (1 drop). The mixture was stirred at room temperature. After 1 h, sodium cyanoborohydride (13 mg) was added and the mixture was further stirred at room temperature for 4 h. After completion of the reaction (monitored by TLC), the reaction mixture was diluted with DCM (50 mL). The resulting solution was washed with 10% NaHCO₃ aqueous solution and NaCl saturated aqueous solution. The mixture was dried over anhydrous magnesium sulfate and concentrated under vacuum. The residue was purified by column chromatography over silica gel with DCM/MeOH (8:1). This obtained 25 mg of compound (2-((5-chloro-2-((4-(7-(dimethylamino)-2-azaspiro[3.5]nonan-2-yl)-3-methylphenyl)amino)pyrimidin-4-yl)amino)phenyl)dimethylphosphine oxide. MS of Compound 1: 553 [M+H]⁺.

¹H NMR (500 MHz, Methanol-d4): δ 8.46 (s, 1H), 8.01 (s, 1H), 7.59 (dd, J=14.2, 7.7 Hz, 1H), 7.50 (t, J=8.0 Hz, 1H), 7.23 (t, J=7.7 Hz, 1H), 7.18 (d, J=7.2Hz, 2H), 6.48 (d, J=8.2Hz, 1H), 3.67 (s, 2H), 3.58 (s, 2H), 3.19 (t, J=14.2Hz, 1H), 2.84 (s, 6H), 2.18 (d, J=13.1 Hz, 2H), 2.15 (s, 3H), 2.06-2.01 (m, 2H), 1.84 (d, J=15.0 Hz, 6H), 1.63 (dt, J=33.1, 12.9 Hz, 4H).

### Example 2

### Synthesis of Compound 2

### (2-((5-chloro-2-((3-methyl-4-(7-(methylamino)-2-azaspiro[3.5]nonan-2-yl)phenyl)amino)pyrimidin-4-yl)amino)phenyl)dimethylphosphine oxide

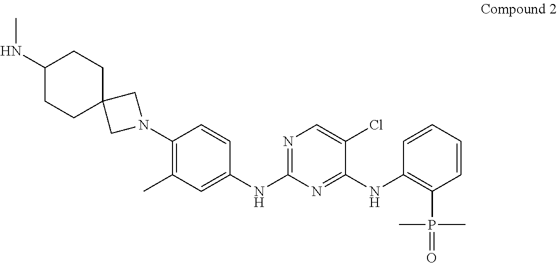 [SMILES: CNC1CCC2(CC1)CN(c1ccc(Nc3ncc(Cl)c(Nc4ccccc4P(C)(C)=O)n3)cc1C)C2]

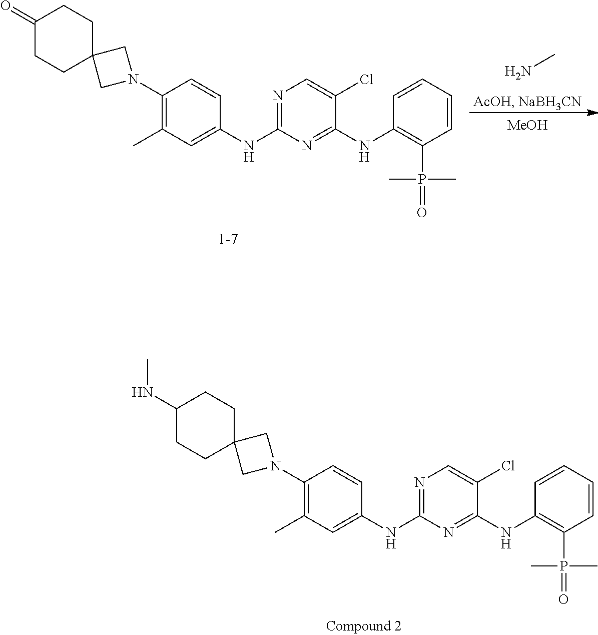

To a solution of 2-(4-((5-chloro-4-((2-(dimethylphosphoryl)phenyl)amino)pyrimidin-2-yl)amino)-2-methylphenyl)-2-azaspiro[3.5]nonan-7-one (60 mg) in MeOH (4 mL) was added methanamine (0.5 mL, 2N in THF) and AcOH (2 drop). The mixture was stirred at room temperature. After 1 h, sodium cyanoborohydride (30 mg) was added and the mixture was further stirred at room temperature for 1 h. After completion of the reaction (monitored by TLC), the reaction mixture was diluted with DCM (30 mL). The resulting solution was washed with 10% NaHCO₃ aqueous solution and NaCl saturated aqueous solution. The mixture was dried over anhydrous magnesium sulfate and concentrated under vacuum. The residue was purified by column chromatography over silica gel with DCM/MeOH (8:1). This obtained 25 mg of compound 2 (2-((5-chloro-2-((3-methyl-4-(7-(methylamino)-2-azaspiro[3.5]nonan-2-yl)phenyl)amino)pyrimidin-4-yl)amino)phenyl)dimethylphosphine oxide. MS: 539 [M+H]⁺.

### Example 3

### Synthesis of Compound 3

### (2-((2-((4-(7-amino-2-azaspiro[3.5]nonan-2-yl)-3-methylphenyl)amino)-5-chloropyrimidin-4-yl)amino)phenyl)dimethylphosphine oxide

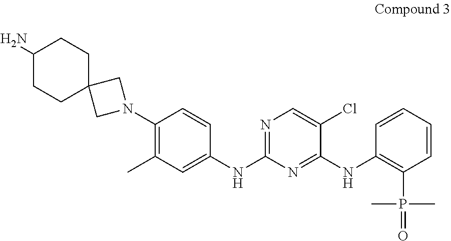 [SMILES: Cc1cc(Nc2ncc(Cl)c(Nc3ccccc3P(C)(C)=O)n2)ccc1N1CC2(CCC(N)CC2)C1]

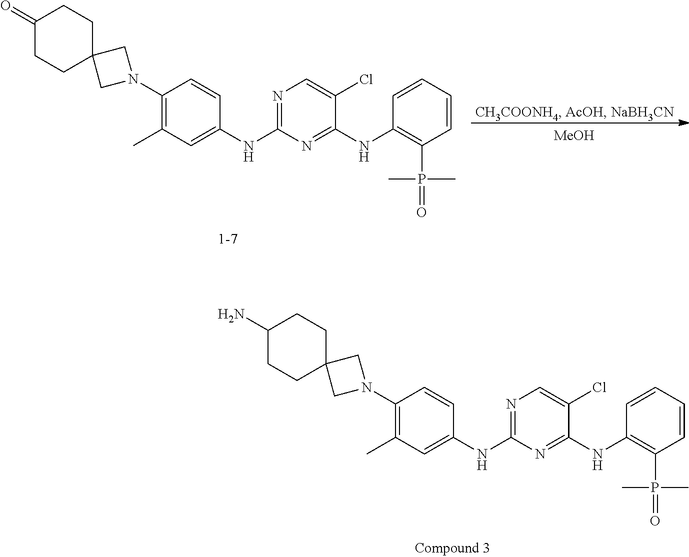 [SMILES: "MeOH" ; Cc1cc(Nc2ncc(Cl)c(Nc3ccccc3P(C)(C)=O)n2)ccc1N1CC2(CCC(=O)CC2)C1 ; Cc1cc(Nc2ncc(Cl)c(Nc3ccccc3P(C)(C)=O)n2)ccc1N1CC2(CCC(N)CC2)C1]

To a solution of 2-(4-((5-chloro-4-((2-(dimethylphosphoryl)phenyl)amino)pyrimidin-2-yl)amino)-2-methylphenyl)-2-azaspiro[3.5]nonan-7-one (70 mg) in MeOH (4 mL) was added ammonium acetate (51 mg) and AcOH (2 drop). The mixture was stirred at room temperature. After 1 h, sodium cyanoborohydride (25 mg) was added and the mixture was further stirred at room temperature for 1 h. After completion of the reaction (monitored by TLC), the reaction mixture was diluted with DCM (30 mL). The resulting solution was washed with 10% NaHCO₃ aqueous solution and NaCl saturated aqueous solution. The mixture was dried over anhydrous magnesium sulfate and concentrated under vacuum. The residue was purified by column chromatography over silica gel with DCM/MeOH (8:1). This obtained 29 mg of compound 3 (2-((2-((4-(7-amino-2-azaspiro[3.5]nonan-2-yl)-3-methylphenyl)amino)-5-chloropyrimidin-4-yl)amino)phenyl)dimethylphosphine oxide. MS: 525 [M+H]⁺

### Example 4

### Synthesis of Compound 4

### (2-((5-chloro-2-((4-(7-(cyclopropylamino)-2-azaspiro[3.5]nonan-2-yl)-3-methylphenyl)amino)pyrimidin-4-yl)amino)phenyl)dimethylphosphine oxide

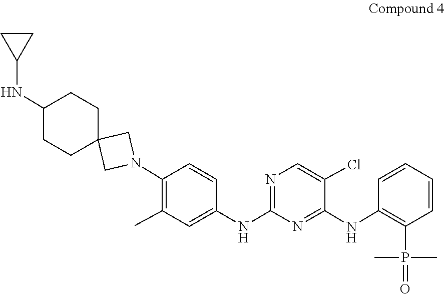 [SMILES: Cc1cc(Nc2ncc(Cl)c(Nc3ccccc3P(C)(C)=O)n2)ccc1N1CC2(CCC(NC3CC3)CC2)C1]

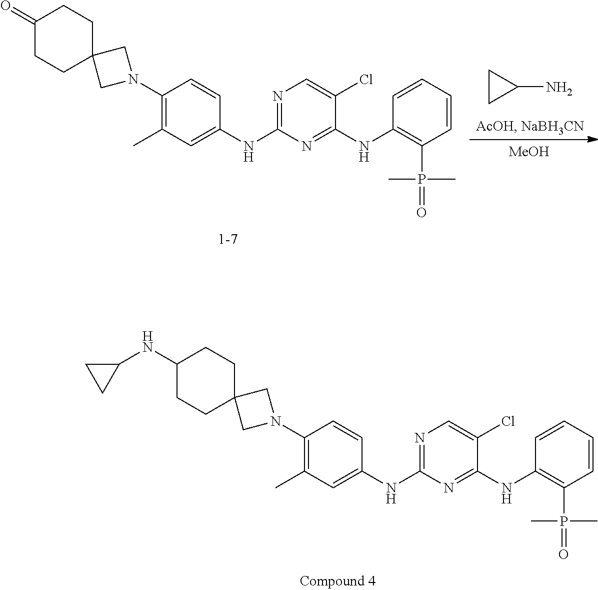

To a solution of 2-(4-((5-chloro-4-((2-(dimethylphosphoryl)phenyl)amino)pyrimidin-2-yl)amino)-2-methylphenyl)-2-azaspiro[3.5]nonan-7-one (80 mg) in MeOH (4 mL) was added ammonium acetate (44 mg) and AcOH (2 drop). The mixture was stirred at room temperature. After 1 h, sodium cyanoborohydride (28 mg) was added and the mixture was further stirred at room temperature for 1 h. After completion of the reaction (monitored by TLC), the reaction mixture was diluted with DCM (50 mL). The resulting solution was washed with 10% NaHCO₃ aqueous solution and NaCl saturated aqueous solution. The mixture was dried over anhydrous magnesium sulfate and concentrated under vacuum. The residue was purified by column chromatography over silica gel with DCM/MeOH (8:1). This obtained 31 mg of compound 4 (2-((5-chloro-2-((4-(7-(cyclopropylamino)-2-azaspiro [3.5]nonan-2-yl)-3-methylphenyl)amino)pyrimidin-4-yl)amino)phenyl)dimethylphosphine oxide. MS: 565 [M+H]⁺

¹H NMR (500 MHz, Methanol-d₄) δ 8.39 (s, 1H), 8.04 (s, 1H), 7.67 (dd, J=14.1, 7.7 Hz, 1H), 7.54 (t, J=7.8 Hz, 1H), 7.36 (t, J=7.7 Hz, 1H), 7.14 (d, J=7.2Hz, 2H), 6.65 (d, J=8.5 Hz, 1H), 3.82 (s, 2H), 3.75 (s, 2H), 3.25 (d, J=11.5 Hz, 1H), 2.81-2.75 (m, 1H), 2.23-2.14 (m, 7H), 1.87 (d, J=13.5 Hz, 6H), 1.71 (t, J=12.0 Hz, 2H), 1.50 (q, J=11.7 Hz, 2H), 0.95 (d, J=7.1 Hz, 2H), 0.86 (d, J=3.5 Hz, 2H).

### Example 5

### Synthesis of Compound 5

### (2-((5-chloro-2-((3-cyclopropyl-4-(7-(dimethylamino)-2-azaspiro[3.5]nonan-2-yl)phenyl)amino)pyrimidin-4-yl)amino)phenyl)dimethylphosphine oxide

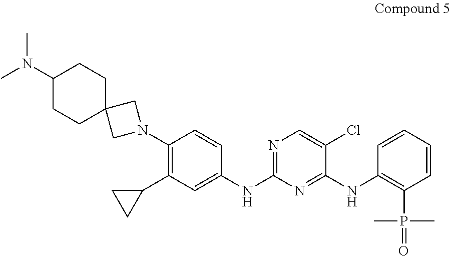

Step 1: Synthesis of 2-cyclopropyl-1-fluoro-4-nitrobenzene

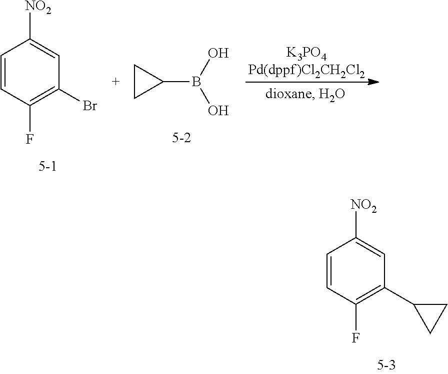 [SMILES: O=[N+]([O-])c1ccc(F)c(Br)c1 ; OB(O)C1CC1 ; O=[N+]([O-])c1ccc(F)c(C2CC2)c1]

To a solution of 2-bromo-1-fluoro-4-nitrobenzene (1 g) and cyclopropylboronic acid (0.78 g) dissolved in dioxane:H₂O=4:1 (10 mL) was added K₃PO₄ (2.9 g) and Pd(dppf)Cl₂CH₂Cl₂ (0.37 g). The solution was purged with N2 and stirred at 90° C. overnight. The reaction mixture was cooled down to room temperature and diluted with EtOAc (50 mL). The resulting solution was washed with water and NaCl saturated aqueous solution. The mixture was dried over anhydrous sodium sulfate and concentrated under vacuum. The residue was purified by column chromatography over silica gel with PE/EtOAc (15:1).

Step 2: Synthesis of 2-(2-cyclopropyl-4-nitrophenyl)-2-azaspiro[3.5]nonan-7-one

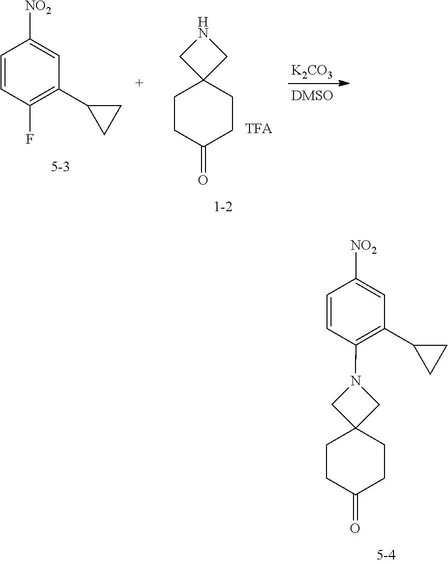

To a solution of 2-cyclopropyl-1-fluoro-4-nitrobenzene (454 mg) and 2-azaspiro[3.5]nonan-7-one trifluoroacetate (0.8 g) dissolved in DMSO (10 mL) was added K₂CO₃ (1.38 g). The reaction mixture was stirred at 90° C. overnight. The reaction mixture was cooled down to room temperature and diluted with EtOAc (50 mL). The resulting solution was washed with water and NaCl saturated aqueous solution. The mixture was dried over anhydrous sodium sulfate and concentrated under vacuum. The residue was purified by column chromatography over silica gel with ethyl acetate/petroleum ether (1:3). This obtained 160 mg of 2-(2-cyclopropyl-4-nitrophenyl)-2-azaspiro[3.5]nonan-7-one as a yellow solid. MS: 301 [M+H]⁺

Step 3: Synthesis of 2-(4-amino-2-cyclopropylphenyl)-2-azaspiro[3.5]nonan-7-one

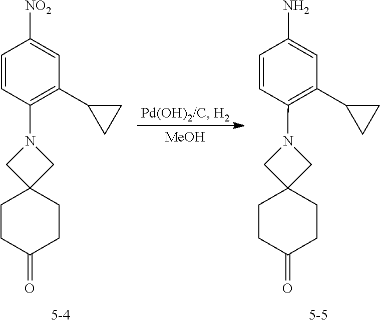 [SMILES: "MeOH" ; O=C1CCC2(CC1)CN(c1ccc([N+](=O)[O-])cc1C1CC1)C2 ; Nc1ccc(N2CC3(CCC(=O)CC3)C2)c(C2CC2)c1]

To a solution of 2-(2-cyclopropyl-4-nitrophenyl)-2-azaspiro[3.5]nonan-7-one (160 mg) dissolved in MeOH (10 mL) was added Palladium Hydroxide 20% on Carbon (100 mg, 50% water). A balloon of H₂ gas was connected via a needle to the reaction mixture which was stirred at room temperature for 3 h. The solution was filtered through diatomite to remove the Pd(OH)₂/C. The solution was evaporated to give 112 mg of 2-(4-amino-2-cyclopropylphenyl)-2-azaspiro[3.5]nonan-7-one. MS: 301 [M+H]⁺.

Step 4: Synthesis of 2-(4-((5-chloro-4-((2-(dimethylphosphoryl)phenyl)amino)pyrimidin-2-yl)amino)-2-cyclopropylphenyl)-2-azaspiro[3.5]nonan-7-one

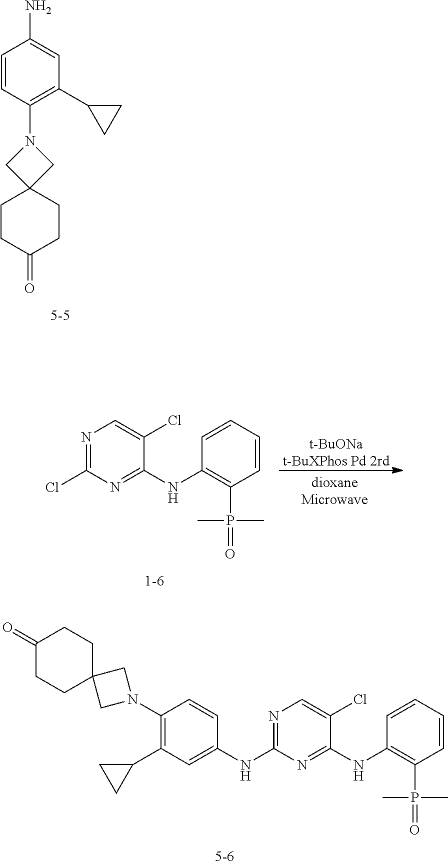 [SMILES: Nc1ccc(N2CC3(CCC(=O)CC3)C2)c(C2CC2)c1 ; CP(C)(=O)c1ccccc1Nc1nc(Cl)ncc1Cl ; CP(C)(=O)c1ccccc1Nc1nc(Nc2ccc(N3CC4(CCC(=O)CC4)C3)c(C3CC3)c2)ncc1Cl]

To a solution of 2-(4-amino-2-cyclopropylphenyl)-2-azaspiro[3.5]nonan-7-one (60 mg) and (2-((2,5-dichloropyrimidin-4-yl)amino)phenyl)dimethylphosphine oxide (105 mg) dissolved in dioxane (3 mL) was added t-BuONa (42 mg) and t-BuXPhos Pd 2rd (162 mg). The solution was purged with N₂ and stirred at 130° C. for 2 h by Microwave. The reaction mixture was cooled down to room temperature and the solids were filtered out. The resulting mixture was concentrated. The residue was purified by column chromatography over silica gel with DCM/MeOH (15:1). This obtained 20 mg of 2-(4-((5-chloro-4-((2-(dimethylphosphoryl)phenyl)amino)pyrimidin-2-yl)amino)-2-cyclopropylphenyl)-2-azaspiro[3.5]nonan-7-one as a brown solid. MS: 550 [M+H]⁺

Step 5: Synthesis of (2-((5-chloro-2-((3-cyclopropyl-4-(7-(dimethylamino)-2-azaspiro[3.5]nonan-2-yl)phenyl)amino)pyrimidin-4-yl)amino)phenyl)dimethylphosphine oxide

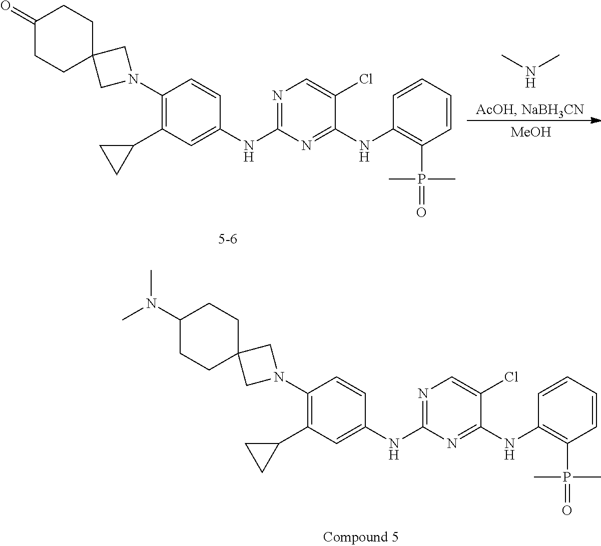

To a solution of 2-(4-((5-chloro-4-((2-(dimethylphosphoryl)phenyl)amino)pyrimidin-2-yl)amino)-2-cyclopropylphenyl)-2-azaspiro[3.5]nonan-7-one (20 mg) in MeOH (4 mL) was added dimethylamine (0.5 mL, 2N in THF) and AcOH (1 drop). The mixture was stirred at room temperature. After 1 h, sodium cyanoborohydride (20 mg) was added and the mixture was further stirred at room temperature for 1 h. After completion of the reaction (monitored by TLC), the reaction mixture was diluted with DCM (15 mL). The resulting solution was washed with 10% NaHCO₃ aqueous solution and NaCl saturated aqueous solution. The mixture was dried over anhydrous magnesium sulfate and concentrated under vacuum. The residue was purified by column chromatography over silica gel with DCM/MeOH (8:1). This obtained 7 mg of (2-((5-chloro-2-((3-cyclopropyl-4-(7-(dimethylamino)-2-azaspiro[3.5]nonan-2-yl)phenyl)amino)pyrimidin-4-yl)amino)phenyl)dimethylphosphine oxide. MS: 579 [M+H]⁺

### Example 6

### Synthesis of Compound 6

### (2-((5-chloro-2-((3-chloro-4-(2-(dimethylamino)-7-azaspiro[3.5]nonan-7-yl)phenyl)amino)pyrimidin-4-yl)amino)phenyl)dimethylphosphine oxide

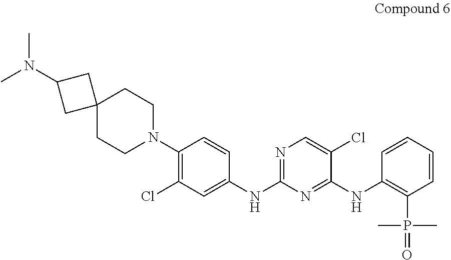

Step 1: Synthesis of tert-butyl (7-(2-chloro-4-nitrophenyl)-7-azaspiro[3.5]nonan-2-yl)carbamate

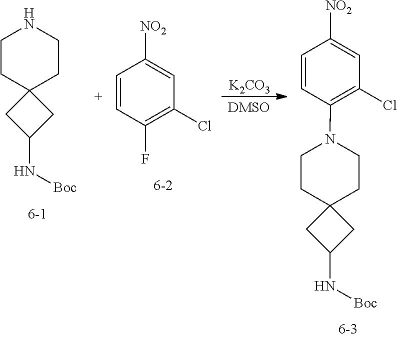

To a solution of tert-butyl (7-azaspiro[3.5]nonan-2-yl)carbamate (550 mg) and 2-chloro-1-fluoro-4-nitrobenzene (400 mg) dissolved in DMSO (10 mL) was added K₂CO₃ (635 mg). The reaction mixture was stirred at 90° C. overnight. The reaction mixture was cooled down to room temperature and diluted with EtOAc (50 mL). The resulting solution was washed with water and NaCl saturated aqueous solution. The mixture was dried over anhydrous sodium sulfate and concentrated under vacuum. The residue was purified by column chromatography over silica gel with ethyl acetate/petroleum ether (1:3). This obtained 830 mg of tert-butyl (7-(2-chloro-4-nitrophenyl)-7-azaspiro[3.5]nonan-2-yl)carbamate as a yellow solid. MS: 396 [M+H]⁺

Step 2: Synthesis of 7-(2-chloro-4-nitrophenyl)-7-azaspiro[3.5]nonan-2-amine trifluoroacetate

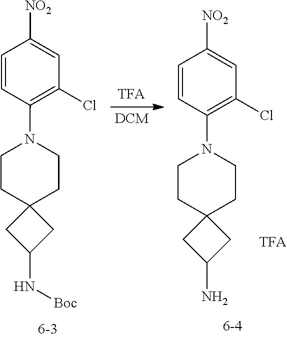 [SMILES: CC(C)(C)OC(=O)NC1CC2(CCN(c3ccc([N+](=O)[O-])cc3Cl)CC2)C1 ; NC1CC2(CCN(c3ccc([N+](=O)[O-])cc3Cl)CC2)C1]

To a stirred solution of (7-(2-chloro-4-nitrophenyl)-7-azaspiro[3.5]nonan-2-yl)carbamate (200 mg) in DCM (10 mL) was added TFA (3 mL). The reaction mixture was stirred at room temperature for 2 h. After completion of the reaction (monitored by TLC), the reaction mixture was evaporated under reduced pressure to obtain 7-(2-chloro-4-nitrophenyl)-7-azaspiro[3.5]nonan-2-amine trifluoroacetate (270 mg, crude) as a yellow oil. MS: 296 [M+H]⁺

Step 3: Synthesis of 7-(2-chloro-4-nitrophenyl)-N,N-dimethyl-7-azaspiro[3.5]nonan-2-amine

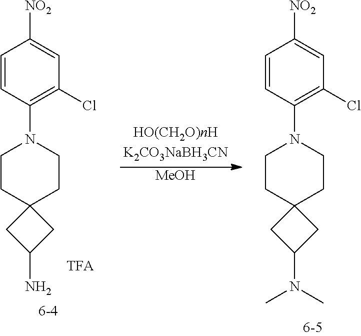 [SMILES: "MeOH" ; NC1CC2(CCN(c3ccc([N+](=O)[O-])cc3Cl)CC2)C1 ; CN(C)C1CC2(CCN(c3ccc([N+](=O)[O-])cc3Cl)CC2)C1]

To a solution of 7-(2-chloro-4-nitrophenyl)-7-azaspiro[3.5]nonan-2-amine trifluoroacetic acid salt (270 mg) in MeOH (10 mL) was added paraformaldehyde (500 mg), K₂CO₃ (800 mg) and sodium cyanoborohydride (200 mg). The mixture is stirred at room temperature for 2 h. After completion of the reaction (monitored by TLC), the reaction mixture was diluted with DCM (50 mL). The resulting solution was washed with H₂O and NaCl saturated aqueous solution. The mixture was dried over anhydrous magnesium sulfate and concentrated under vacuum. The residue was purified by column chromatography over silica gel with DCM/MeOH (15:1). This obtained 120 mg of 7-(2-chloro-4-nitrophenyl)-N,N-dimethyl-7-azaspiro[3.5]nonan-2-amine. MS: 324 [M+H]⁺

Step 4: Synthesis of 7-(4-amino-2-chlorophenyl)-N,N-dimethyl-7-azaspiro[3.5]nonan-2-amine

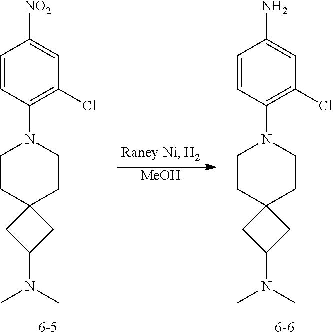 [SMILES: "MeOH" ; CN(C)C1CC2(CCN(c3ccc([N+](=O)[O-])cc3Cl)CC2)C1 ; CN(C)C1CC2(CCN(c3ccc(N)cc3Cl)CC2)C1]

To a solution of 7-(2-chloro-4-nitrophenyl)-N,N-dimethyl-7-azaspiro[3.5]nonan-2-amine (120 mg) dissolved in MeOH (5 mL) was added Raney Ni (60 mg). A balloon of H₂ gas was connected via a needle to the reaction mixture which was stirred at room temperature for 3 h. The solution was filtered through diatomite to remove the Raney Ni. The solution was evaporated to give 90 mg of 7-(4-amino-2-chlorophenyl)-N,N-dimethyl-7-azaspiro[3.5]nonan-2-amine. MS: 294 [M+H]⁺

Step 5: Synthesis of (2-((5-chloro-2-((3-chloro-4-(2-(dimethylamino)-7-azaspiro[3.5]nonan-7-yl)phenyl)amino)pyrimidin-4-yl)amino)phenyl)dimethylphosphine oxide

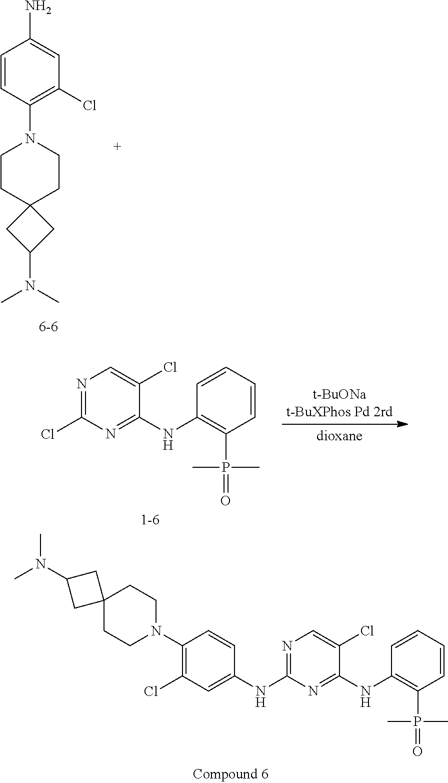

To a solution of 7-(4-amino-2-chlorophenyl)-N,N-dimethyl-7-azaspiro[3.5]nonan-2-amine (45 mg) and (2-((2,5-dichloropyrimidin-4-yl)amino)phenyl)dimethylphosphine oxide (73 mg) dissolved in dioxane (3 mL) was added t-BuONa (30 mg) and t-BuXPhos Pd 2rd (12 mg). The solution was purged with N₂ and stirred at 110° C. overnight. The reaction mixture was cooled down to room temperature and the solids were filtered out. The resulting mixture was concentrated. The residue was purified by column chromatography over silica gel with DCM/MeOH (8:1). This obtained 25 mg of compound 6 (2-((5-chloro-2-((3-chloro-4-(2-(dimethylamino)-7-azaspiro[3.5]nonan-7-yl)phenyl)amino)pyrimidin-4-yl)amino)phenyl)dimethylphosphine oxide. MS: 573 [M+H]⁺.

¹H NMR (500 MHz, Methanol-d₄) δ 8.41 (s, 1H), 8.08 (s, 1H), 7.71 (d, J=2.6 Hz, 1H), 7.60 (dt, J=16.5, 8.0 Hz, 2H), 7.27 (q, J=8.1 Hz, 2H), 6.99 (d, J=10.7 Hz, 1H), 2.90 (s, 2H), 2.83 (s, 2H), 2.76-2.71 (m, 1H), 2.16 (d, J=2.3 Hz, 6H), 2.10 (d, J=9.1 Hz, 2H), 1.84 (d, J=15.5 Hz, 6H), 1.78 (t, J=5.5 Hz, 2H), 1.71 (t, J=5.4 Hz, 2H), 1.66 (t, J=19.8 Hz, 2H).

### Example 7

### Synthesis of Compound 7

### (2-((2-((3-chloro-4-(2-(dimethylamino)-7-azaspiro[3.5]nonan-7-yl)phenyl)amino)pyrimidin-4-yl)amino)phenyl)dimethylphosphine oxide

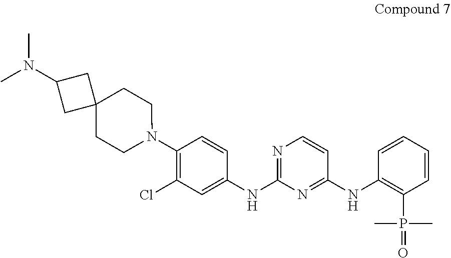

Step 1: Synthesis of (2((2-chloropyrimidin-4-yl)amino)phenyl)dimethylphosphine oxide

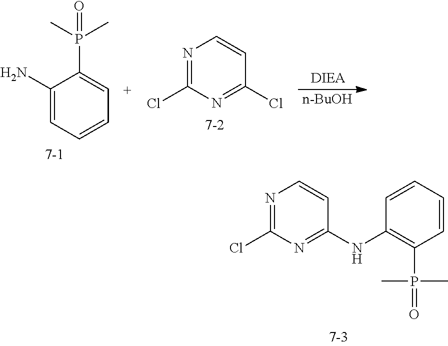

To a solution of (2-aminophenyl)dimethylphosphine oxide (2 g) and 2,4-dichloropyrimidine (1.76 g) dissolved in n-BuOH (20 mL) was added DIEA (2.1 g). The reaction mixture was stirred at 80° C. for 3 days. The reaction mixture was cooled down to room temperature and diluted with EtOAc (150 mL). The resulting solution was washed with water and NaCl saturated aqueous solution. The mixture was dried over anhydrous sodium sulfate and concentrated under vacuum. The residue was purified by column chromatography over silica gel with DCM/MeOH (15:1). This obtained 2.3 g of (2-((2-chloropyrimidin-4-yl)amino)phenyl)dimethylphosphine oxide as a yellow solid. MS: 282 [M+H]⁺

Step 2: Synthesis of (2-((2-((3-chloro-4-(2-(dimethylamino)-7-azaspiro[3.5]nonan-7-yl)phenyl)amino)pyrimidin-4-yl)amino)phenyl)dimethylphosphine oxide

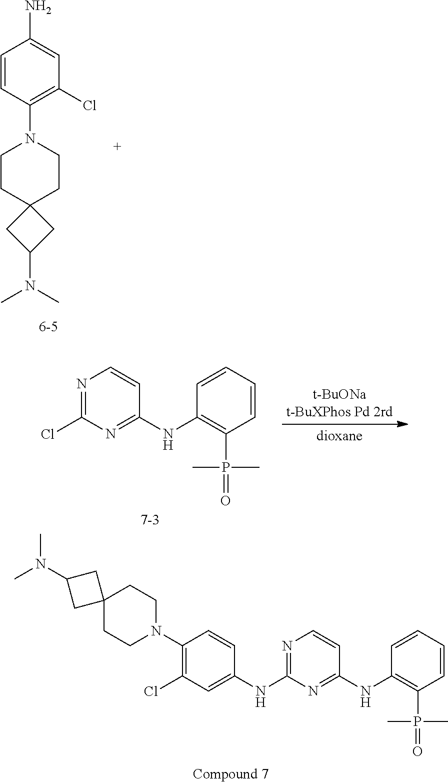

To a solution of 7-(4-amino-2-chlorophenyl)-N,N-dimethyl-7-azaspiro[3.5]nonan-2-amine (45 mg) and (2-((2-chloropyrimidin-4-yl)amino)phenyl)dimethylphosphine oxide (65 mg) dissolved in dioxane (3 mL) was added t-BuONa (30 mg) and t-BuXPhos Pd 2rd (12 mg). The solution was purged with N₂ and stirred at 110° C. overnight. The reaction mixture was cooled down to room temperature and the solids were filtered out. The resulting mixture was concentrated. The residue was purified by column chromatography over silica gel with DCM/MeOH (8:1). This obtained 21 mg of (2-((2-((3-chloro-4-(2-(dimethylamino)-7-azaspiro[3.5]nonan-7-yl)phenyl)amino)pyrimidin-4-yl)amino)phenyl)dimethylphosphine oxide. MS: 539 [M+H]⁺

### Example 8

### Synthesis of Compound 8

### (2-((2-((4-(2-amino-7-azaspiro[3.5]nonan-7-yl)-3-chlorophenyl)amino)-5-chloropyrimidin-4-yl)amino)phenyl)dimethylphosphine oxide

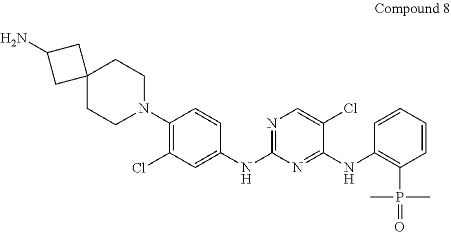 [SMILES: CP(C)(=O)c1ccccc1Nc1nc(Nc2ccc(N3CCC4(CC3)CC(N)C4)c(Cl)c2)ncc1Cl]

Step 1: Synthesis of tert-butyl (7-(4-amino-2-chlorophenyl)-7-azaspiro[3.5]nonan-2-yl)carbamate

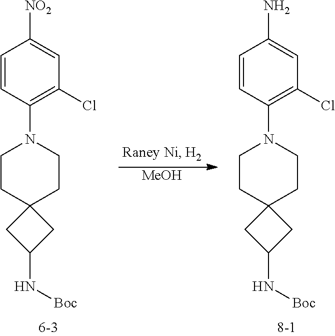 [SMILES: "MeOH" ; CC(C)(C)OC(=O)NC1CC2(CCN(c3ccc([N+](=O)[O-])cc3Cl)CC2)C1 ; CC(C)(C)OC(=O)NC1CC2(CCN(c3ccc(N)cc3Cl)CC2)C1]

To a solution of tert-butyl (7-(2-chloro-4-nitrophenyl)-7-azaspiro[3.5]nonan-2-yl)carbamate (550 mg) dissolved in MeOH (10 mL) was added Raney Ni (300 mg). A balloon of H₂ gas was connected via a needle to the reaction mixture which was stirred at room temperature for 5 h. The solution was filtered through diatomite to remove the Raney Ni. The solution was evaporated to give 480 mg of tert-butyl (7-(4-amino-2-chlorophenyl)-7-azaspiro[3.5]nonan-2-yl)carbamate. MS: 366 [M+H]⁺

Step 2: Synthesis of tert-butyl(7-(2-chloro-4-((5-chloro-4-((2-(dimethylphosphoryl)phenyl)amino)pyrimidin-2-yl)amino)phenyl)-7-azaspiro[3.5]nonan-2-yl)carbamate

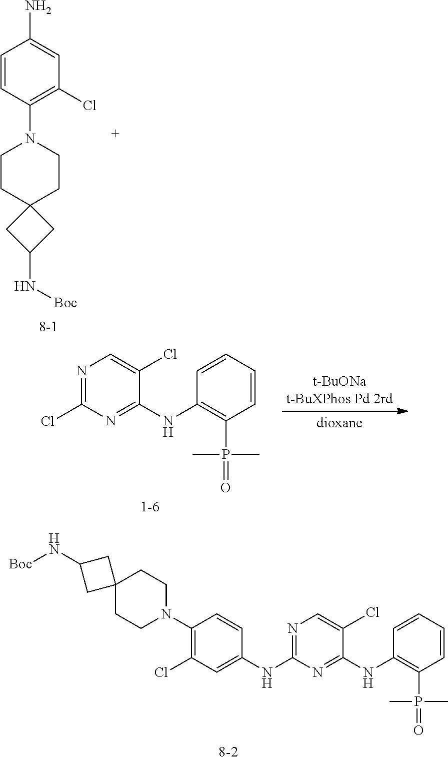 [SMILES: CC(C)(C)OC(=O)NC1CC2(CCN(c3ccc(N)cc3Cl)CC2)C1 ; CP(C)(=O)c1ccccc1Nc1nc(Cl)ncc1Cl ; CC(C)(C)OC(=O)NC1CC2(CCN(c3ccc(Nc4ncc(Cl)c(Nc5ccccc5P(C)(C)=O)n4)cc3Cl)CC2)C1]

To a solution of tert-butyl (7-(4-amino-2-chlorophenyl)-7-azaspiro[3.5]nonan-2-yl)carbamate (60 mg) and (2-((2,5-dichloropyrimidin-4-yl)amino)phenyl)dimethylphosphine oxide (80 mg) dissolved in dioxane (4 mL) was added t-BuONa (32 mg) and t-BuXPhos Pd 2rd (13 mg). The solution was purged with N₂ and stirred at 110° C. overnight. The reaction mixture was cooled down to room temperature and the solids were filtered out. The resulting mixture was concentrated. The residue was purified by column chromatography over silica gel with DCM/MeOH (15:1). This obtained 70 mg of tert-butyl(7-(2-chloro-4-((5-chloro-4-((2-(dimethylphosphoryl)phenyl)amino)pyrimidin-2-yl)amino)phenyl)-7-azaspiro[3.5]nonan-2-yl)carbamate as a yellow solid. MS: 645 [M+H]⁺

Step 3: Synthesis of (2-((2-((4-(2-amino-7-azaspiro[3.5]nonan-7-yl)-3-chlorophenyl)amino)-5-chloropyrimidin-4-yl)amino)phenyl)dimethylphosphine oxide hydrochloric acid salt

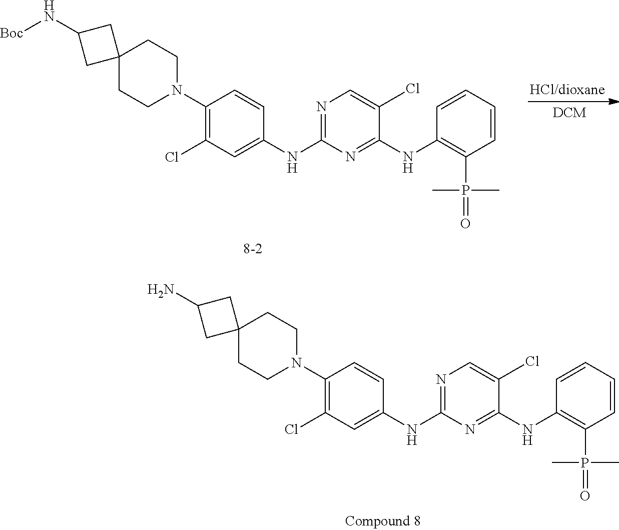 [SMILES: CC(C)(C)OC(=O)NC1CC2(CCN(c3ccc(Nc4ncc(Cl)c(Nc5ccccc5P(C)(C)=O)n4)cc3Cl)CC2)C1 ; CP(C)(=O)c1ccccc1Nc1nc(Nc2ccc(N3CCC4(CC3)CC(N)C4)c(Cl)c2)ncc1Cl]

To a stirred solution of tert-butyl (7-(2-chloro-4-((5-chloro-4-((2-(dimethylphosphoryl)phenyl)amino)pyrimidin-2-yl)amino)phenyl)-7-azaspiro[3.5]nonan-2-yl)carbamate (70 mg) in DCM (6 mL) was added HCl/dioxane (2 mL, 5N). The reaction mixture was stirred at room temperature for 0.5 h. The reaction mixture was evaporated under reduced pressure to give 62 mg of compound 8 (2-((2-((4-(2-amino-7-azaspiro[3.5]nonan-7-yl)-3-chlorophenyl)amino)-5-chloropyrimidin-4-yl)amino)phenyl)dimethylphosphine oxide hydrochloric acid salt. MS of Compound 8: 545 [M+H]⁺.

### Example 9

### Synthesis of Compound 9

### (2-((2-((4-(2-amino-7-azaspiro[3.5]nonan-7-yl)-3-chlorophenyl)amino)pyrimidin-4-yl)amino)phenyl)dimethylphosphine oxide

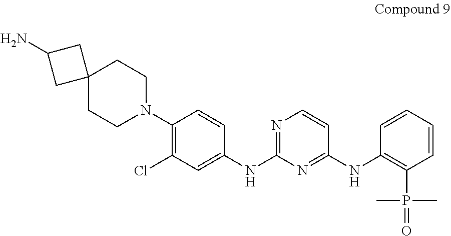 [SMILES: CP(C)(=O)c1ccccc1Nc1ccnc(Nc2ccc(N3CCC4(CC3)CC(N)C4)c(Cl)c2)n1]

Step 1: Synthesis of tert-butyl (7-(2-chloro-4-((4-((2-(dimethylphosphoryl)phenyl)amino)pyrimidin-2-yl)amino)phenyl)-7-azaspiro[3.5]nonan-2-yl)carbamate

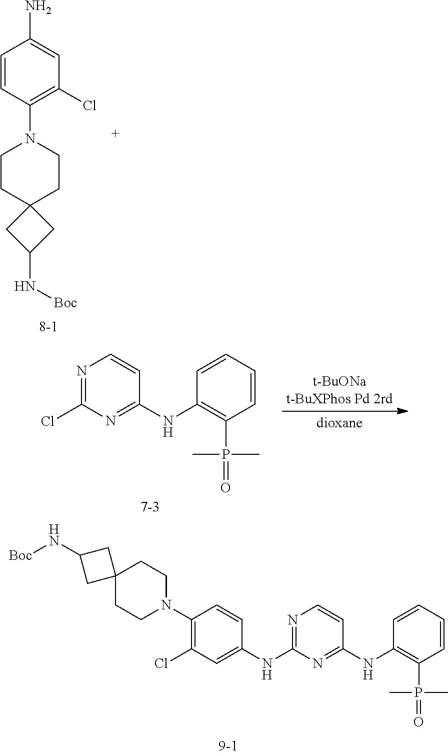 [SMILES: CC(C)(C)OC(=O)NC1CC2(CCN(c3ccc(N)cc3Cl)CC2)C1 ; CP(C)(=O)c1ccccc1Nc1ccnc(Cl)n1 ; CC(C)(C)OC(=O)NC1CC2(CCN(c3ccc(Nc4nccc(Nc5ccccc5P(C)(C)=O)n4)cc3Cl)CC2)C1]

To a solution of tert-butyl (7-(4-amino-2-chlorophenyl)-7-azaspiro[3.5]nonan-2-yl)carbamate (80 mg) and (2-((2-chloropyrimidin-4-yl)amino)phenyl)dimethylphosphine oxide (93 mg) dissolved in dioxane (4 mL) was added t-BuONa (42 mg) and t-BuXPhos Pd 2rd (18 mg). The solution was purged with N₂ and stirred at 110° C. overnight. The reaction mixture was cooled down to room temperature and the solids were filtered out. The resulting mixture was concentrated. The residue was purified by column chromatography over silica gel with DCM/MeOH (15:1). This obtained 50 mg of tert-butyl (7-(2-chloro-4-((4-((2-(dimethylphosphoryl)phenyl)amino)pyrimidin-2-yl)amino)phenyl)-7-azaspiro[3.5]nonan-2-yl)carbamate. MS: 611 [M+H]⁺

Step 2: Synthesis of (2-((2-((4-(2-amino-7-azaspiro[3.5]nonan-7-yl)-3-chlorophenyl)amino)pyrimidin-4-yl)amino)phenyl)dimethylphosphine oxide hydrochloric acid salt

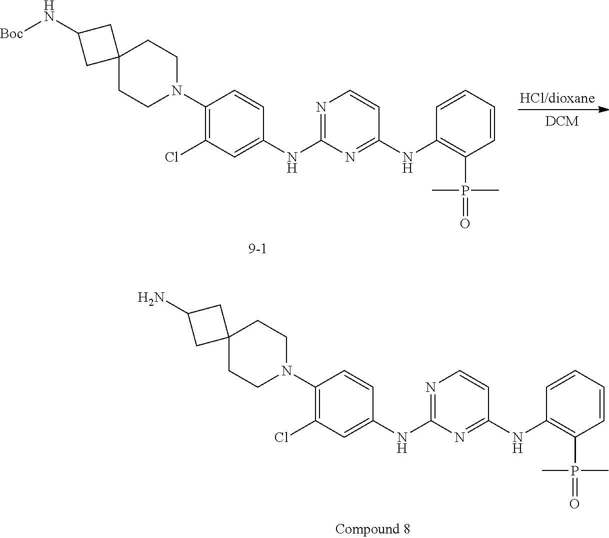 [SMILES: CC(C)(C)OC(=O)NC1CC2(CCN(c3ccc(Nc4nccc(Nc5ccccc5P(C)(C)=O)n4)cc3Cl)CC2)C1 ; CP(C)(=O)c1ccccc1Nc1ccnc(Nc2ccc(N3CCC4(CC3)CC(N)C4)c(Cl)c2)n1]

To a stirred solution of tert-butyl (7-(2-chloro-4-((4-((2-(dimethylphosphoryl)phenyl)amino)pyrimidin-2-yl)amino)phenyl)-7-azaspiro[3.5]nonan-2-yl)carbamate (50 mg) in DCM (5 mL) was added HCl/dioxane (0.5 mL, 5N). The reaction mixture was stirred at room temperature for 0.5 h. The reaction mixture was evaporated under reduced pressure to give 47 mg of (2-((2-((4-(2-amino-7-azaspiro[3.5]nonan-7-yl)-3-chlorophenyl)amino)pyrimidin-4-yl)amino)phenyl)dimethylphosphine oxide hydrochloric acid salt. MS of Compound 9: 511 [M+H]⁺

### Example 10

### Synthesis of Compound 10

### (2-((5-chloro-2-((3-chloro-4-(7-(dimethylamino)-2-azaspiro[3.5]nonan-2-yl)phenyl)amino)pyrimidin-4-yl)amino)phenyl)dimethylphosphine oxide

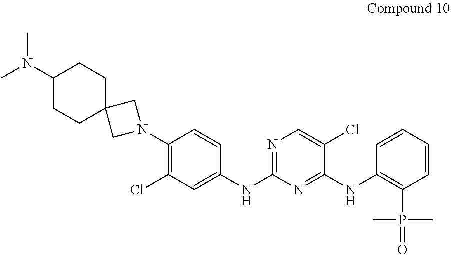

Step 1: Synthesis of 2-(2-chloro-4-nitrophenyl)-2-azaspiro[3.5]nonan-7-one

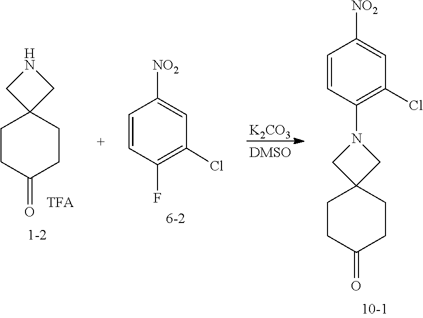

To a solution of 2-azaspiro[3.5]nonan-7-one trifluoroacetate (1.4 g) and 2-chloro-1-fluoro-4-nitrobenzene (0.73 g) dissolved in DMSO (20 mL) was added K₂CO₃ (3.5 g). The reaction mixture was stirred at 90° C. overnight. The reaction mixture was cooled down to room temperature and diluted with EtOAc (100 mL). The resulting solution was washed with water and NaCl saturated aqueous solution. The mixture was dried over anhydrous sodium sulfate and concentrated under vacuum. The residue was purified by column chromatography over silica gel with ethyl acetate/petroleum ether (1:3). This obtained 510 mg of 2-(2-chloro-4-nitrophenyl)-2-azaspiro[3.5]nonan-7-one as a yellow solid. MS: 295 [M+H]⁺

Step 2: Synthesis of 2-(4-amino-2-chlorophenyl)-2-azaspiro[3.5]nonan-7-one

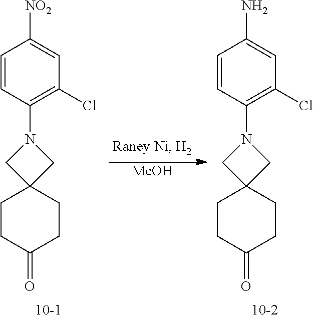 [SMILES: "MeOH" ; O=C1CCC2(CC1)CN(c1ccc([N+](=O)[O-])cc1Cl)C2 ; Nc1ccc(N2CC3(CCC(=O)CC3)C2)c(Cl)c1]

To a solution of 2-(2-chloro-4-nitrophenyl)-2-azaspiro[3.5]nonan-7-one (510 mg) dissolved in MeOH (10 mL) was added Raney Ni (500 mg). A balloon of H₂ gas was connected via a needle to the reaction mixture which was stirred at room temperature for 3 h. The solution was filtered through diatomite to remove the Raney Ni. The solution was evaporated to give 310 mg of 2-(4-amino-2-chlorophenyl)-2-azaspiro[3.5]nonan-7-one. MS: 265 [M+H]⁺

Step 3: Synthesis of 2-(2-chloro-4-((5-chloro-4-((2-(dimethylphosphoryl)phenyl)amino)pyrimidin-2-yl)amino)phenyl)-2-azaspiro[3.5]nonan-7-one

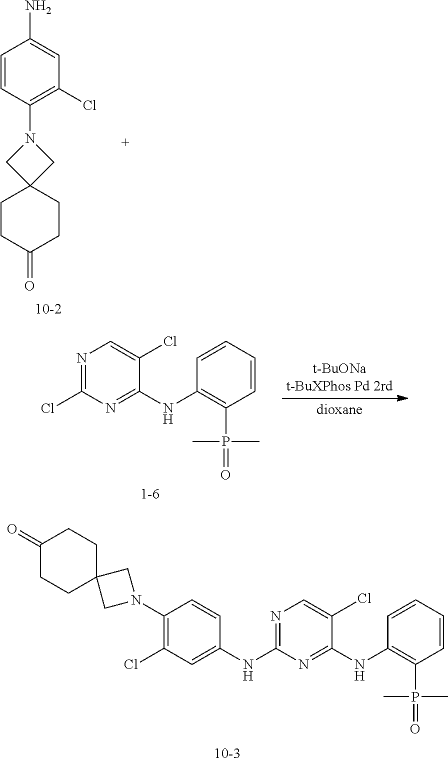 [SMILES: Nc1ccc(N2CC3(CCC(=O)CC3)C2)c(Cl)c1 ; CP(C)(=O)c1ccccc1Nc1nc(Cl)ncc1Cl ; CP(C)(=O)c1ccccc1Nc1nc(Nc2ccc(N3CC4(CCC(=O)CC4)C3)c(Cl)c2)ncc1Cl]

To a solution of 2-(4-amino-2-chlorophenyl)-2-azaspiro[3.5]nonan-7-one (290 mg) and (2-((2,5-dichloropyrimidin-4-yl)amino)phenyl)dimethylphosphine oxide (341 mg) dissolved in dioxane (6 mL) was added t-BuONa (138 mg) and t-BuXPhos Pd 2rd (60 mg). The solution was purged with N₂ and stirred at 120° C. for 4 h by Microwave. The reaction mixture was cooled down to room temperature and the solids were filtered out. The resulting mixture was concentrated. The residue was purified by column chromatography over silica gel with DCM/MeOH (15:1). This obtained 110 mg of 2-(2-chloro-4-((5-chloro-4-((2-(dimethylphosphoryl)phenyl)amino)pyrimidin-2-yl)amino)phenyl)-2-azaspiro[3.5]nonan-7-one. MS: 544 [M+H]⁺

Step 4: Synthesis of (2-((5-chloro-2-((3-chloro-4-(7-(dimethylamino)-2-azaspiro[3.5]nonan-2-yl)phenyl)amino)pyrimidin-4-yl)amino)phenyl)dimethylphosphine oxide

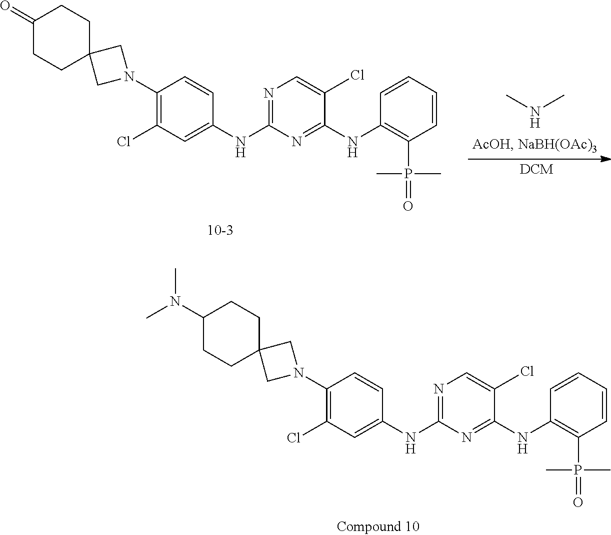

To a solution of 2-(2-chloro-4-((5-chloro-4-((2-(dimethylphosphoryl)phenyl)amino)pyrimidin-2-yl)amino)phenyl)-2-azaspiro[3.5]nonan-7-one (80 mg) in DCM (6 mL) was added dimethylamine (0.5 mL, 2N in THF) and AcOH (3 drops). The mixture was stirred at room temperature. After 1 h, sodium triacetoxyborohydride (62 mg) was added and the mixture was further stirred at room temperature for 1 h. After completion of the reaction (monitored by TLC), the reaction mixture was diluted with DCM (20 mL). The resulting solution was washed with 10% NaHCO3 aqueous solution and NaCl saturated aqueous solution. The mixture was dried over anhydrous magnesium sulfate and concentrated under vacuum. The residue was purified by column chromatography over silica gel with DCM/MeOH (8:1). This obtained 40 mg of (2-((5-chloro-2-((3-chloro-4-(7-(dimethylamino)-2-azaspiro[3.5]nonan-2-yl)phenyl)amino)pyrimidin-4-yl)amino)phenyl)dimethylphosphine oxide.

MS of Compound 10: 573 [M+H]⁺. ¹H NMR (500 MHz, Methanol-d₄) δ 8.42 (s, 1H), 8.05 (s, 1H), 7.63-7.54 (m, 3H), 7.28-7.19 (m, 2H), 6.56 (d, J=8.5 Hz, 1H), 3.78 (s, 2H), 3.68 (s, 2H), 2.96 (d, J=12.4 Hz, 1H), 2.72 (s, 6H), 2.15 (d, J=13.2Hz, 2H), 2.04-1.97 (m, 2H), 1.85 (d, J=15.3 Hz, 6H), 1.63 (t, J=13.2Hz, 2H), 1.52 (q, J=12.4 Hz, 2H).

### Example 11

### Synthesis of Compound 11

### (2-((2-((4-(7-amino-2-azaspiro[3.5]nonan-2-yl)-3-chlorophenyl)amino)-5-chloropyrimidin-4-yl)amino)phenyl)dimethylphosphine oxide

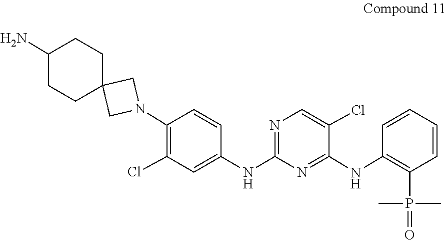 [SMILES: CP(C)(=O)c1ccccc1Nc1nc(Nc2ccc(N3CC4(CCC(N)CC4)C3)c(Cl)c2)ncc1Cl]

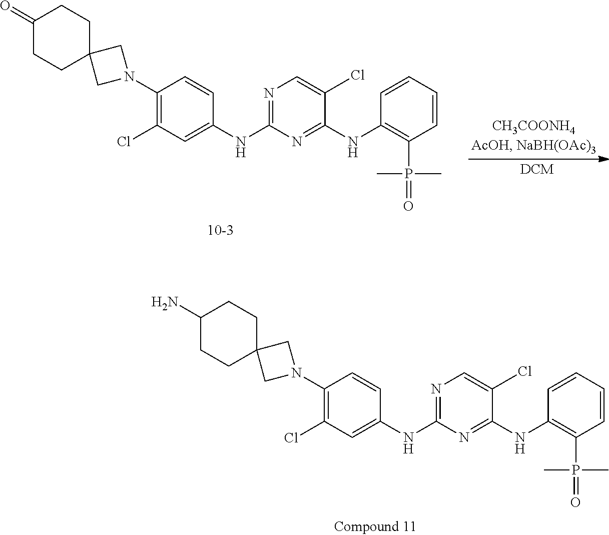 [SMILES: CP(C)(=O)c1ccccc1Nc1nc(Nc2ccc(N3CC4(CCC(=O)CC4)C3)c(Cl)c2)ncc1Cl ; CP(C)(=O)c1ccccc1Nc1nc(Nc2ccc(N3CC4(CCC(N)CC4)C3)c(Cl)c2)ncc1Cl]

To a solution of 2-(2-chloro-4-((5-chloro-4-((2-(dimethylphosphoryl)phenyl)amino)pyrimidin-2-yl)amino)phenyl)-2-azaspiro[3.5]nonan-7-one (50 mg) in DCM (4 mL) was added ammonium acetate (35 mg) and AcOH (1 drop). The mixture was stirred at room temperature. After 1 h, sodium triacetoxyborohydride (38 mg) was added and the mixture was further stirred at room temperature for 1 h. After completion of the reaction (monitored by TLC), the reaction mixture was diluted with DCM (10 mL). The resulting solution was washed with 10% NaHCO3 aqueous solution and NaCl saturated aqueous solution. The mixture was dried over anhydrous magnesium sulfate and concentrated under vacuum. The residue was purified by column chromatography over silica gel with DCM/MeOH (8:1). This obtained 4.5 mg of (2-((2-((4-(7-amino-2-azaspiro[3.5]nonan-2-yl)-3-chlorophenyl)amino)-5-chloropyrimidin-4-yl)amino)phenyl)dimethylphosphine oxide. MS of Compound 11: 545 [M+H]⁺

### Example 12

### Synthesis of Compound 12

### (2-((5-chloro-2-((3-chloro-4-(7-((2-hydroxyethyl)amino)-2-azaspiro[3.5]nonan-2-yl)phenyl)amino)pyrimidin-4-yl)amino)phenyl)dimethylphosphine oxide

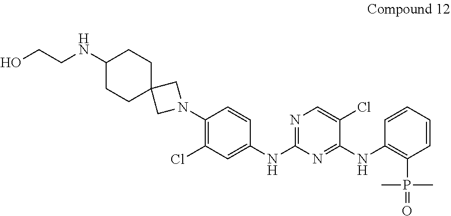 [SMILES: CP(C)(=O)c1ccccc1Nc1nc(Nc2ccc(N3CC4(CCC(NCCO)CC4)C3)c(Cl)c2)ncc1Cl]

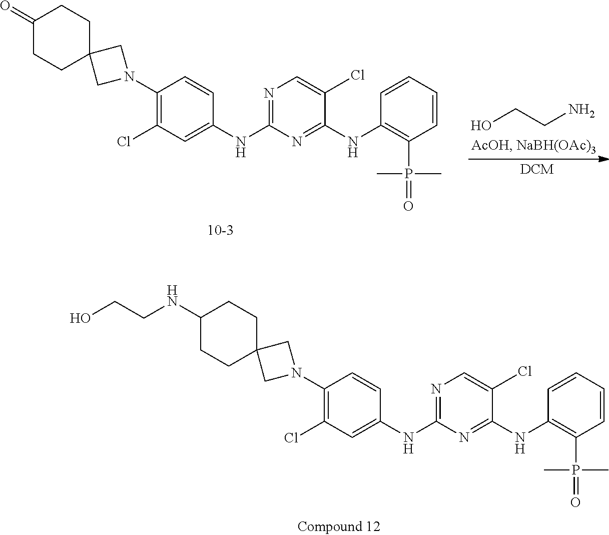

To a solution of 2-(2-chloro-4-((5-chloro-4-((2-(dimethylphosphoryl)phenyl)amino)pyrimidin-2-yl)amino)phenyl)-2-azaspiro[3.5]nonan-7-one (50 mg) in DCM (4 mL) was added 2-aminoethan-1-ol (17 mg) and AcOH (1 drop). The mixture was stirred at room temperature. After 1 h, sodium triacetoxyborohydride (39 mg) was added and the mixture was further stirred at room temperature for 1 h. After completion of the reaction (monitored by TLC), the reaction mixture was diluted with DCM (10 mL). The resulting solution was washed with 10% NaHCO3 aqueous solution and NaCl saturated aqueous solution. The mixture was dried over anhydrous magnesium sulfate and concentrated under vacuum. The residue was purified by column chromatography over silica gel with DCM/MeOH (8:1). This obtained 33 mg of (2-((5-chloro-2-((3-chloro-4-(7-((2-hydroxyethyl)amino)-2-azaspiro[3.5]nonan-2-yl)phenyl)amino)pyrimidin-4-yl)amino)phenyl)dimethylphosphine oxide. MS of Compound 12: 589 [M+H]⁺.

### Example 13

### Synthesis of Compound 13

### (2-((5-chloro-2-((4-(7-(dimethylamino)-2-azaspiro[3.5]nonan-2-yl)phenyl)amino)pyrimidin-4-yl)amino)phenyl)dimethylphosphine oxide

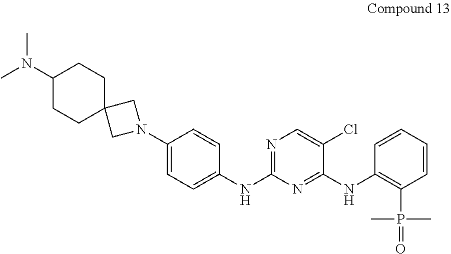

Step 1: Synthesis of 2-(4-nitrophenyl)-2-azaspiro[3.5]nonan-7-one

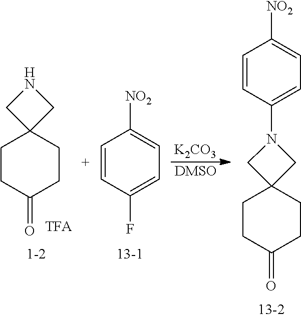

To a solution of 2-azaspiro[3.5]nonan-7-one trifluoroacetate (0.8 g) and 1-fluoro-4-nitrobenzene (0.30 g) dissolved in DMSO (10 mL) was added K₂CO₃ (1.74 g). The reaction mixture was stirred at 90° C. overnight. The reaction mixture was cooled down to room temperature and diluted with EtOAc (50 mL). The resulting solution was washed with water and NaCl saturated aqueous solution. The mixture was dried over anhydrous sodium sulfate and concentrated under vacuum. The residue was purified by column chromatography over silica gel with ethyl acetate/petroleum ether (1:3). This obtained 240 mg of 2-(4-nitrophenyl)-2-azaspiro[3.5]nonan-7-one. MS: 261 [M+14]⁺

Step 2: Synthesis of 2-(4-aminophenyl)-2-azaspiro[3.5]nonan-7-one

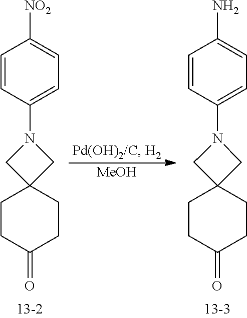

To a solution of 2-(4-nitrophenyl)-2-azaspiro[3.5]nonan-7-one (280 mg) dissolved in MeOH (10 mL) was added Palladium Hydroxide 20% on Carbon (100 mg, 50% water). A balloon of H₂ gas was connected via a needle to the reaction mixture which was stirred at room temperature for 3 h. The solution was filtered through diatomite to remove the Pd(OH)2/C. The solution was evaporated to give 170 mg of 2-(4-aminophenyl)-2-azaspiro[3.5]nonan-7-on. MS: 231 [M+H]⁺

Step 3: Synthesis of 2-(4-((5-chloro-4-((2-(dimethylphosphoryl)phenyl)amino)pyrimidin-2-yl)amino)phenyl)-2-azaspiro[3.5]nonan-7-one

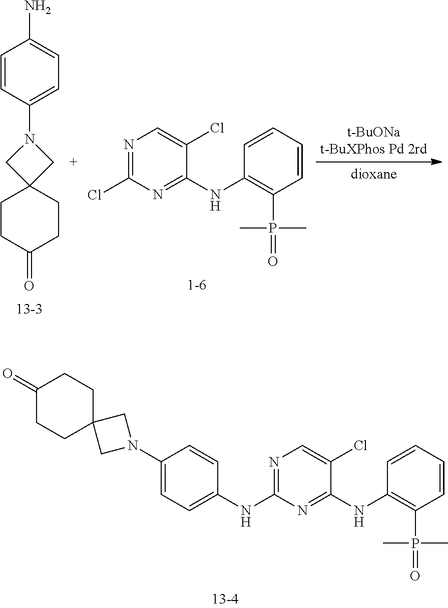

To a solution of 2-(4-aminophenyl)-2-azaspiro[3.5]nonan-7-one (170 mg) and (2-((2,5-dichloropyrimidin-4-yl)amino)phenyl)dimethylphosphine oxide (350 mg) dissolved in dioxane (8 mL) was added t-BuONa (142 mg) and t-BuXPhos Pd 2rd (60 mg). The solution was purged with N₂ and stirred at 120° C. for 4 h by Microwave. The reaction mixture was cooled down to room temperature and the solids were filtered out. The resulting mixture was concentrated. The residue was purified by column chromatography over silica gel with DCM/MeOH (15:1). This obtained 25 mg of 2-(4-((5-chloro-4-((2-(dimethylphosphoryl)phenyl)amino)pyrimidin-2-yl)amino)phenyl)-2-azaspiro[3.5]nonan-7-one. MS: 510 [M+H]⁺

Step 4: Synthesis of (2-((5-chloro-2-((4-(7-(dimethylamino)-2-azaspiro[3.5]nonan-2-yl)phenyl)amino)pyrimidin-4-yl)amino)phenyl)dimethylphosphine oxide

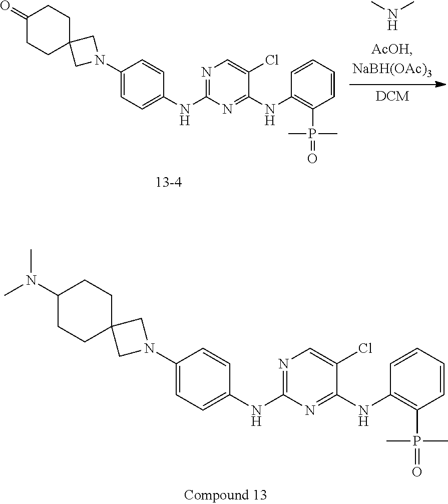

To a solution of 2-(4-((5-chloro-4-((2-(dimethylphosphoryl)phenyl)amino)pyrimidin-2-yl)amino)phenyl)-2-azaspiro[3.5]nonan-7-one (25 mg) in DCM (2 mL) was added dimethylamine (0.2 mL, 2N in THF) and AcOH (1 drop). The mixture was stirred at room temperature. After 1 h, sodium triacetoxyborohydride (21 mg) was added and the mixture was further stirred at room temperature overnight. The reaction mixture was diluted with DCM (10 mL) and washed with 10% NaHCO3 aqueous solution and NaCl saturated aqueous solution. The mixture was dried over anhydrous magnesium sulfate and concentrated under vacuum. The residue was purified by column chromatography over silica gel with DCM/MeOH (8:1). This obtained 10 mg of (2-((5-chloro-2-((4-(7-(dimethyl amino)-2-azaspiro [3.5]nonan-2-yl)phenyl)amino)pyrimidin-4-yl)amino)phenyl)dimethylphosphine oxide. MS: 539 [M+H]⁺

¹H NMR (500 MHz, Methanol-d₄) δ 8.46 (s, 1H), 7.99 (s, 1H), 7.57 (dd, J=14.3, 7.8 Hz, 1H), 7.48 (t, J=8.0 Hz, 1H), 7.29 (d, J=8.2Hz, 2H), 7.21 (t, J=7.6 Hz, 1H), 6.43 (d, J=8.3 Hz, 2H), 3.62 (s, 2H), 3.53 (s, 2H), 3.12 (t, J=11.9 Hz, 1H), 2.80 (s, 6H), 2.15 (d, J=13.1 Hz, 2H), 2.04 (d, 2H), 1.84 (d, J=13.4 Hz, 6H), 1.66 (t, J=13.4 Hz, 2H), 1.58 (t, J=12.2Hz, 2H).

### Example 14

### Synthesis of Compound 14

### (2-((2-((4-(2-amino-7-azaspiro[3.5]nonan-7-yl)-3-methylphenyl)amino)-5-chloropyrimidin-4-yl)amino)phenyl)dimethylphosphine oxide

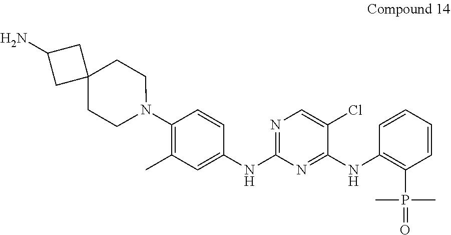 [SMILES: Cc1cc(Nc2ncc(Cl)c(Nc3ccccc3P(C)(C)=O)n2)ccc1N1CCC2(CC1)CC(N)C2]

Step 1:Synthesis of (2-((2,5-dichloropyrimidin-4-yl)amino)phenyl)dimethylphosphine oxide

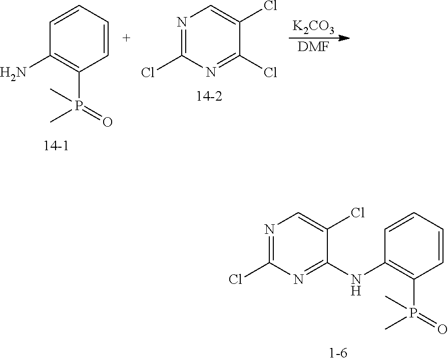

To a mixture of (2-aminophenyl)dimethylphosphine oxide (2.50 g) in DMF (30 mL), 2,4,5-trichloropyrimidine (3.52 g) and potassium carbonate (4.08 g) was added under stirring. The mixture was heated 60° C. for about 8 h. The mixture solution was poured into water and extracted with ethyl acetate (50 mL*3). The combined organic layer was washed with saturated sodium chloride aqueous solution (50 mL*2), dried over Na₂SO₄ and concentrated under reduced pressure. The crude product was recrystallized by hexane/ethyl acetate (10 : 1, 10 mL). After filtration, the solid was dried to obtain (2-((2,5-dichloropyrimidin-4-yl)amino)phenyl)dimethylphosphine oxide (3.00 g) as white solid. MS: 316 [M+H]⁺.

Step 2: Synthesis of tert-butyl (7-(2-methyl-4-nitrophenyl)-7-azaspiro[3.5]nonan-2-yl)carbamate

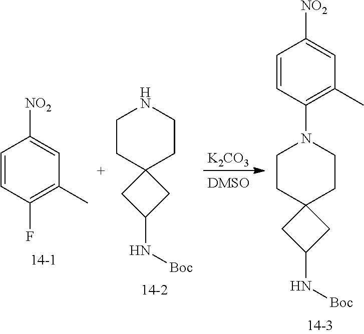

To a mixture of 2-fluoro-5-nitrotoluene (250 mg) in DMSO (10 mL), tert-butyl 7-azaspiro[3.5]nonan-2-ylcarbamate (465 mg) and potassium carbonate (446 mg) was added under stirring. The mixture was heated 90° C. for about 10 h. The mixture solution was poured into water and extracted with ethyl acetate (20 mL*3). The combined organic layer was washed with saturated sodium chloride aqueous solution (20 mL*2), dried over Na₂SO₄ and concentrated to give crude product, which was purified by silica gel column chromatography using hexane/ethyl acetate (4:1) as the eluent, and to obtain tert-butyl (7-(2-methyl-4-nitrophenyl)-7-azaspiro[3.5]nonan-2-yl)carbamate (500 mg) as yellow solid. MS: 376 [M+H]⁺.

Step 3: Synthesis of tert-butyl(7-(4-amino-2-methylphenyl)-7-azaspiro[3.5]nonan-2-yl)carbamate

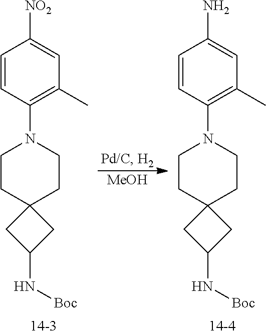

To a solution of tert-butyl (7-(2-methyl-4-nitrophenyl)-7-azaspiro[3.5]nonan-2-yl) carbamate (500 mg) in methanol (20 mL) was added 10% palladium on carbon (100 mg) and the mixture hydrogenated (hydrogen balloon) at room temperature for 5 h. The mixture was then filtered through diatomaceous earth and washed with methanol; the filtrate was then concentrated under reduced pressure to afford the tert-butyl(7-(4-amino-2-methylphenyl) -7-azaspiro[3.5]nonan-2-yl)carbamate (300 mg) as white solid. MS: 346 [M+H]⁺.

Step 4: Synthesis of tert-butyl(7-(4-((5-chloro-4-((2-(dimethylphosphoryl)phenyl)amino)pyrimidin-2-yl)amino)-2-methylphenyl)-7-azaspiro[3.5]nonan-2-yl)carbamate

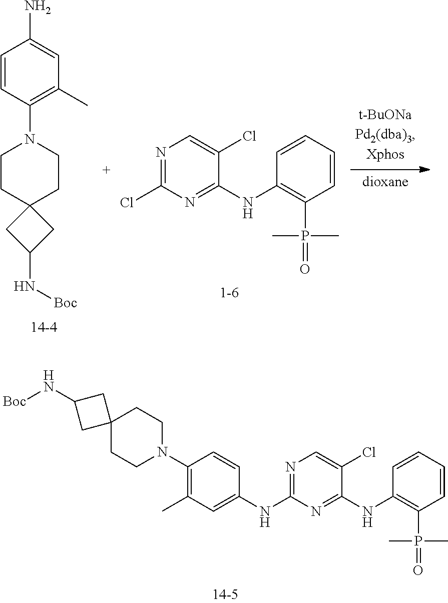 [SMILES: Cc1cc(N)ccc1N1CCC2(CC1)CC(NC(=O)OC(C)(C)C)C2 ; CP(C)(=O)c1ccccc1Nc1nc(Cl)ncc1Cl ; Cc1cc(Nc2ncc(Cl)c(Nc3ccccc3P(C)(C)=O)n2)ccc1N1CCC2(CC1)CC(NC(=O)OC(C)(C)C)C2]

To a solution of (2-((2,5-dichloropyrimidin-4-yl)amino)phenyl)dimethylphosphine oxide (200 mg) in dioxane (5 mL) was added tert-butyl(7-(4-amino-2-methylphenyl)-7-azaspiro[3.5]nonan-2-yl)carbamate (200 mg), Xphos (51 mg), Pd₂(dba)₃ (49 mg) and t-BuONa (153 mg). The mixture was charged with nitrogen, and heated 100° C. for about 12 h. The reaction mixture was concentrated and purified by silica gel column chromatography using DCM/methanol (95: 5) as the eluent, and to obtain tert-butyl (7-(4-((5-chloro-4-((2-(dimethylphosphoryl)phenyl) amino) pyrimidin-2-yl)amino)-2-methylphenyl) -7-azaspiro[3.5]nonan-2-y0 carbamate (100 mg) as brown solid. MS: 625 [M+H]⁺.

Step 5: Synthesis of (2-((2-((4-(2-amino-7-azaspiro[3.5]nonan-7-yl)-3-methylphenyl)amino)-5-chloropyrimidin-4-yl)amino)phenyl)dimethylphosphine oxide hydrochloric acid salt

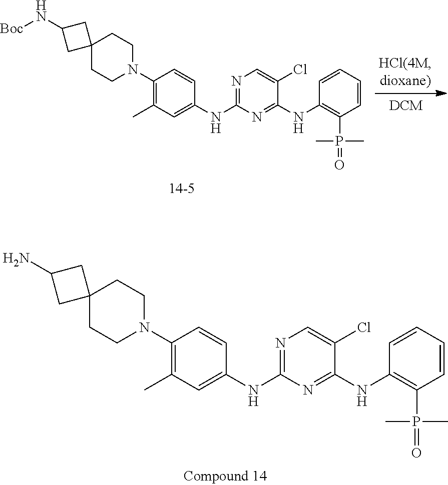 [SMILES: Cc1cc(Nc2ncc(Cl)c(Nc3ccccc3P(C)(C)=O)n2)ccc1N1CCC2(CC1)CC(NC(=O)OC(C)(C)C)C2 ; Cc1cc(Nc2ncc(Cl)c(Nc3ccccc3P(C)(C)=O)n2)ccc1N1CCC2(CC1)CC(N)C2]

To a solution of tert-butyl (7-(4-((5-chloro-4-((2-(dimethylphosphoryl) phenyl) amino) pyrimidin-2-yl)amino)-2-methylphenyl)-7-azaspiro[3.5]nonan-2-yl)carbamate (200 mg) in DCM was added HCl (4M, dioxane, 0.5 mL). The mixture was stirred 5 h at room temperature. After filtration, the solid was washed with DCM and dried to obtain (2-((2-((4-(2-amino-7-azaspiro[3.5]nonan-7-yl)-3-methylphenyl)amino)-5-chloropyrimidin-4-yl)amino)phenyl) dimethylphosphine oxide hydrochloric acid salt (30 mg) as white solid. MS of Compound 14: 525 [M+H]⁺.

¹H NMR (500 MHz, DMSO-d6) δ 11.37-11.22 (m, 2H) 9.89 (s, 1H) 8.51 (s, 1H) 8.30 (d, J=15.50 Hz, 3H) 7.72-7.48 (m, 3H) 7.24-7.23 (m, 1H) 4.89 (s, 2H) 3.73-3.72 (m, 1H) 3.44 (s, 3H) 2.51-2.50 (m, 8H) 2.14-2.09 (m, 4H) 1.81-1.78 (m, 6H).

### Example 15

### Synthesis of Compound 15

### (2-((5-chloro-2-((4-(2-(dimethylamino)-7-azaspiro[3.5]nonan-7-yl)-3-methylphenyl)amino)pyrimidin-4-yl)amino)phenyl)dimethylphosphine oxide

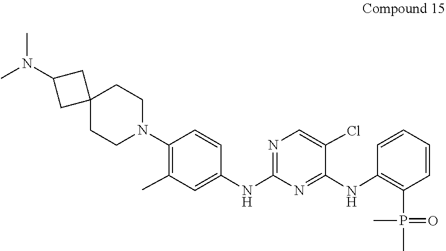 [SMILES: Cc1cc(Nc2ncc(Cl)c(Nc3ccccc3P(C)(C)=O)n2)ccc1N1CCC2(CC1)CC(N(C)C)C2]

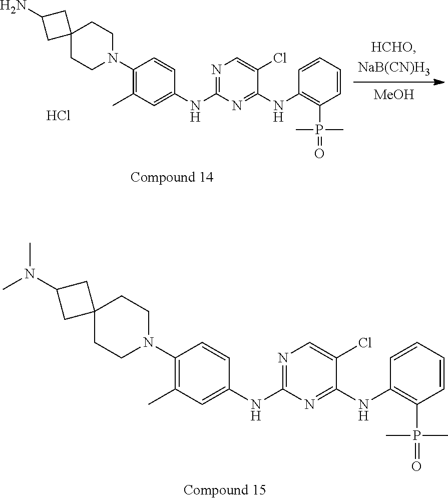

To a solution of compound 14 (150 mg) in methanol (10 mL) was added HCHO (50 mg). The mixture was stirred 30 min at room temperature. Then the reaction mixture was added NaB(CN)H₃ (110 mg), and stirred another 2 h. The reaction mixture was concentrated, purified by silica gel column chromatography using DCM/methanol (95:5) as the eluent. The product was dissolved in DCM, added HCl (0.1 mL), was then concentrated under reduced pressure to afford (2-((5-chloro-2-((4-(2-(dimethylamino)-7-azaspiro[3.5]nonan-7-yl)-3-methylphenyl)amino)pyrimidin-4-yl)amino)phenyl)dimethylphosphine oxide hydrochloric acid salt (120 mg) as pale yellow solid. MS: 553 [M+H]+.

### Example 16

### Synthesis of Compound 16

### (2-((2-((4-(2-amino-7-azaspiro[3.5]nonan-7-yl)phenyl)amino)-5-chloropyrimidin-4-yl)amino)phenyl)dimethylphosphine oxide hydrochloride

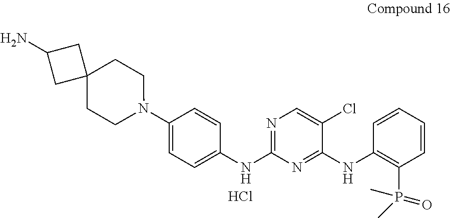 [SMILES: CP(C)(=O)c1ccccc1Nc1nc(Nc2ccc(N3CCC4(CC3)CC(N)C4)cc2)ncc1Cl ; Cl]

Step 1: Synthesis of tert-butyl (7-(4-nitrophenyl)-7-azaspiro[3.5]nonan-2-yl)carbamate

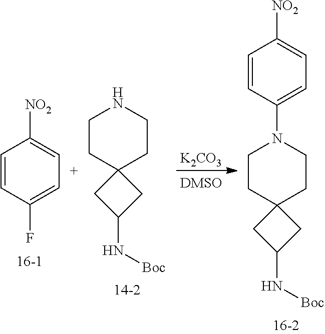

Following the same procedure as tert-butyl (7-(2-methyl-4-nitrophenyl)-7-azaspiro[3.5]nonan-2-yl)carbamate using 4-fluoronitrobenzene instead of 2-fluoro-5-nitrotoluene with tert-butyl 7-azaspiro[3.5]nonan-2-ylcarbamate and potassium carbonate to obtain tert-butyl (7-(4-nitrophenyl)-7-azaspiro[3.5]nonan-2-yl)carbamate. MS: 361.44 [M+H]⁺.

Step 2: Synthesis of tert-butyl (7-(4-aminophenyl)-7-azaspiro[3.5]nonan-2-yl)carbamate

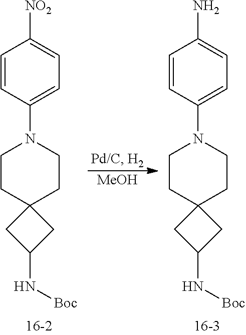

Following the same procedure as tert-butyl(7-(4-amino-2-methylphenyl)-7-azaspiro[3.5]nonan-2-yl)carbamate using tert-butyl (7-(4-nitrophenyl)-7-azaspiro[3.5]nonan-2-yl)carbamate instead of tert-butyl (7-(2-methyl-4-nitrophenyl)-7-azaspiro[3.5]nonan-2-yl)carbamate to obtain tert-butyl (7-(4-aminophenyl)-7-azaspiro[3.5]nonan-2-yl)carbamate. MS: 331 [M+H]+.

Step 3: Synthesis of tert-butyl(7-(4-((5-chloro-4-((2-(dimethylphosphoryl)phenyl)amino)pyrimidin-2-yl)amino)phenyl)-7-azaspiro[3.5]nonan-2-yl)carbamate

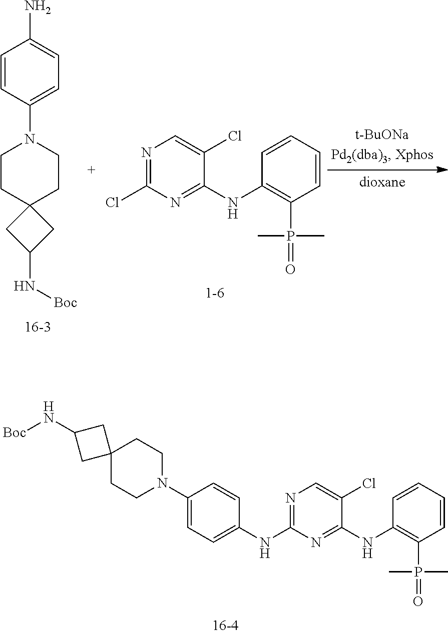 [SMILES: CC(C)(C)OC(=O)NC1CC2(CCN(c3ccc(N)cc3)CC2)C1 ; CP(C)(=O)c1ccccc1Nc1nc(Cl)ncc1Cl ; CC(C)(C)OC(=O)NC1CC2(CCN(c3ccc(Nc4ncc(Cl)c(Nc5ccccc5P(C)(C)=O)n4)cc3)CC2)C1]

Following the same procedure as tert-butyl(7-(4-((5-chloro-4-((2-(dimethylphosphoryl)phenyl)amino)pyrimidin-2-yl)amino)-2-methylphenyl)-7-azaspiro[3.5]nonan-2-yl)carbamate using tert-butyl (7-(4-aminophenyl)-7-azaspiro[3.5]nonan-2-yl)carbamate instead of tert-butyl(7-(4-amino-2-methylphenyl)-7-azaspiro[3.5]nonan-2-yl)carbamate to obtain tert-butyl(7-(4-((5-chloro-4-((2-(dimethylphosphoryl)phenyl)amino)pyrimidin-2-yl)amino)phenyl)-7-azaspiro[3.5]nonan-2-yl)carbamate. MS: 625 [M+H]+.

Step 4: Synthesis of (2-((2-((4-(2-amino-7-azaspiro[3.5]nonan-7-yl)phenyl)amino)-5-chloropyrimidin-4-yl)amino)phenyl)dimethylphosphine oxide hydrochloric acid salt

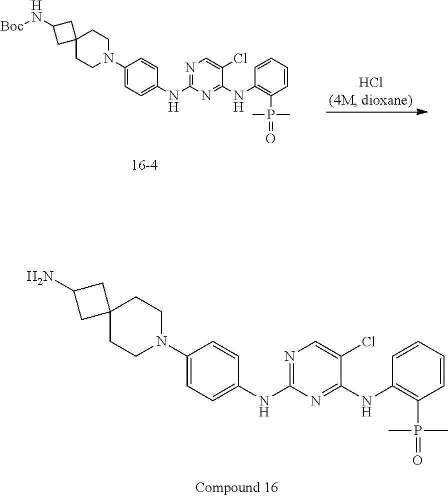 [SMILES: CC(C)(C)OC(=O)NC1CC2(CCN(c3ccc(Nc4ncc(Cl)c(Nc5ccccc5P(C)(C)=O)n4)cc3)CC2)C1 ; CP(C)(=O)c1ccccc1Nc1nc(Nc2ccc(N3CCC4(CC3)CC(N)C4)cc2)ncc1Cl]

Following the same procedure as (2-((2-((4-(2-amino-7-azaspiro[3.5]nonan-7-yl)-3-methylphenyl)amino)-5-chloropyrimidin-4-yl)amino)phenyl)dimethylphosphine oxide hydrochloric acid salt using tert-butyl (7-(4-((5-chloro-4-((2-(dimethylphosphoryl)phenyl)amino)pyrimidin-2-yl)amino)phenyl)-7-azaspiro[3.5]nonan-2-yl)carbamate instead of tert-butyl (7-((4-((5-chloro-4-((2-(dimethylphosphoryl)phenyl)amino)pyrimidin-2-yl)amino)-2-methylphenyl)-7-azaspiro[3.5]nonan-2-yl)carbamate to obtain (2-((2-((4-(2-amino-7-azaspiro[3.5]nonan-7-yl)phenyl)amino)-5-chloropyrimidin-4-yl)amino)phenyl)dimethylphosphine oxide hydrochloric acid salt. MS of Compound 16: 525 [M+H]+.

### Example 17

### Synthesis of Compound 17

### (2-((5-chloro-2-((4-(2-(dimethylamino)-7-azaspiro[3.5]nonan-7-yl)phenyl)amino)pyrimidin-4-yl)amino)phenyl)dimethylphosphine oxide

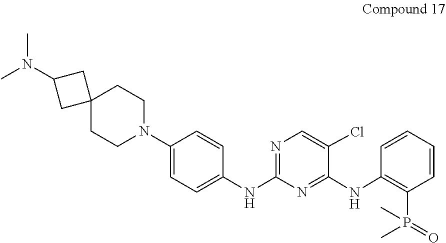 [SMILES: CN(C)C1CC2(CCN(c3ccc(Nc4ncc(Cl)c(Nc5ccccc5P(C)(C)=O)n4)cc3)CC2)C1]

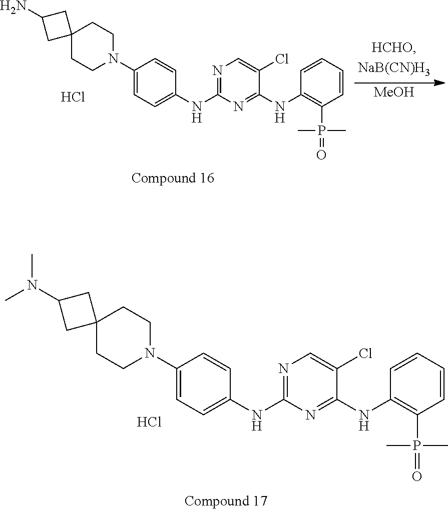

Following the same procedure as compound 15, using compound 16 instead of compound 14 to obtain compound 17 (2-((5-chloro-2-((4-(2-(dimethylamino)-7-azaspiro[3.5]nonan-7-yl)phenyl)amino)pyrimidin-4-yl)amino)phenyl)dimethylphosphine oxide hydrochloric acid salt. MS: 539 [M+H]+.

¹H NMR (500 MHz, DMSO-d6) δ 12.84 (s, 1H) 11.40 (s, 2H) 9.97 (s, 1H) 8.52 (s, 1H) 8.29 (s, 1H) 7.78 (s, 4H),7.68-7.56 (m, 2H) 7.26-7.23 (m, 1H) 3.71-3.70 (m, 1H) 3.50-3.40 (m, 4H) 2.63-2.62 (m, 6H) 2.30-1.90 (m, 8H) 1.81-1.78 (m, 6H).

### Example 18

### Synthesis of Compound 18

### (2-((2-((4-(2-amino-7-azaspiro[3.5]nonan-7-yl)-3-(trifluoromethyl)phenyl)amino)-5-chloropyrimidin-4-yl)amino)phenyl)dimethylphosphine oxide

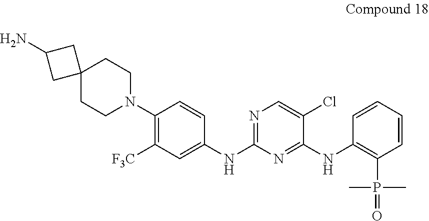 [SMILES: CP(C)(=O)c1ccccc1Nc1nc(Nc2ccc(N3CCC4(CC3)CC(N)C4)c(C(F)(F)F)c2)ncc1Cl]

Step 1: Synthesis of tert-butyl(7-(4-nitro-2-(trifluoromethyl)phenyl)-7-azaspiro[3.5]nonan-2-yl)carbamate

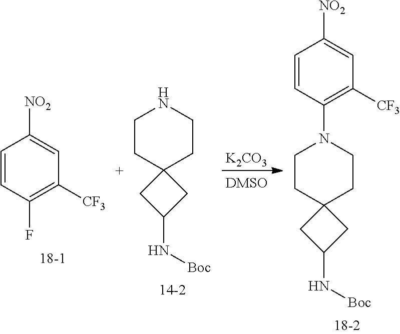

Following the same procedure as tert-butyl (7-(2-methyl-4-nitrophenyl)-7-azaspiro[3.5]nonan-2-yl)carbamate using 2-fluoro-5-nitrobenzotrifluoride instead of 2-fluoro-5-nitrotoluene with tert-butyl 7-azaspiro[3.5]nonan-2-ylcarbamate and potassium carbonate to obtain tert-butyl (7-(4-nitro-2-(trifluoromethyl)phenyl)-7-azaspiro[3.5]nonan-2-yl)carbamate. MS: 429 [M+H]⁺.

Step 2: Synthesis of tert-butyl(7-(4-amino-2-(trifluoromethyl)phenyl)-7-azaspiro[3.5]nonan-2-yl)carbamate

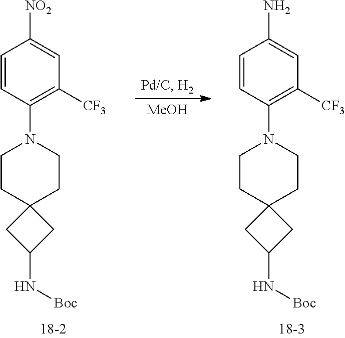 [SMILES: "MeOH" ; CC(C)(C)OC(=O)NC1CC2(CCN(c3ccc([N+](=O)[O-])cc3C(F)(F)F)CC2)C1 ; "Pd/C, H2" ; CC(C)(C)OC(=O)NC1CC2(CCN(c3ccc(N)cc3C(F)(F)F)CC2)C1]

Following the same procedure as tert-butyl(7-(4-amino-2-methylphenyl)-7-azaspiro[3.5]nonan-2-yl)carbamate using tert-butyl(7-(4-nitro-2-(trifluoromethyl)phenyl)-7-azaspiro[3.5]nonan-2-yl)carbamate instead of tert-butyl (7-(2-methyl-4-nitrophenyl)-7-azaspiro[3.5]nonan-2-yl)carbamate to obtain tert-butyl(7-(4-amino-2-(trifluoromethyl)phenyl)-7-azaspiro[3.5]nonan-2-yl) carbamate. MS: 399 [M+H]⁺.

Step 3: Synthesis of tert-butyl(7-(4-((5-chloro-4-((2-(dimethylphosphoryl)phenyl)amino)pyrimidin-2-yl)amino)-2-(trifluoromethyl)phenyl)-7-azaspiro[3.5]nonan-2-yl)carbamate

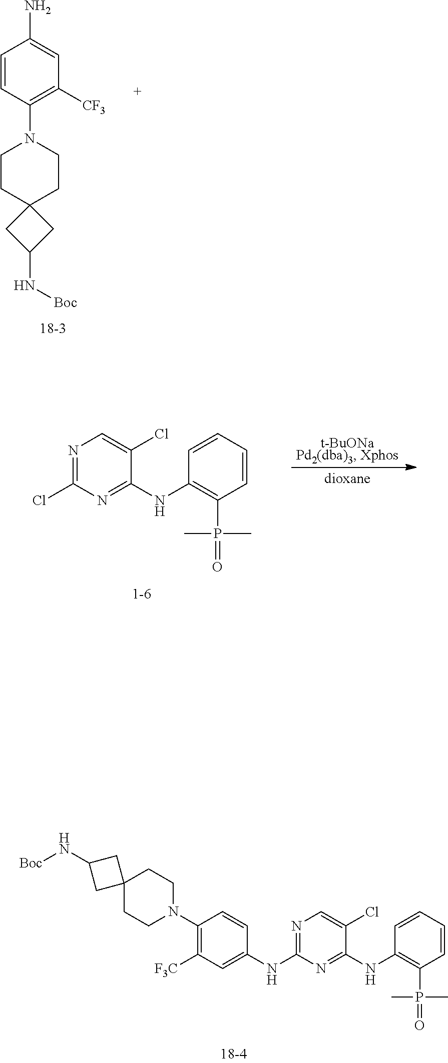 [SMILES: CC(C)(C)OC(=O)NC1CC2(CCN(c3ccc(N)cc3C(F)(F)F)CC2)C1 ; CP(C)(=O)c1ccccc1Nc1nc(Cl)ncc1Cl ; "dioxane" ; "t-BuONaPd2(dba)3, Xphos" ; CC(C)(C)OC(=O)NC1CC2(CCN(c3ccc(Nc4ncc(Cl)c(Nc5ccccc5P(C)(C)=O)n4)cc3C(F)(F)F)CC2)C1]

Following the same procedure as tert-butyl(7-(4-((5-chloro-4-((2-(dimethylphosphoryl)phenyl)amino)pyrimidin-2-yl)amino)-2-methylphenyl)-7-azaspiro[3.5]nonan-2-yl)carbamate using tert-butyl(7-(4-amino-2-(trifluoromethyl)phenyl)-7-azaspiro[3.5]nonan-2-yl)carbamate instead of tert-butyl(7-(4-amino-2-methylphenyl)-7-azaspiro[3.5]nonan-2-yl)carbamate obtain tert-butyl(7-(4-((5-chloro-4-((2-(dimethylphosphoryl)phenyl)amino)pyrimidin-2-yl)amino)-2-(trifluoromethyl)phenyl)-7-azaspiro[3.5]nonan-2-yl)carbamate. MS: 679 [M+H]⁺.

Step 4: Synthesis of (2-((2-((4-(2-amino-7-azaspiro[3.5]nonan-7-yl)-3-(trifluoromethyl)phenyl)amino)-5-chloropyrimidin-4-yl)amino)phenyl)dimethylphosphine oxide hydrochloric acid salt

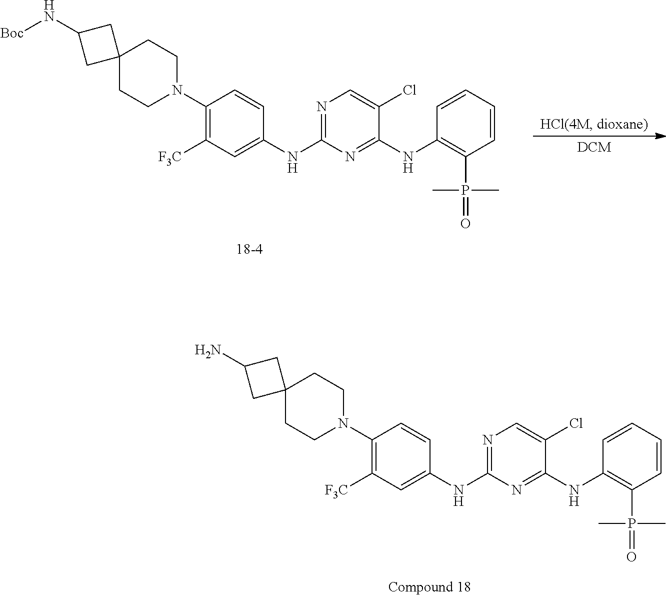

Following the same procedure as (2-((2-((4-(2-amino-7-azaspiro[3.5]nonan-7-yl)-3-methylphenyl)amino)-5-chloropyrimidin-4-yl)amino)phenyl)dimethylphosphine oxide hydrochloric acid salt using (7-(4-((5-chloro-4-((2-(dimethylphosphoryl)phenyl)amino)pyrimidin-2-yl)amino)-2-(trifluoromethyl)phenyl)-7-azaspiro[3.5]nonan-2-yl) carbamate instead of tert-butyl (7-((4-((5-chloro-4-((2-(dimethylphosphoryl)phenyl)amino)pyrimidin-2-yl)amino)-2-methylphenyl)-7-azaspiro[3.5]nonan-2-yl)carbamate to obtain (2-((2-((4-(2-amino-7-azaspiro[3.5]nonan-7-yl)-3-(trifluoromethyl)phenyl)amino)-5-chloropyrimidin-4-yl)amino)phenyl)dimethylphosphine oxide hydrochloric acid salt. MS of Compound 18: 579 [M+H]⁺.

### Example 19

### Synthesis of Compound 19

### (2-((5-chloro-2-((4-(2-(dimethylamino)-7-azaspiro[3.5]nonan-7-yl)-3-(trifluoromethyl)phenyl)amino)pyrimidin-4-yl)amino)phenyl)dimethylphosphine oxide

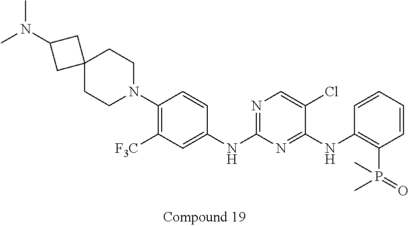 [SMILES: CN(C)C1CC2(CCN(c3ccc(Nc4ncc(Cl)c(Nc5ccccc5P(C)(C)=O)n4)cc3C(F)(F)F)CC2)C1]

Step 1: Synthesis of (2-((5-chloro-2-((4-(2-(dimethylamino)-7-azaspiro[3.5]nonan-7-yl)-3-(trifluoromethyl)phenyl)amino)pyrimidin-4-yl)amino)phenyl)dimethylphosphine oxide

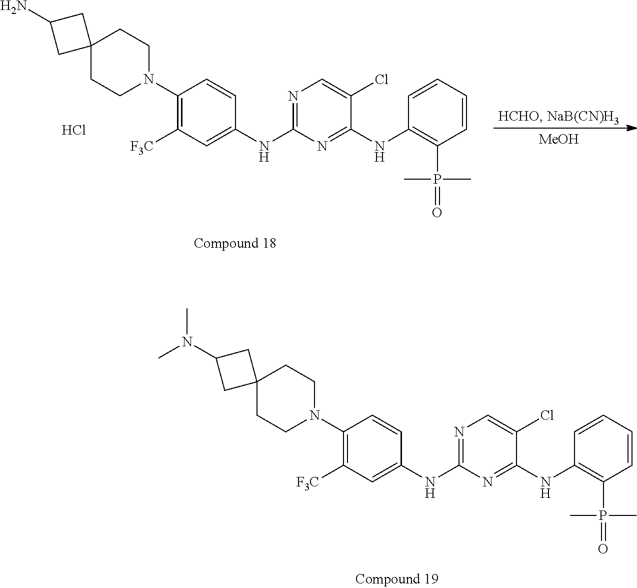

Following the same procedure as compound 15, using compound 18 instead of compound 14 to obtain (2-((5-chloro-2-((4-(2-(dimethylamino)-7-azaspiro[3.5]nonan-7-yl)-3-(trifluoromethyl)phenyl)amino)pyrimidin-4-yl)amino)phenyl)dimethylphosphine oxide. MS of Compound 19: 607 [M+H]⁺.

### Example 20

### Synthesis of Compound 20

### (2-((5-chloro-2-((3-chloro-4-(2-(methylamino)-7-azaspiro[3.5]nonan-7-yl)phenyl)amino)pyrimidin-4-yl)amino)phenyl)dimethylphosphine oxide

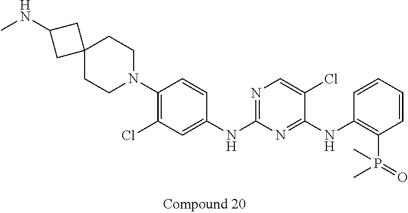 [SMILES: CNC1CC2(CCN(c3ccc(Nc4ncc(Cl)c(Nc5ccccc5P(C)(C)=O)n4)cc3Cl)CC2)C1]

Step 1: Synthesis of 7-(2-chloro-4-nitrophenyl)-7-azaspiro[3.5]nonan-2-one

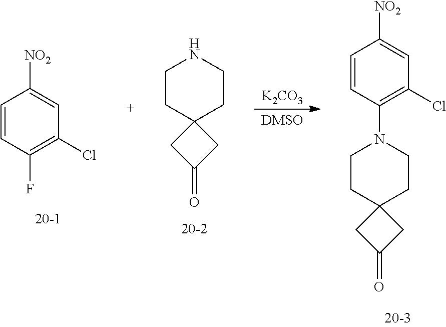

Following the same procedure as tert-butyl (7-(2-methyl-4-nitrophenyl)-7-azaspiro[3.5]nonan-2-yl)carbamate using 3-chloro-4-fluoronitrobenzene instead of 2-fluoro-5-nitroluene with 7-azaspiro[3.5]nonan-2-one hydrochloride and potassium carbonate to obtain 7-(2-chloro-4-nitrophenyl)-7-azaspiro[3.5]nonan-2-one. MS: 295 [M+H]⁺.

Step 2: Synthesis of 7-(4-amino-2-chlorophenyl)-7-azaspiro[3.5]nonan-2-one

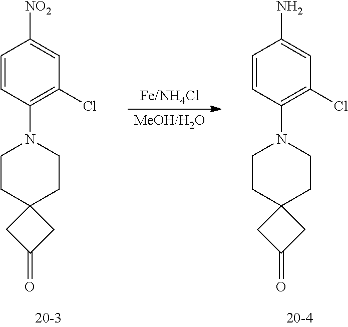 [SMILES: O=C1CC2(CCN(c3ccc([N+](=O)[O-])cc3Cl)CC2)C1 ; "Fe/NH4Cl" ; "MeOH/H2O" ; Nc1ccc(N2CCC3(CC2)CC(=O)C3)c(Cl)c1]

Following the same procedure as 2-(4-amino-2-chlorophenyl)hexahydrocyclopenta[c]pyrrol-5(1H)-one using 7-(2-chloro-4-nitrophenyl)-7-azaspiro[3.5]nonan-2-one instead of 2-(2-chloro-4-nitrophenyl)hexahydrocyclopenta[c]pyrrol-5(1H)-one to obtain 7-(4-amino-2-chlorophenyl)-7-azaspiro[3.5]nonan-2-one. MS: 265 [M+H]⁺.

Step 3: Synthesis of 7-(2-chloro-4-((5-chloro-4-((2-(dimethylphosphoryl)phenyl)amino)pyrimidin-2-yl)amino)phenyl)-7-azaspiro[3.5]nonan-2-one

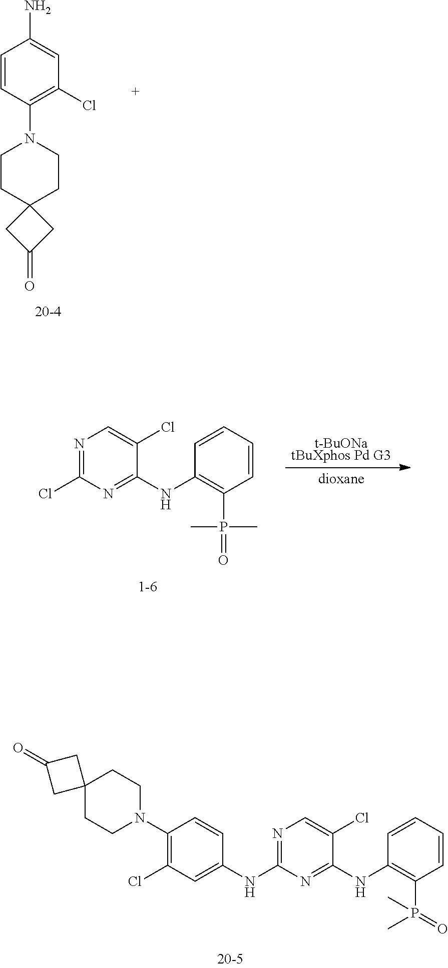 [SMILES: Nc1ccc(N2CCC3(CC2)CC(=O)C3)c(Cl)c1 ; CP(C)(=O)c1ccccc1Nc1nc(Cl)ncc1Cl ; "dioxane" ; "t-BuONatBuXphos Pd G3" ; CP(C)(=O)c1ccccc1Nc1nc(Nc2ccc(N3CCC4(CC3)CC(=O)C4)c(Cl)c2)ncc1Cl]

Following the same procedure as 2-(2-chloro-4-((5-chloro-4-((2-(dimethylphosphoryl)phenyl)amino)pyrimidin-2-yl)amino)phenyl)hexahydrocyclopenta[c] pyrrol-5(1H)-one using 7-(4-amino-2-chlorophenyl)-7-azaspiro[3.5]nonan-2-one instead of 2-(4-amino-2-chlorophenyl)hexahydrocyclopenta[c]pyrrol-5 (1H)-one to obtain 7-(2-chloro-4-((5-chloro-4-((2-(dimethylphosphoryl)phenyl)amino)pyrimidin-2-yl)amino)phenyl)-7-azaspiro[3.5]nonan-2-one. MS: 544 [M+H]⁺.

Step 4: Synthesis of (2-((5-chloro-2-((3-chloro-4-(2-(methylamino)-7-azaspiro[3.5]nonan-7-yl)phenyl)amino)pyrimidin-4-yl)amino)phenyl)dimethylphosphine oxide

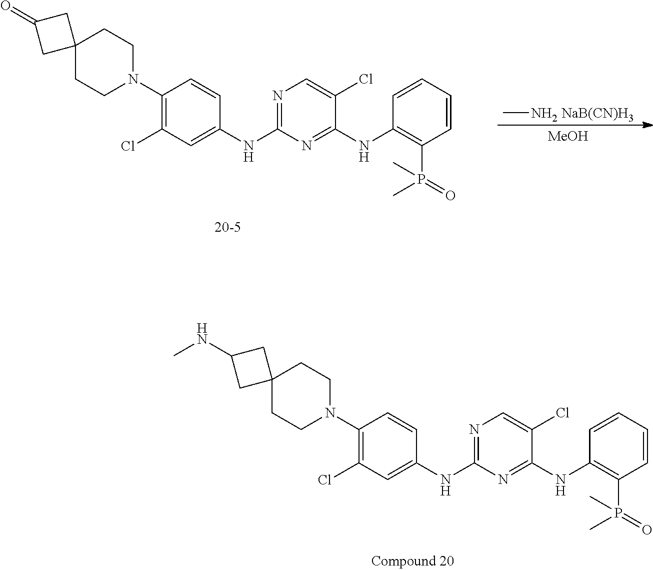

To a solution of 7-(2-chloro-4-((5-chloro-4-((2-(dimethylphosphoryl)phenyl)amino)pyrimidin-2-yl)amino)phenyl)-7-azaspiro[3.5]nonan-2-one (30 mg) in methanol (5 mL) was added methylamine THF solution (2M, 0.5 mL). The mixture was heated 50° C. 10 h. Then the reaction mixture was added NaB(CN)H₃ (100 mg), and stirred another 2 h. The reaction mixture was concentrated, purified by silica gel column chromatography using DCM/methanol (95:5) as the eluent to afford (2-((5-chloro-2-((3-chloro-4-(2-(methylamino)-7-azaspiro[3.5]nonan-7-yl)phenyl)amino)pyrimidin-4-yl)amino)phenyl)dimethylphosphine oxide.

MS of Compound 20: 559 [M+H]⁺.

### Example 21

### Synthesis of Compound 21

### (2-((2-((4-(2-amino-7-azaspiro[3.5]nonan-7-yl)-3-methylphenyl)amino)-9H-purin-6-yl)amino)phenyl)dimethylphosphine oxide

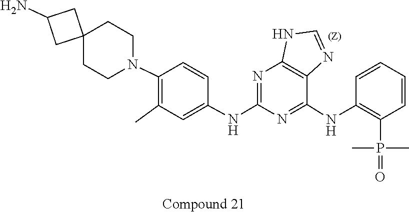 [SMILES: Cc1cc(Nc2nc(Nc3ccccc3P(C)(C)=O)c3nc[nH]c3n2)ccc1N1CCC2(CC1)CC(N)C2]

Step 1: Synthesis of 2,6-dichloro-9-(4-methoxybenzyl)-9H-purine

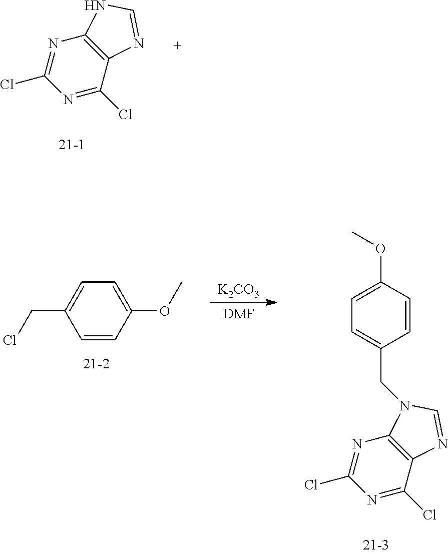

To a mixture of 2,6-dichloropurine (2.00 g) in DMF (20 mL), 4-methoxybenzylchloride (1.99 g) and potassium carbonate (2.92 g) was added under stirring. The mixture was stirring about 12 h at room temperature. The mixture solution was poured into water and extracted with ethyl acetate (50 mL*3). The combined organic layer was washed with saturated sodium chloride aqueous solution (20 mL*2), dried over Na₂SO₄ and and concentrated to give crude product, which purified by silica gel column chromatography using hexane/ethyl acetate (1:1) as the eluent, and to obtain 2,6-dichloro-9-(4-methoxybenzyl)-9H-purine (1.50 g). MS: 309 [M+H]⁺.

Step 2: Synthesis of (2-((2-chloro-9-(4-methoxybenzyl)-9H-purin-6-yl)amino)phenyl)dimethylphosphine oxide

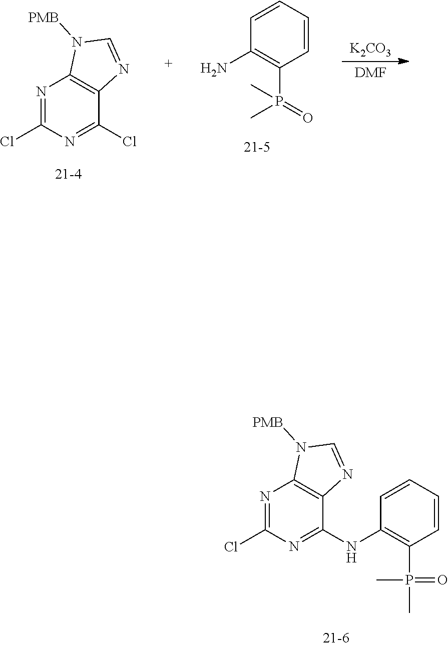

To a mixture of (2-aminophenyl)dimethylphosphine oxide (380 mg) in DMF (10 mL), 2,6-dichloro-9-(4-methoxybenzyl)-9H-purine (700 mg) and potassium carbonate (939 mg) was added under stirring. The mixture was heated 80° C. for about 12 h. The mixture solution was poured into water and extracted with ethyl acetate (50 mL*3). The combined organic layer was washed with saturated sodium chloride aqueous solution (20 mL*2), dried over Na₂SO₄ and and concentrated to give crude product, which purified by silica gel column chromatography using DCM/methanol (95:5) as the eluent, and to obtain (2-((2-chloro-9-(4-methoxybenzyl)-9H-purin-6-yl)amino)phenyl)dimethylphosphine oxide (300 mg). MS: 442 [M+H]⁺.

Step 3: Synthesis of tert-butyl(7-(4-((6-((2-(dimethylphosphoryl)phenyl)amino)-9-(4-methoxybenzyl)-9H-purin-2-yl)amino)-2-methylphenyl)-7-azaspiro[3.5]nonan-2-yl)carbamate

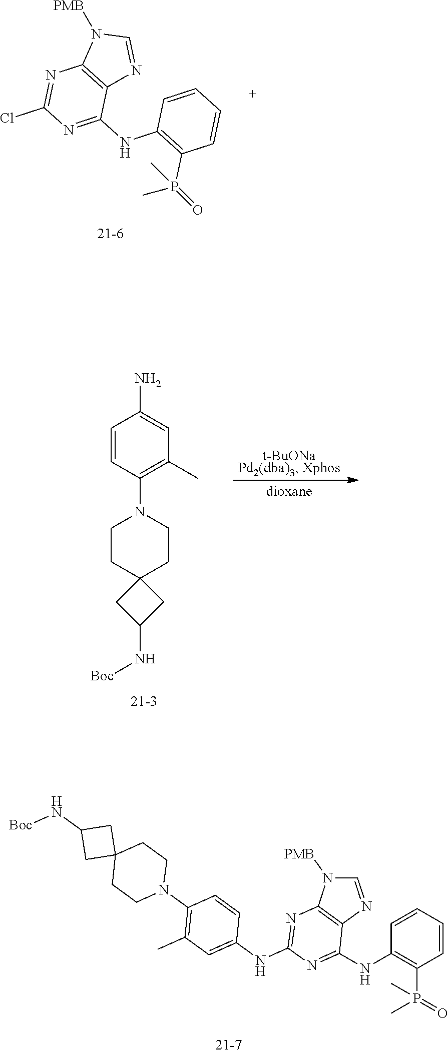

Following the same procedure as tert-butyl(7-(4-((5-chloro-4-((2-(dimethylphosphoryl)phenyl)amino)pyrimidin-2-yl)amino)-2-methylphenyl)-7-azaspiro[3.5]nonan-2-yl)carbamate using (2-42-chloro-9-(4-methoxybenzyl)-9H-purin-6-yl)amino)phenyl)dimethylphosphine oxide instead of tert-butyl(7-(4-amino-2-methylphenyl)-7-azaspiro[3.5]nonan-2-yl)carbamate obtain tert-butyl(7-(4-((6-((2-(dimethylphosphoryl)phenyl)amino)-9-(4-methoxybenzyl)-9H-purin-2-yl)amino)-2-methylphenyl)-7-azaspiro[3.5]nonan-2-yl)carbamate (100 mg). MS: 751 [M+H]⁺.

Step 4: Synthesis of (2-((2-((4-(2-amino-7-azaspiro[3.5]nonan-7-yl)-3-methylphenyl)amino)-9H-purin-6-yl)amino)phenyl)dimethylphosphine oxide trifluoroacetate

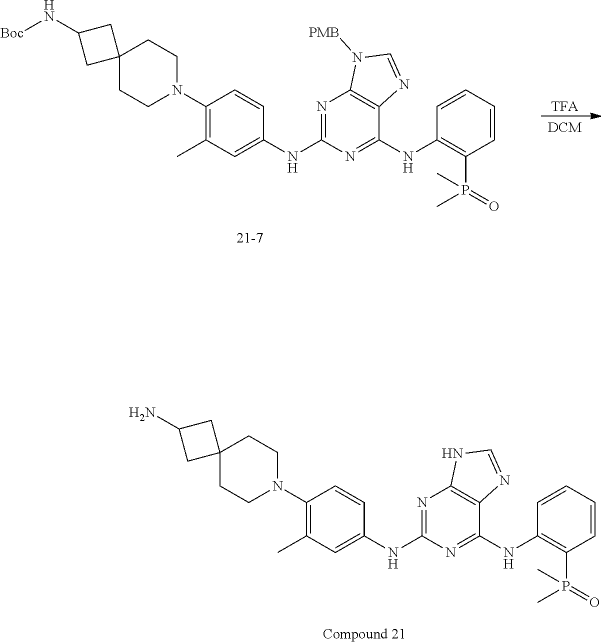

To a solution of tert-butyl (7-(4-((6-((2-(dimethylphosphoryl)phenyl)amino)-9-(4-methoxybenzyl)-9H-purin-2-yl)amino)-2-methylphenyl)-7-azaspiro[3.5]nonan-2-yl)carbamate (100 mg) in DCM was added TFA (5 mL). The mixture was concentrated under reduced pressure. The mixture was stirred 5 h at room temperature. The crude product was recrystallized by ethyl acetate (5 mL). After filtration, the solid was dried to obtain (2-((2-((4-(2-amino-7-azaspiro[3.5]nonan-7-yl)-3-methy 1phenyl)amino)-9H-purin-6-yl)amino)phenyl)dimethylphosphine oxide trifluoroacetate (63 mg) as white solid.

MS of Compound 21: 531 [M+H]⁺.

### Example 22

### Synthesis of Compound 22

### (2-((2-((4-(6-amino-2-azaspiro[3.3]heptan-2-yl)-3-methylphenyl)amino)-5-methylpyrimidin-4-yl)amino)phenyl)dimethylphosphine oxide

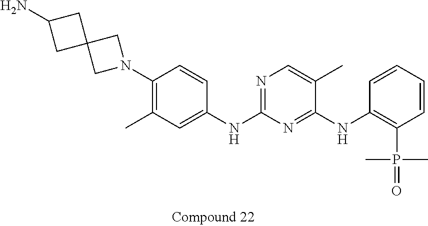 [SMILES: Cc1cc(Nc2ncc(C)c(Nc3ccccc3P(C)(C)=O)n2)ccc1N1CC2(CC(N)C2)C1]

Step 1: Synthesis of tert-butyl (2-(2-methyl-4-nitrophenyl)-2-azaspiro[3.3]heptan-6-yl)carbamate

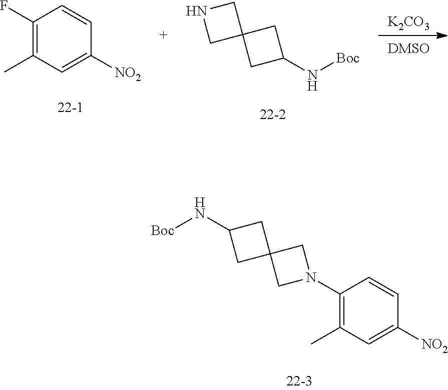

Following the same procedure as tert-butyl (7-(2-methyl-4-nitrophenyl)-7-azaspiro[3.5]nonan-2-yl)carbamate using tert-butyl (2-azaspiro[3.3]heptan-6-yl)carbamate instead of tert-butyl 7-azaspiro[3.5]nonan-2-ylcarbamate to obtain tert-butyl (2-(2-methyl-4-nitrophenyl)-2-azaspiro[3.3]heptan-6-yl)carbamate. MS: 348 [M+H]⁺

Step 2: Synthesis of tert-butyl(2-(4-amino-2-methylphenyl)-2-azaspiro[3.3]heptan-6-yl)carbamate

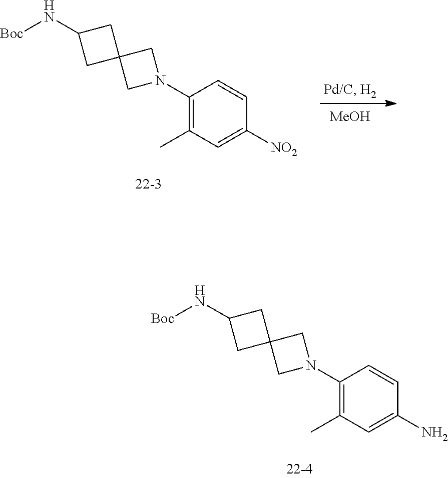 [SMILES: "MeOH" ; Cc1cc([N+](=O)[O-])ccc1N1CC2(CC(NC(=O)OC(C)(C)C)C2)C1 ; "Pd/C, H2" ; Cc1cc(N)ccc1N1CC2(CC(NC(=O)OC(C)(C)C)C2)C1]

Following the same procedure as tert-butyl(7-(4-amino-2-methylphenyl)-7-azaspiro[3.5]nonan-2-yl)carbamate using tert-butyl (2-(2-methyl-4-nitrophenyl)-2-azaspiro[3.3]heptan-6-yl)carbamate instead of tert-butyl (7-(2-methyl-4-nitrophenyl)-7-azaspiro[3.5]nonan-2-yl) carbamate to obtain tert-butyl (2-(4-amino-2-methylphenyl)-2-azaspiro[3.3]heptan-6-yl)carbamate. MS: 318 [M+H]⁺

Step 3: Synthesis of (2-((2-chloro-5-methylpyrimidin-4-yl)amino)phenyl)dimethylphosphine oxide

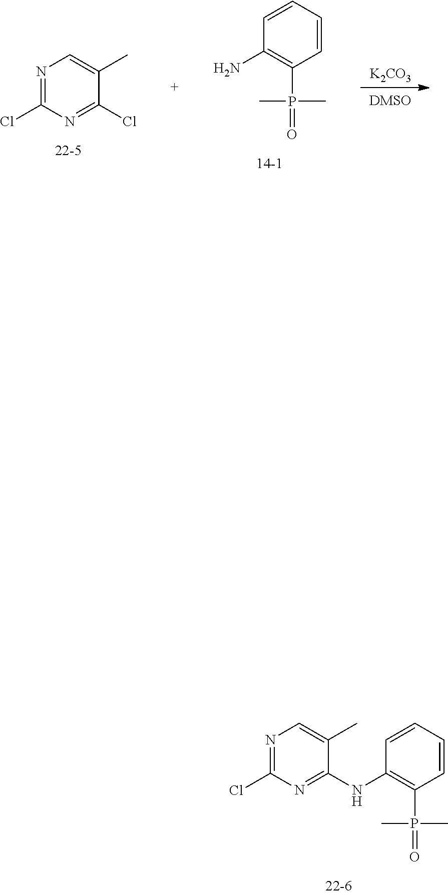

To a mixture of (2-aminophenyl)dimethylphosphine oxide (0.432 g) in DMF (10 mL), 2,4-dichloro-5-methylpyrimidine (0.500 g) and potassium carbonate (1.060 g) was added under stirring. The mixture was heated 100° C. for 24 h. The mixture solution was poured into water (30 ml) and extracted with ethyl acetate (30 mL*3). The combined organic layer was washed with water (100 ml*3) and saturated sodium chloride aqueous solution (100 mL), dried over Na₂SO₄ and concentrated under reduced pressure. The crude was purificated by Flash silica gel column chromatography (MeOH from 0% to 5%, 20 mins) and to obtain (2-((2-chloro-5-methylpyrimidin-4-yl)amino)phenyl)dimethylphosphine oxide(100 mg) as a white solid. MS: 296 [M+H]⁺.

Step 4: Synthesis of tert-butyl (2-(4-((4-((2-(dimethylphosphoryl)phenyl)amino)-5-methylpyrimidin-2-yl)amino)-2-methylphenyl)-2-azaspiro[3.3]heptan-6-yl)carbamate

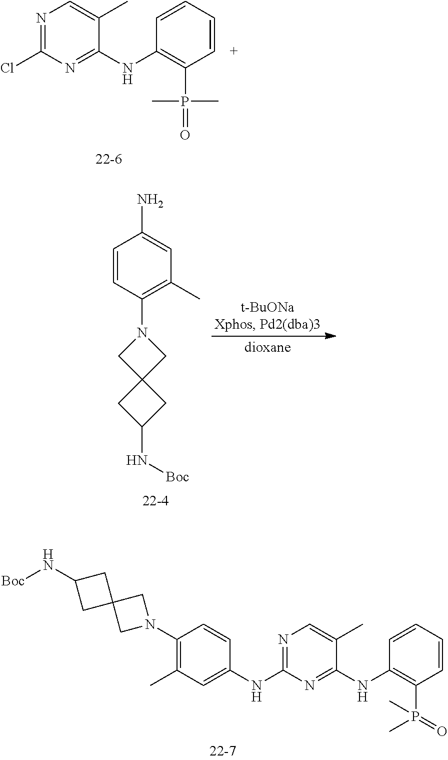 [SMILES: Cc1cnc(Cl)nc1Nc1ccccc1P(C)(C)=O ; Cc1cc(N)ccc1N1CC2(CC(NC(=O)OC(C)(C)C)C2)C1 ; Cc1cc(Nc2ncc(C)c(Nc3ccccc3P(C)(C)=O)n2)ccc1N1CC2(CC(NC(=O)OC(C)(C)C)C2)C1]

To a solution of (2-((2-chloro-5-methylpyrimidin-4-yl)amino)phenyl)dimethylphosphine oxide (59 mg) in dioxane (5 mL) was added tert-butyl (2-(4-amino-2-methylphenyl)-2-azaspiro[3.3]heptan-6-yl)carbamate (66 mg), Xphos (18 mg), Pd₂(dba)₃ (17 mg) and t-BuONa (54 mg). The mixture was charged with nitrogen, and heated 100° C. for about 12 h. The reaction mixture was concentrated and the crude was purificated by Flash silica gel column chromatography (MeOH from 0% to 5%, 20 mins) and to obtain tert-butyl (2-((4-((4-((2-(dimethylphosphoryl)phenyl)amino)-5-methylpyrimidin-2-yl)amino)-2-methylphenyl)-2-azaspiro[3.3]heptan-6-yl)carbamate (15 mg) as brown solid. MS: 577 [M+H]⁺.

Step 5: Synthesis of (2-((2-((4-(6-amino-2-azaspiro[3.3]heptan-2-yl)-3-methylphenyl)amino)-5-methylpyrimidin-4-yl)amino)phenyl)dimethylphosphine oxide trifluoroacetate

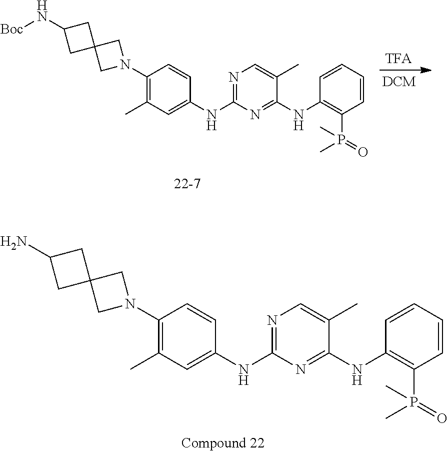 [SMILES: Cc1cc(Nc2ncc(C)c(Nc3ccccc3P(C)(C)=O)n2)ccc1N1CC2(CC(NC(=O)OC(C)(C)C)C2)C1 ; Cc1cc(Nc2ncc(C)c(Nc3ccccc3P(C)(C)=O)n2)ccc1N1CC2(CC(N)C2)C1]

To a solution of tert-butyl (2-(4-((4-((2-(dimethylphosphoryl)phenyl)amino)-5-methylpyrimidin-2-yl)amino)-2-methylphenyl)-2-azaspiro[3.3]heptan-6-yl)carbamate (15 mg) in DCM(3 ml) was added TFA (1 mL). The mixture was stirred 1 hr at room temperature. The resulting solution was concentrated under vaccum and the residue was beaten with n-hexane. After filtration, the solid was washed with n-hexane to obtain (2-((2-((4-(6-amino-2-azaspiro[3 . 3] heptan-2-yl)-3-methylphenyl)amino)-5-methyl pyrimidin-4-yl)amino)phenyl)dimethylphosphine oxide trifluoroacetate (12.5 mg) as white solid.

MS of Compound 22: 477 [M+H]⁺.

### Example 23

### Synthesis of Compound 23

### (2-((24(4-(6-amino-2-azaspiro[3.3]heptan-2-yl)-3-methylphenyl)amino)-5-methoxypyrimidin-4-yl)amino)phenyl)dimethylphosphine oxide

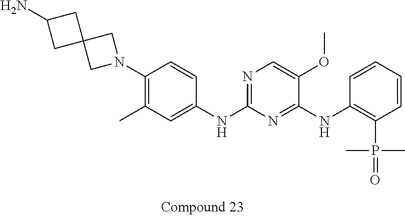 [SMILES: COc1cnc(Nc2ccc(N3CC4(CC(N)C4)C3)c(C)c2)nc1Nc1ccccc1P(C)(C)=O]

Step 1: Synthesis of (2-((2-chloro-5-methoxypyrimidin-4-yl)amino)phenyl)dimethylphosphine oxide

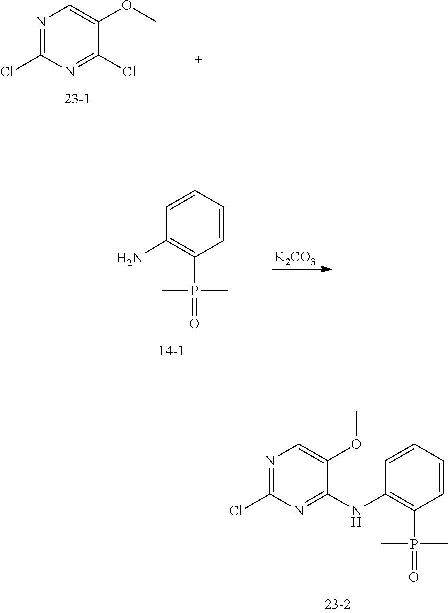

Following the same procedure as (2-((2-chloro-5-methylpyrimidin-4-yl)amino)phenyl)dimethylphosphine oxide using 2,4-dichloro-5-methoxypyrimidine instead of 2,4-dichloro-5-methylpyrimidine to obtain (2-((2-chloro-5-methoxypyrimidin-4-yl)amino)phenyl)dimethylphosphine oxide. MS: 312 [M+H]⁺

Step 2: Synthesis of tert-butyl(2-(4-((4-((2-(dimethylphosphoryl)phenyl)amino)-5-methoxypyrimidin-2-yl)amino)-2-methylphenyl)-2-azaspiro[3.3]heptan-6-yl)carbamate

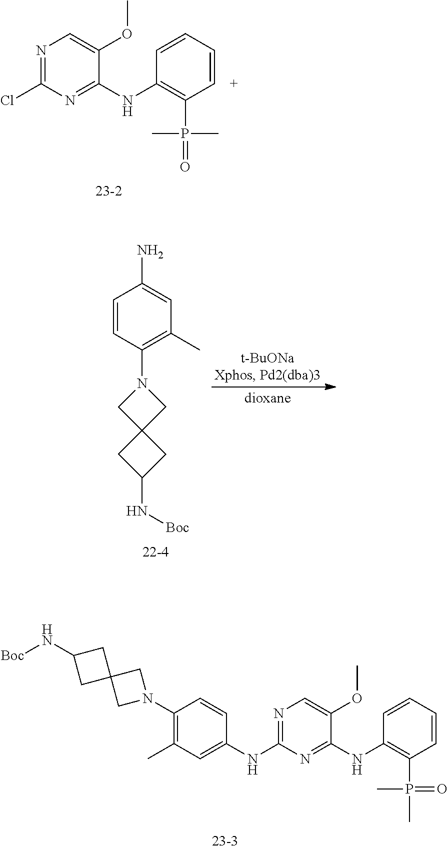 [SMILES: COc1cnc(Cl)nc1Nc1ccccc1P(C)(C)=O ; Cc1cc(N)ccc1N1CC2(CC(NC(=O)OC(C)(C)C)C2)C1 ; COc1cnc(Nc2ccc(N3CC4(CC(NC(=O)OC(C)(C)C)C4)C3)c(C)c2)nc1Nc1ccccc1P(C)(C)=O]

Following the same procedure as tert-butyl (7-(2-chloro-4-((4-((2-(dimethylphosphoryl)phenyl)amino)-5-methylpyrimidin-2-yl)amino)phenyl)-7-azaspiro[3.5]nonan-2-yl)carbamate using (2-((2-chloro-5-methoxypyrimidin-4-yl)amino)phenyl)dimethylphosphine oxide instead of (2-((2-chloro-5-methylpyrimidin-4-yl)amino)phenyl)dimethylphosphine oxide to obtain tert-butyl(2-(4-((4-((2-(dimethylphosphoryl)phenyl)amino)-5-methoxypyrimidin-2-yl)amino)-2-methylphenyl)-2-azaspiro[3.3]heptan-6-yl)carbamate. MS: 593 [M+H]⁺

Step 2: Synthesis of (2-((2-((4-(6-amino-2-azaspiro[3.3]heptan-2-yl)-3-methylphenyl)amino)-5-methoxypyrimidin-4-yl)amino)phenyl)dimethylphosphine oxide trifluoroacetate

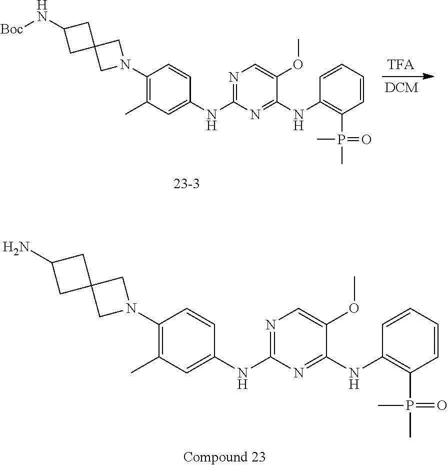 [SMILES: COc1cnc(Nc2ccc(N3CC4(CC(NC(=O)OC(C)(C)C)C4)C3)c(C)c2)nc1Nc1ccccc1P(C)(C)=O ; COc1cnc(Nc2ccc(N3CC4(CC(N)C4)C3)c(C)c2)nc1Nc1ccccc1P(C)(C)=O]

Following the same procedure as compound 22 using tert-butyl(2-(4-((4-((2-(dimethylphosphoryl)phenyl)amino)-5-methoxypyrimidin-2-yl)amino)-2-methylphenyl)-2-azaspiro[3.3]heptan-6-yl)carbamate instead of tert-butyl (2-(4-((4-((2-(dimethylphosphoryl)phenyl)amino)-5-methylpyrimidin-2-yl)amino)-2-methylphenyl)-2-azaspiro[3.3]heptan-6-yl)carbamate to obtain compound 23 (2-((2-((4-(6-amino-2-azaspiro[3.3]heptan-2-yl)-3-methylphenyl)amino)-5-methoxypyrimidin-4-yl)amino)phenyl)dimethylphosphine oxide trifluoroacetate. MS of Compound 23: 493 [M+H]⁺

### Example 24

### Synthesis of Compound 24

### (2-((2-((4-(6-amino-2-azaspiro[3.3]heptan-2-yl)-3-methylphenyl)amino)-5-fluoropyrimidin-4-yl)amino)phenyl)dimethylphosphine oxide

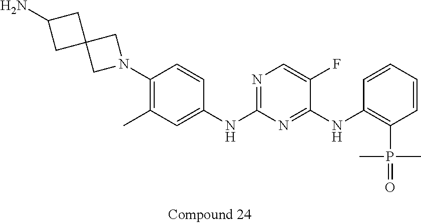 [SMILES: Cc1cc(Nc2ncc(F)c(Nc3ccccc3P(C)(C)=O)n2)ccc1N1CC2(CC(N)C2)C1]

Step 1: Synthesis of (2-((2-chloro-5-fluoropyrimidin-4-yl)amino)phenyl)dimethylphosphine oxide

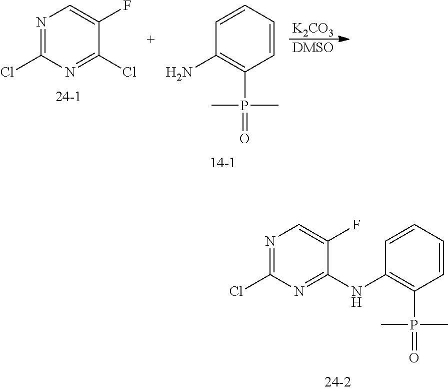

Following the same procedure as (2-((2-chloro-5-methylpyrimidin-4-yl)amino)phenyl)dimethylphosphine oxide using 2,4-dichloro-5-fluoropyrimidine instead of 2,4-dichloro-5-methylpyrimidine to obtain (2-((2-chloro -5-fluoropyrimidin-4-yl)amino)phenyl)dimethylphosphine oxide. MS: 300 [M+H]⁺

Step 2:Synthesis of tert-butyl(2-(4-((4-((2-(dimethylphosphoryl)phenyl)amino)-5-fluoropyrimidin-2-yl)amino)-2-methylphenyl)-2-azaspiro[3.3]heptan-6-yl)carbonate

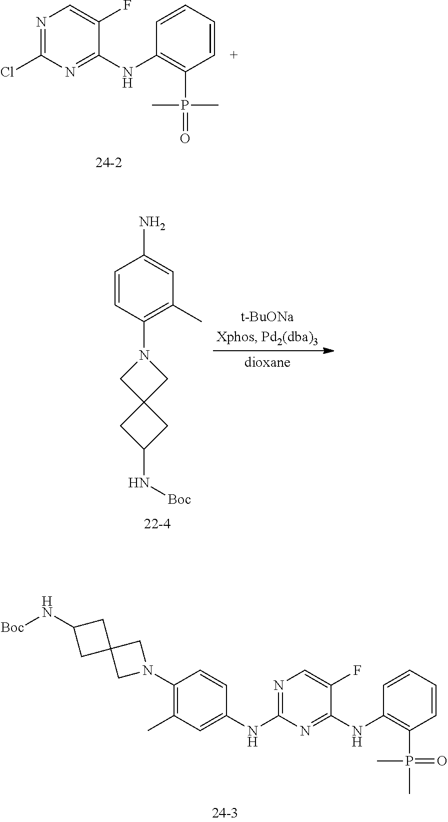 [SMILES: CP(C)(=O)c1ccccc1Nc1nc(Cl)ncc1F ; Cc1cc(N)ccc1N1CC2(CC(NC(=O)OC(C)(C)C)C2)C1 ; Cc1cc(Nc2ncc(F)c(Nc3ccccc3P(C)(C)=O)n2)ccc1N1CC2(CC(NC(=O)OC(C)(C)C)C2)C1]

Following the same procedure as tert-butyl(7-(2-chloro-4-((4-((2-(dimethylphosphoryl)phenyl)amino)-5-methylpyrimidin-2-yl)amino)phenyl)-7-azaspiro[3.5]nonan-2-yl)carbamate using (2-((2-chloro-5-fluoropyrimidin-4-yl)amino)phenyl)dimethylphosphine oxide instead of (2-((2-chloro-5-methylpyrimidin-4-yl)amino)phenyl)dimethylphosphine oxide to obtain (2-(4-((4-((2-(dimethylphosphoryl)phenyl)amino)-5-fluoropyrimidin-2-yl)amino)-2-methylphenyl)-2-azaspiro[3.3]heptan-6-yl) carbamate. MS: 581 [M+H]⁺.

Step 3: Synthesis of (2-((2-((4-(6-amino-2-azaspiro[3.3]heptan-2-yl)-3-methylphenyl)amino)-5-fluoropyrimidin-4-yl)amino)phenyl)dimethylphosphine oxide trifluoroacetate

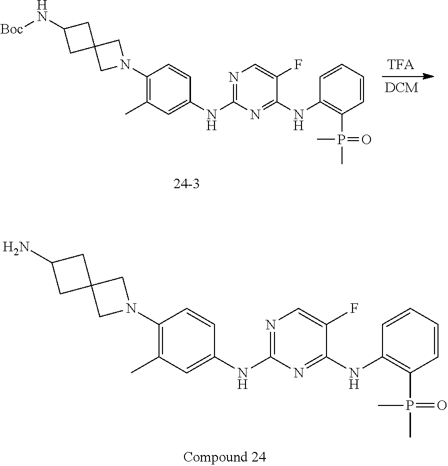 [SMILES: Cc1cc(Nc2ncc(F)c(Nc3ccccc3P(C)(C)=O)n2)ccc1N1CC2(CC(NC(=O)OC(C)(C)C)C2)C1 ; Cc1cc(Nc2ncc(F)c(Nc3ccccc3P(C)(C)=O)n2)ccc1N1CC2(CC(N)C2)C1]

Following the same procedure as compound 22 using tert-butyl(2-(4-((4-((2-(dimethylphosphoryl)phenyl)amino)-5-fluoropyrimidin-2-yl)amino)-2-methylphenyl)-2-azaspiro[3.3]heptan-6-yl)carbamate instead of tert-butyl (2-(4-((4-((2-(dimethylphosphoryl)phenyl)amino)-5-methylpyrimidin-2-yl)amino)-2-methylphenyl)-2-azaspiro[3.3]heptan-6-yl)carbamate to obtain (2-((2-((4-(6-amino-2-azaspiro[3.3]heptan-2-yl)-3-methylphenyl)amino)-5-fluoropyrimidin-4-yl)amino)phenyl)dimethylphosphine oxide trifluoroacetate. MS of Compound 24: 481 [M+H]⁺

### Example 25

### Synthesis of Compound 25

### (2-((2-((4-(6-amino -2-azaspiro[3.3]heptan-2-yl)-3-methylphenyl)amino)-5-chloropyrimidin-4-yl)amino)phenyl)dimethylphosphine oxide

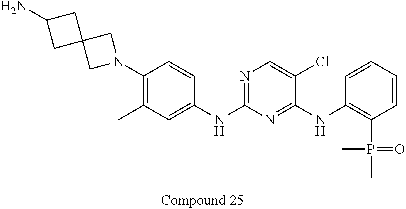 [SMILES: Cc1cc(Nc2ncc(Cl)c(Nc3ccccc3P(C)(C)=O)n2)ccc1N1CC2(CC(N)C2)C1]

Step 1: Synthesis of tert-butyl(2-(4-((5-chloro-4-((2-(dimethylphosphoryl)phenyl)amino)pyrimidin-2-yl)amino)-2-methylphenyl)-2-azaspiro[3.3]heptan-6-yl)carbamate

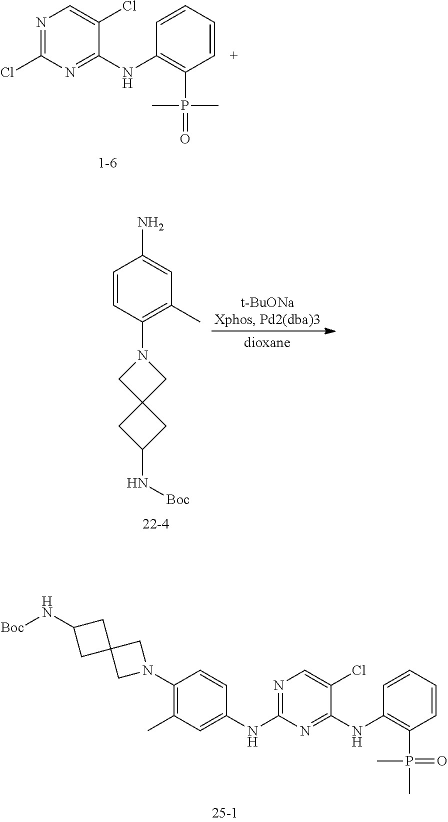 [SMILES: CP(C)(=O)c1ccccc1Nc1nc(Cl)ncc1Cl ; Cc1cc(N)ccc1N1CC2(CC(NC(=O)OC(C)(C)C)C2)C1 ; Cc1cc(Nc2ncc(Cl)c(Nc3ccccc3P(C)(C)=O)n2)ccc1N1CC2(CC(NC(=O)OC(C)(C)C)C2)C1]

Following the same procedure as tert-butyl(7-(2-chloro-4-((4-((2-(dimethylphosphoryl)phenyl)amino)-5-methylpyrimidin-2-yl)amino)phenyl)-7-azaspiro[3.5]nonan-2-yl)carbamate using (2-((2,5-dichloropyrimidin-4-yl)amino)phenyl)dimethylphosphine oxide instead of (2-((2-chloro-5-methylpyrimidin-4-yl)amino)phenyl)dimethylphosphine oxide to obtain tert-butyl(2-(4-((5-chloro-4-((2-(dimethylphosphoryl)phenyl)amino)pyrimidin-2-yl)amino)-2-methylphenyl)-2-azaspiro[3.3]heptan-6-yl)carbamate. MS: 597 [M+H]⁺.

Step 2: Synthesis of (2-((2-((4-(6-amino-2-azaspiro[3.3]heptan-2-yl)-3-methylphenyl)amino)-5-chloropyrimidin-4-yl)amino)phenyl)dimethylphosphine oxide

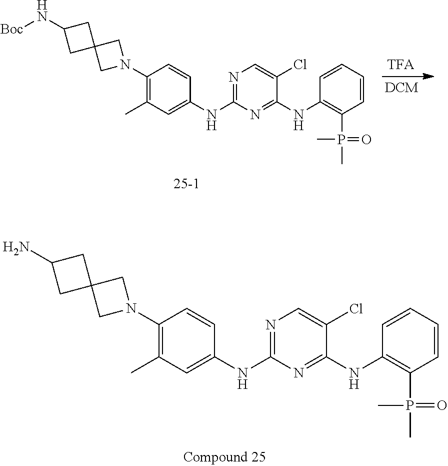 [SMILES: Cc1cc(Nc2ncc(Cl)c(Nc3ccccc3P(C)(C)=O)n2)ccc1N1CC2(CC(NC(=O)OC(C)(C)C)C2)C1 ; Cc1cc(Nc2ncc(Cl)c(Nc3ccccc3P(C)(C)=O)n2)ccc1N1CC2(CC(N)C2)C1]

Following the same procedure as compound 22 using tert-butyl (2-(4-((5-chloro-4-((2-(dimethylphosphoryl)phenyl)amino)pyrimidin-2-yl)amino)-2-methylphenyl)-2-azaspiro[3.3]heptan-6-yl)carbamate instead of tert-butyl (2-(4-((4-((2-(dimethylphosphoryl)phenyl)amino)-5-methylpyrimidin-2-yl)amino)-2-methylphenyl)-2-azaspiro[3.3]heptan-6-yl)carbamate. Then the product was dissolved in MeOH and the PH of resouting solution was adjusted to 8-9 with sodium carbonate aqueous solution. The resulting mixture was concentrated and the residue was purificated by flash silica gel column(The ratio of methanol changes from 0 to 10%) to obtain (2-((2-((4-(6-amino-2-azaspiro[3.3]heptan-2-yl)-3-methylphenyl)amino-5-chloropyrimidin-4-yl)amino)phenyl)dimethylphosphine oxide. MS of Compound 25: 497 [M+H]⁺

### Example 26

### Synthesis of Compound 26

### (2-((2-((4-(6-amino-2-azaspiro[3.3]heptan-2-yl)-3-chlorophenyl)amino)-5-chloropyrimidin-4-yl)amino)phenyl)dimethylphosphine oxide

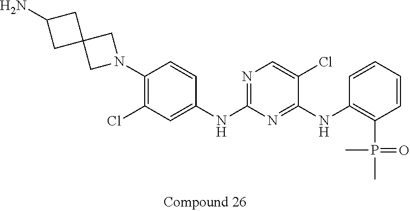 [SMILES: CP(C)(=O)c1ccccc1Nc1nc(Nc2ccc(N3CC4(CC(N)C4)C3)c(Cl)c2)ncc1Cl]

Step 1: Synthesis of tert-butyl(2-(2-chloro-4-nitrophenyl)-2-azaspiro[3.3]heptan-6-yl)carbamate

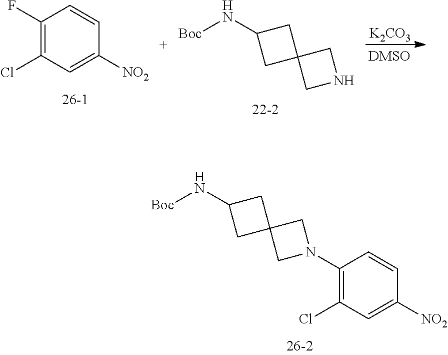

Following the same procedure as tert-butyl (7-(2-methyl-4-nitrophenyl)-7-azaspiro[3.5]nonan-2-yl)carbamate using tert-butyl (2-azaspiro[3.3]heptan-6-yl)carbamate instead of tert-butyl 7-azaspiro[3.5]nonan-2-ylcarbamate and using 2-chloro-1-fluoro-4-nitrobenzene instead of 2-fluoro-5-nitrotoluene, to obtain tert-butyl (2-(2-methyl-4-nitrophenyl)-2-azaspiro[3.3]heptan-6-yl)carbamate. MS: 368 [M+H]⁺.

Step 2: Synthesis of tert-butyl(2-(4-amino-2-chlorophenyl)-2-azaspiro[3.3]heptan-6-yl)carbamate

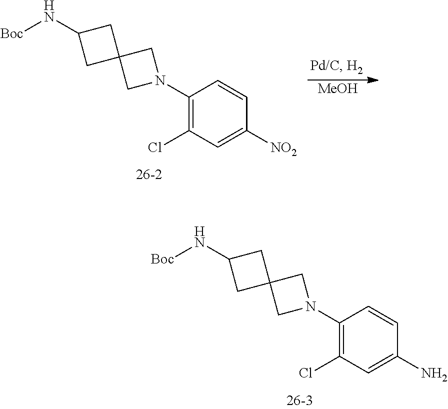 [SMILES: "MeOH" ; CC(C)(C)OC(=O)NC1CC2(C1)CN(c1ccc([N+](=O)[O-])cc1Cl)C2 ; CC(C)(C)OC(=O)NC1CC2(C1)CN(c1ccc(N)cc1Cl)C2]

Following the same procedure as tert-butyl (7-(4-amino-2-methylphenyl)-7-azaspiro[3.5]nonan-2-yl)carbamate using tert-butyl (2-(2-chloro-4-nitrophenyl)-2-azaspiro[3.3]heptan-6-yl)carbamate instead of tert-butyl (7-(2-methyl-4-nitrophenyl)-7-azaspirto[3.5]nonan-2-yl) carbamate to obtain tert-butyl(2-(4-amino-2-chlorophenyl)-2-azaspiro[3.3]heptan-6-yl)carbamate. MS: 338 [M+H]⁺

Step 2: Synthesis of tert-butyl(2-(2-chloro-4-((5-chloro-4-((2-(dimethylphosphoryl)phenyl)amino)pyrimidin-2-yl)amino)phenyl)-2-azaspiro[3.3]heptan-6-yl)carbamate

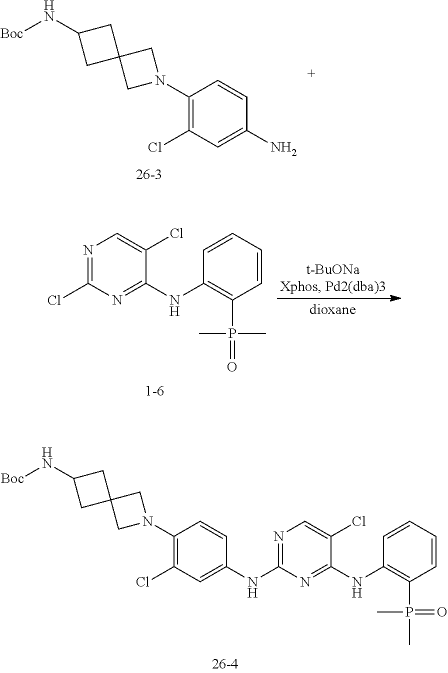 [SMILES: CC(C)(C)OC(=O)NC1CC2(C1)CN(c1ccc(N)cc1Cl)C2 ; CP(C)(=O)c1ccccc1Nc1nc(Cl)ncc1Cl ; CC(C)(C)OC(=O)NC1CC2(C1)CN(c1ccc(Nc3ncc(Cl)c(Nc4ccccc4P(C)(C)=O)n3)cc1Cl)C2]

Following the same procedure as tert-butyl(7-(4-((5-chloro-4-((2-(dimethylphosphoryl)phenyl)amino)pyrimidin-2-yl)amino)-2-methylphenyl)-7-azaspiro[3.5]nonan-2-yl)carbamate using tert-butyl(2-(4-amino-2-chlorophenyl)-2-azaspiro[3.3]heptan-6-yl)carbamate instead of (2-((2-chloro-5-methylpyrimidin-4-yl)amino)phenyl)dimethylphosphine oxide to obtain tert-butyl(2-(2-chloro-4-((5-chloro-4-((2-(dimethylphosphoryl)phenyl)amino)pyrimidin-2-yl)amino)phenyl)-2-azaspiro[3.3]heptan-6-yl)carbamate. MS: 617 [M+H]⁺

Step 4: Synthesis of (2-((2-((4-(6-amino-2-azaspiro[3.3]heptan-2-yl)-3-chlorophenyl)amino)-5-chloropyrimidin-4-yl)amino)phenyl)dimethylphosphine oxide

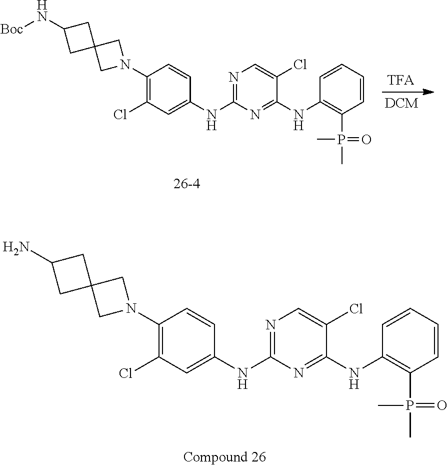 [SMILES: CC(C)(C)OC(=O)NC1CC2(C1)CN(c1ccc(Nc3ncc(Cl)c(Nc4ccccc4P(C)(C)=O)n3)cc1Cl)C2 ; CP(C)(=O)c1ccccc1Nc1nc(Nc2ccc(N3CC4(CC(N)C4)C3)c(Cl)c2)ncc1Cl]

Following the same procedure as compound 22 using tert-butyl (2-(2-chloro-4-((5-chloro-4-((2-(dimethylphosphoryl)phenyl)amino)pyrimidin-2-yl)amino)phenyl)-2-azaspiro[3.3]heptan-6-yl)carbamate instead of tert-butyl (2-(4-((4-((2-(dimethylphosphoryl)phenyl)amino)-5-methylpyrimidin-2-yl)amino)-2-methylphenyl)-2-azaspiro[3.3]heptan-6-yl)carbamate. Then the product was dissolved in MeOH and the PH of resouting solution was adjusted to 8-9 with sodium carbonate aqueous solution. The resulting mixture was concentrated and the residue was purificated by flash silica gel column(The ratio of methanol changes from 0 to 10%) to obtain (2-((2-((4-(6-amino-2-azaspiro[3.3]heptan-2-yl)-3-chlorophenyl)amino)-5-chloropyrimidin-4-yl)amino)phenyl)dimethylphosphine oxide. MS of Compound 26: 517 [M+H]⁺

¹H NMR (500 MHz, MeOD) δ 8.417 (s, 1H), 8.053 (s, 1H), 7.626-7.555 (m, 3H), 7.260-7.215 (m, 2H), 6.561 (d, 1H, J=8.5 Hz), 4.002 (s, 2H),3.914 (s, 2H) 3.686-3.654 (m, 1H) 2.656-2.614 (m, 2H) 2.318-2.275 (m, 2H) 1.858 (m, 6H).

### Example 27

### Synthesis of Compound 27

### (2-((2-((4-(2-amino-7-azaspiro[3.5]nonan-7-yl)-3-methylphenyl)amino)-5-methoxypyrimidin-4-yl)amino)phenyl)dimethylphosphine oxide

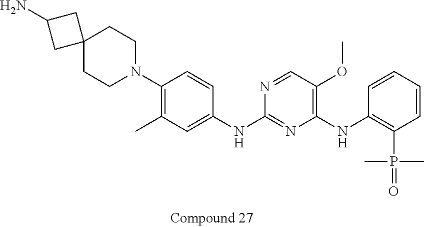 [SMILES: COc1cnc(Nc2ccc(N3CCC4(CC3)CC(N)C4)c(C)c2)nc1Nc1ccccc1P(C)(C)=O]

Step 1: Synthesis of tert-butyl7-(4-(4-(2-(dimethylphosphoryl)phenylamino)-5-methoxypyrimidin-2-ylamino)-2-methylphenyl)-7-azaspiro[3.5]nonan-2-ylcarbamate

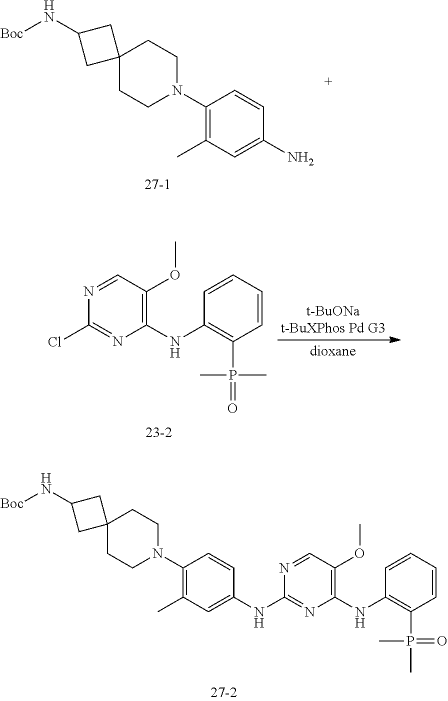 [SMILES: Cc1cc(N)ccc1N1CCC2(CC1)CC(NC(=O)OC(C)(C)C)C2 ; COc1cnc(Cl)nc1Nc1ccccc1P(C)(C)=O ; COc1cnc(Nc2ccc(N3CCC4(CC3)CC(NC(=O)OC(C)(C)C)C4)c(C)c2)nc1Nc1ccccc1P(C)(C)=O]

To a solution of tert-butyl (7-(4-amino-2-methylphenyl)-7-azaspiro[3.5]nonan-2-yl)carbamate (74 mg,), (2-((2-chloro-5-methoxypyrimidin-4-yl)amino)phenyl)dimethylphosphine oxide (80 mg), in dioxane (5 mL) with an inert atmosphere of nitrogen was added t-BuONa (62 mg), t-BuXPhos Pd G3(20 mg). The resulting solution was stirred for 16 h at 100° C. The resulting mixture was concentrated under reduced pressure. The residue was diluted with 50 mL of dichloromethane. The organic layer was washed with 25 mL of water, 25 mL of saturated sodium chloride respectively. The organic layer was dried over anhydrous sodium sulfate, filtered, and concentrated under reduced pressure. The residue was applied onto a silica gel column with dichloromethane/methanol (100:1-50:1). This obtained 30 mg (22%) of the desired product as white solid. MS: 621 [M+H]⁺

Step 2: Synthesis of (2-((2-((4-(2-amino-7-azaspiro[3.5]nonan-7-yl)-3-methylphenyl)amino)-5-methoxypyrimidin-4-yl)amino)phenyl)dimethylphosphine oxide trifluoroacetate

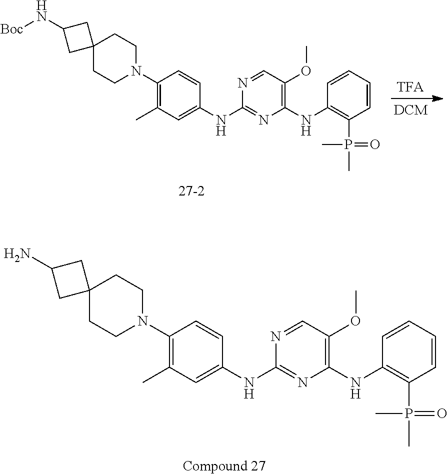 [SMILES: COc1cnc(Nc2ccc(N3CCC4(CC3)CC(NC(=O)OC(C)(C)C)C4)c(C)c2)nc1Nc1ccccc1P(C)(C)=O ; COc1cnc(Nc2ccc(N3CCC4(CC3)CC(N)C4)c(C)c2)nc1Nc1ccccc1P(C)(C)=O]

To a solution of tert-butyl7-(4-(4-(2-(dimethylphosphoryl)phenylamino)-5-methoxypyrimidin-2-ylamino)-2-methylphenyl)-7-azaspiro[3.5]nonan-2-ylcarbamate (30 mg) in dichloromethane (2 mL) was added trifluoroacetic acid (1 mL). The resulting solution was stirred for 1 h at 25° C. . The resulting mixture was concentrated under reduced pressure. This obtained 25 mg of the desired product as solid. MS of Compound 27: 521 [M+H]⁺

### Example 28

### Synthesis of Compound 28

### 2-((4-(2-amino-7-azaspiro[3.5]nonan-7-yl)-3-methylphenyl)amino)-4-((2-(dimethylphosphoryl)phenyl)amino)pyrimidine-5-carbonitrile

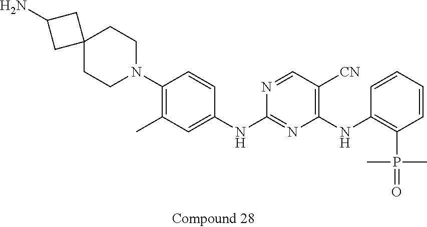 [SMILES: Cc1cc(Nc2ncc(C#N)c(Nc3ccccc3P(C)(C)=O)n2)ccc1N1CCC2(CC1)CC(N)C2]

Step 1: Synthesis of tert-butyl(7-(4-((5-cyano-4-((2-(dimethylphosphoryl)phenyl)amino)pyrimidin-2-yl)amino)-2-methylphenyl)-7-azaspiro[3.5]nonan-2-yl)carbamate

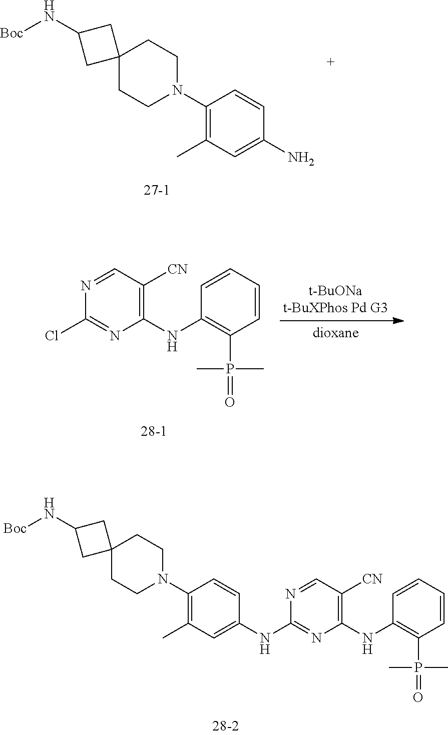 [SMILES: Cc1cc(N)ccc1N1CCC2(CC1)CC(NC(=O)OC(C)(C)C)C2 ; CP(C)(=O)c1ccccc1Nc1nc(Cl)ncc1C#N ; Cc1cc(Nc2ncc(C#N)c(Nc3ccccc3P(C)(C)=O)n2)ccc1N1CCC2(CC1)CC(NC(=O)OC(C)(C)C)C2]

To a solution of tert-butyl (7-(4-amino-2-methylphenyl)-7-azaspiro[3.5]nonan-2-yl)carbamate (75 mg), (2-((2-chloro-5-methoxypyrimidin-4-yl)amino)phenyl)dimethylphosphine oxide (80 mg), in dioxane (5 mL) with an inert atmosphere of nitrogen was added t-BuONa (63 mg), t-BuXPhos Pd G3(20 mg). The resulting solution was stirred for 16 h at 100° C. The resulting mixture was concentrated under reduced pressure. The residue was diluted with 50 mL of dichloromethane. The organic layer was washed with 25 mL of water, 25 mL of saturated sodium chloride respectively. The organic layer was dried over anhydrous sodium sulfate, filtered, and concentrated under reduced pressure. The residue was applied onto a silica gel column with dichloromethane/methanol (100:1-40:1). This obtained 82 mg (61%) of the desired product as white solid. MS: 616 [M+H]⁺

Step 2: Synthesis of 2-(4-(2-amino-7-azaspiro[3.5]nonan-7-yl)-3-methylphenylamino)-4-(2-(dimethylphosphoryl)phenylamino)pyrimidine-5-carbonitrile trifluoroacetate

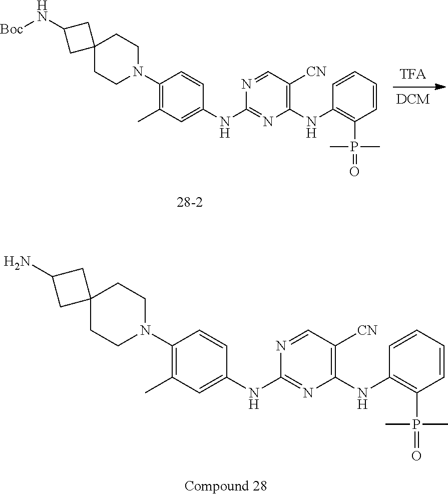 [SMILES: Cc1cc(Nc2ncc(C#N)c(Nc3ccccc3P(C)(C)=O)n2)ccc1N1CCC2(CC1)CC(NC(=O)OC(C)(C)C)C2 ; Cc1cc(Nc2ncc(C#N)c(Nc3ccccc3P(C)(C)=O)n2)ccc1N1CCC2(CC1)CC(N)C2]

To a solution of tert-butyl (7-(4-((5-cyano-4-((2-(dimethylphosphoryl)phenyl)amino)pyrimidin-2-yl)amino)-2-methylphenyl)-7-azaspiro[3.5]nonan-2-yl)carbamate (82 mg) in dichloromethane (2 mL) was added trifluoroacetic acid (1 mL). The resulting solution was stirred for 1 h at 25 L. The resulting mixture was concentrated under reduced pressure. This obtained 70 mg (90%) of the desired product as solid. MS of Compound 28: 516 [M+H]⁺

### Example 29

### Synthesis of Compound 29

### (2-((24(4-(2-amino-7-azaspiro[ 3.5]nonan-7-yl)-3-fluorophenyl)amino)-5-chloropyrimidin-4-yl)amino)phenyl)dimethylphosphine oxide

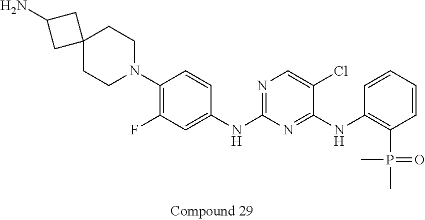 [SMILES: CP(C)(=O)c1ccccc1Nc1nc(Nc2ccc(N3CCC4(CC3)CC(N)C4)c(F)c2)ncc1Cl]

Step 1: Synthesis of tert-butyl (7-(2-fluoro-4-nitrophenyl)-7-azaspiro[3.5]nonan-2-yl)carbamate

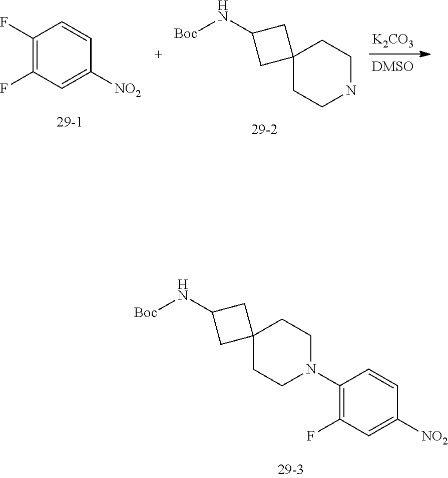

Following the same procedure as tert-butyl (7-(2-methyl-4-nitrophenyl)-7-azaspiro[3.5]nonan-2-yl)carbamate using 1,2-difluoro-4-nitrobenzene instead of 2-fluoro-5-nitrotoluene to obtain tert-butyl (7-(2-fluoro-4-nitrophenyl)-7-azaspiro[3.5]nonan-2-yl)carbamate. MS: 380 [M+H]⁺.

Step 2: Synthesis of tert-butyl (7-(4-amino-2-fluorophenyl)-7-azaspiro[3.5]nonan-2-yl)carbamate

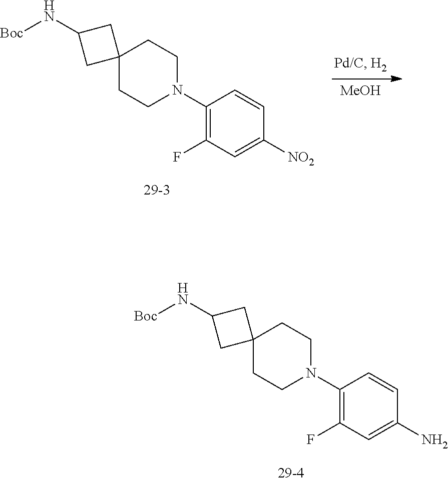 [SMILES: "MeOH" ; CC(C)(C)OC(=O)NC1CC2(CCN(c3ccc([N+](=O)[O-])cc3F)CC2)C1 ; CC(C)(C)OC(=O)NC1CC2(CCN(c3ccc(N)cc3F)CC2)C1]

Following the same procedure as tert-butyl(7-(4-amino-2-methylphenyl)-7-azaspiro[3.5]nonan-2-yl)carbamate using tert-butyl (7-(2-fluoro-4-nitrophenyl)-7-azaspiro[3.5]nonan-2-yl)carbamate instead of tert-butyl (7-(2-methyl-4-nitrophenyl)-7-azaspiro[3.5]nonan-2-yl)carbamate to obtain tert-butyl (7-(4-amino-2-fluorophenyl)-7-azaspiro[3.5]nonan-2-yl)carbamate. MS: 350 [M+H]⁺

Step 2: Synthesis of tert-butyl (7-(4-((5-chloro-4-((2-(dimethylphosphoryl)phenyl)amino)pyrimidin-2-yl)amino)-2-fluorophenyl)-7-azaspiro[3.5]nonan-2-yl)carbamate

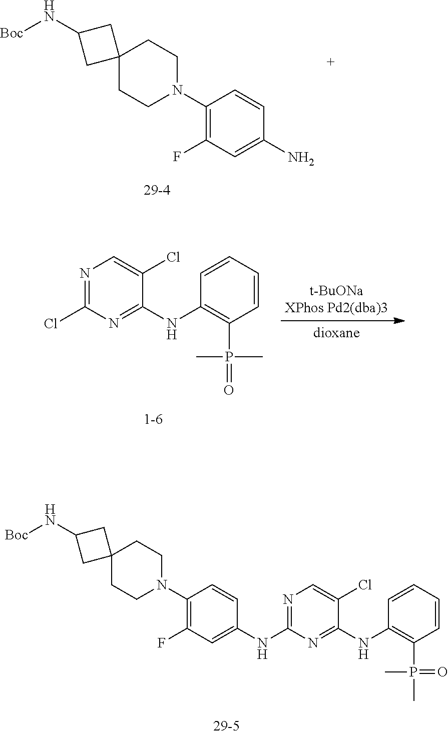 [SMILES: CC(C)(C)OC(=O)NC1CC2(CCN(c3ccc(N)cc3F)CC2)C1 ; CP(C)(=O)c1ccccc1Nc1nc(Cl)ncc1Cl ; CC(C)(C)OC(=O)NC1CC2(CCN(c3ccc(Nc4ncc(Cl)c(Nc5ccccc5P(C)(C)=O)n4)cc3F)CC2)C1]

Following the same procedure as tert-butyl(7-(4-((5-chloro-4-((2-(dimethylphosphoryl)phenyl)amino)pyrimidin-2-yl)amino)-2-methylphenyl)-7-azaspiro[3.5]nonan-2-yl)carbamate using tert-butyl (7-(4-amino-2-fluorophenyl)-7-azaspiro[3.5]nonan-2-yl)carbamate instead of tert-butyl(7-(4-amino-2-methylphenyl)-7-azaspiro[3.5]nonan-2-yl)carbamate to obtain tert-butyl(7-(4-((5-chloro-4-((2-(dimethylphosphoryl)phenyl)amino)pyrimidin-2-yl)amino)-2-fluorophenyl)-7-azaspiro[3.5]nonan-2-yl)carbamate. MS: 629 [M+H]⁺

Step 3: Synthesis of (2-((2-((4-(2-amino-7-azaspiro[3.5]nonan-7-yl)-3-fluorophenyl)amino)-5-chloropyrimidin-4-yl)amino)phenyl)dimethylphosphine oxide trifluoroacetate

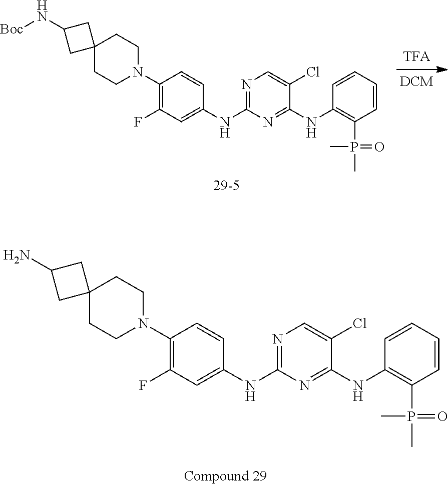 [SMILES: CC(C)(C)OC(=O)NC1CC2(CCN(c3ccc(Nc4ncc(Cl)c(Nc5ccccc5P(C)(C)=O)n4)cc3F)CC2)C1 ; CP(C)(=O)c1ccccc1Nc1nc(Nc2ccc(N3CCC4(CC3)CC(N)C4)c(F)c2)ncc1Cl]

Following the same procedure as compound 22 using tert-butyl (7-(4-((5-chloro-4-((2-(dimethylphosphoryl)phenyl)amino)pyrimidin-2-yl)amino)-2-fluorophenyl)-7-azaspiro[3.5]nonan-2-yl)carbamate instead of tert-butyl (2-(4-((4-((2-(dimethylphosphoryl)phenyl)amino)-5-methylpyrimidin-2-yl)amino)-2-methylphenyl)-2-azaspiro[3.3]heptan-6-yl)carbamate to obtain (2-((2-((4-(2-amino-7-azaspiro[3.5]nonan-7-yl)-3-fluorophenyl)amino)-5-chloropyrimidin-4-yl)amino)phenyl)dimethylphosphine oxide trifluoroacetate. MS of Compound 29: 529 [M+H]⁺.

### Example 30

### Synthesis of Compound 30

### (2-((5-chloro-2-((4-(2-(dimethylamino)-7-azaspiro[3.5]nonan-7-yl)-3-fluorophenyl)amino)pyrimidin-4-yl)amino)phenyl)dimethylphosphine oxide

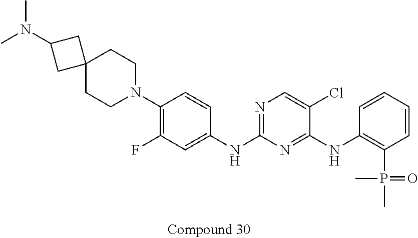 [SMILES: CN(C)C1CC2(CCN(c3ccc(Nc4ncc(Cl)c(Nc5ccccc5P(C)(C)=O)n4)cc3F)CC2)C1]

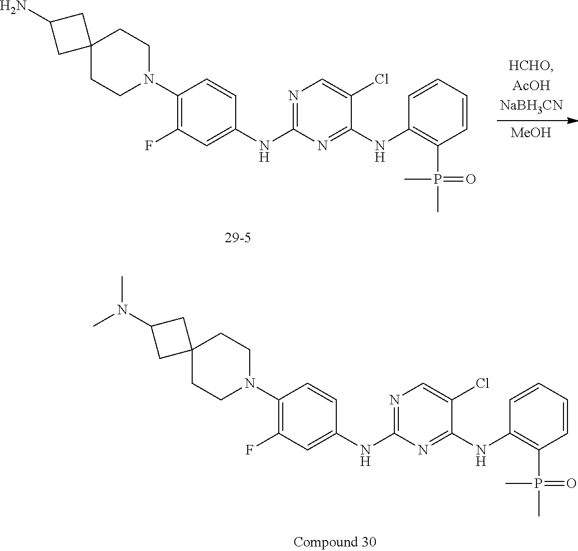 [SMILES: "MeOH" ; CP(C)(=O)c1ccccc1Nc1nc(Nc2ccc(N3CCC4(CC3)CC(N)C4)c(F)c2)ncc1Cl ; CN(C)C1CC2(CCN(c3ccc(Nc4ncc(Cl)c(Nc5ccccc5P(C)(C)=O)n4)cc3F)CC2)C1]

In 50 ml single-mouth bottle, to a solution of (2-((2-((4-(2-amino-7-azaspiro[3.5]nonan-7-yl)-3-fluorophenyl)amino)-5-chloropyrimidin-4-yl)amino)phenyl)dimethylphosphine oxide trifluoroacetate (160 mg) in methanol (10 mL) was added formaldehyde solution (36 mg). The resulting solution was stirred 1 hr at 30° C. Then the reaction mixture was added NaBH₃CN (42 mg), and stirred another 2 hrs. The reaction mixture was concentrated, and the residue was purified by silica gel column chromatography using DCM/methanol (20:1) as the eluent. And to obtain compound 30 (2-((5-chloro-2-((4-(2-(dimethylamino)-7-azaspiro[3.5]nonan-7-yl)-3-fluorophenyl)amino)pyrimidin-4-yl)amino)phenyl)dimethylphosphine oxide. MS of Compound 30: 557 [M+H]⁺.

¹H NMR (500 MHz, MeOD) δ 8.404 (s, 1H), 8.087 (s, 1H), 7.654-7.570 (m, 2H), 7.525-7.496 (m, 1H), 7.299-7.285 (m, 1H), 7.116-7.099 (m, 1H), 6.948-6.912 (m, 1H), 3.711-3.679(m, 1H), 2.973-2.905 (m, 4H) 2.788 (s, 6H) 2.378 (s, 2H) 1.981-1.963 (m, 2H), 1.856-1.790 (m, 6H), 1.291 (s, 4 H).

### Example 31

### Synthesis of Compound 31

### (2-((5-chloro-2-((3-chloro-4-(2-(cyclopentylamino)-7-azaspiro[3.5]nonan-7-yl)phenyl)amino)pyrimidin-4-yl)amino)phenyl)dimethylphosphine oxide

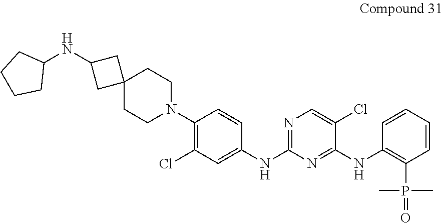 [SMILES: CP(C)(=O)c1ccccc1Nc1nc(Nc2ccc(N3CCC4(CC3)CC(NC3CCCC3)C4)c(Cl)c2)ncc1Cl]

Step 1: Synthesis of 7-(2-chloro-4-nitrophenyl)-7-azaspiro[3.5]nonan-2-one

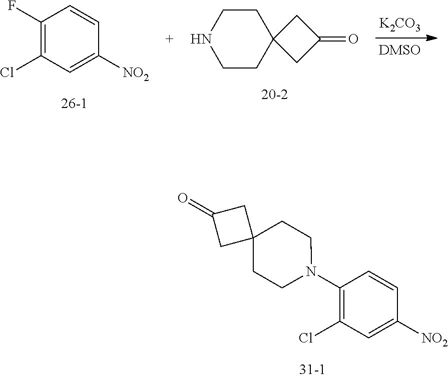

In 100 ml single-mouth bottle, to a solution of 2-chloro-1-fluoro-4-nitrobenzene(2.00 g) in DMSO(50 ml), was added 7-azaspiro[3.5]nonan-2-one(2.20 g) and anhydrous potassium carbonate(4.72 g). The resulting solution was stirred for 6 h at 90° C. The resulting mixture was diluted with 100 mL of water and 100 ml of ethyl acetate. Stratification, collection of organic phase. The organic layer was washed with water(100 ml*3) and saturated sodium chloride aqueous solution (100 mL), dried over Na₂SO₄ and concentrated to give crude product, which was purified by silica gel column chromatography using hexane/ethyl acetate (4: 1) as the eluent, and to obtain 7-(2-chloro-4-nitrophenyl)-7-azaspiro[3.5]nonan-2-one as yellow solid. MS: 295 [M+H]⁺.

Step 2: Synthesis of 7-(4-amino-2-chlorophenyl)-7-azaspiro[3.5]nonan-2-one

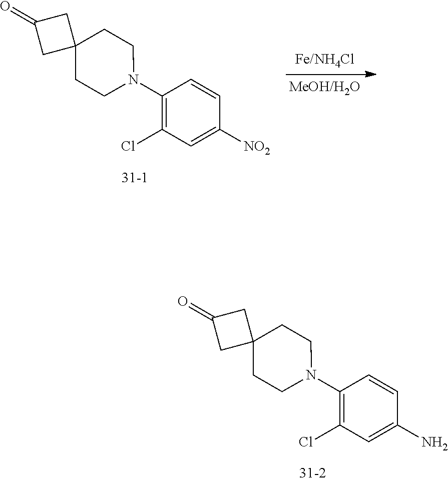 [SMILES: O=C1CC2(CCN(c3ccc([N+](=O)[O-])cc3Cl)CC2)C1 ; Nc1ccc(N2CCC3(CC2)CC(=O)C3)c(Cl)c1]

In 250 ml single-mouth bottle, to a solution of 7-(2-chloro-4-nitrophenyl)-7-azaspiro[3.5]nonan-2-one (2.85 g) in MeOH/H₂O (100 ml/10 ml), was added Fe (5.40 g) and NH₄Cl (5.17 g). The resulting solution was stirred for 4 h at 80° C. The resulting mixture was cooled to room temperature The mixture was then filtered through diatomaceous earth and washed with methanol; the filtrate was then concentrated under reduced pressure to afford the 7-(4-amino-2-chlorophenyl)-7-azaspiro[3.5]nonan-2-one as white solid. MS: 265 [M+H]⁺.

### Step 3: Synthesis of 7-(2-chloro-4-((5-chloro-4-((2-(dimethylphosphoryl)phenyl)amino)pyrimidin-2-yl)amino)phenyl)-7-azaspiro[3.5]nonan-2-one

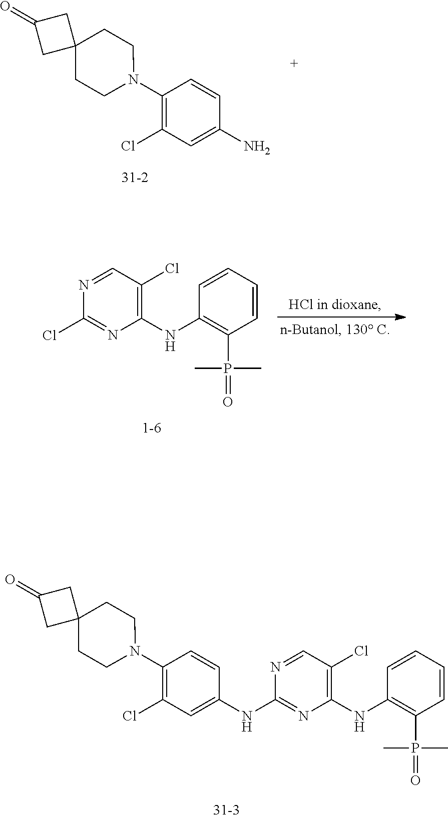 [SMILES: Nc1ccc(N2CCC3(CC2)CC(=O)C3)c(Cl)c1 ; CP(C)(=O)c1ccccc1Nc1nc(Cl)ncc1Cl ; CP(C)(=O)c1ccccc1Nc1nc(Nc2ccc(N3CCC4(CC3)CC(=O)C4)c(Cl)c2)ncc1Cl]

In 20 ml microwave tube, to a solution of 7-(4-amino-2-chlorophenyl)-7-azaspiro[3.5]nonan-2-one (300 mg) and (2-((2,5-dichloropyrimidin-4-yl)amino)phenyl)dimethylphosphine oxide (358 mg) in n-Butanol(10 ml), was added HCl in 1,4-dioxane (0.56 ml). The resulting solution was microwaved at 130° C. for 2 hours. The resulting mixture was concentrated under vaccum. The residue was purificated by silica gel column chromatography using DCM/methanol (20: 1) as the eluent. And to obtain 7-(2-chloro-4-((5-chloro-4-((2-(dimethylphosphoryl)phenyl)amino)pyrimidin-2-yl)amino)phenyl)-7-azaspiro[3.5]nonan-2-one (54.5 mg) as brown solid.

Step 4: Synthesis of (2-((5-chloro-2-((3-chloro-4-(2-(cyclopentylamino)-7-azaspiro[3.5]nonan-7-yl)phenyl)amino)pyrimidin-4-yl)amino)phenyl)dimethylphosphine oxide

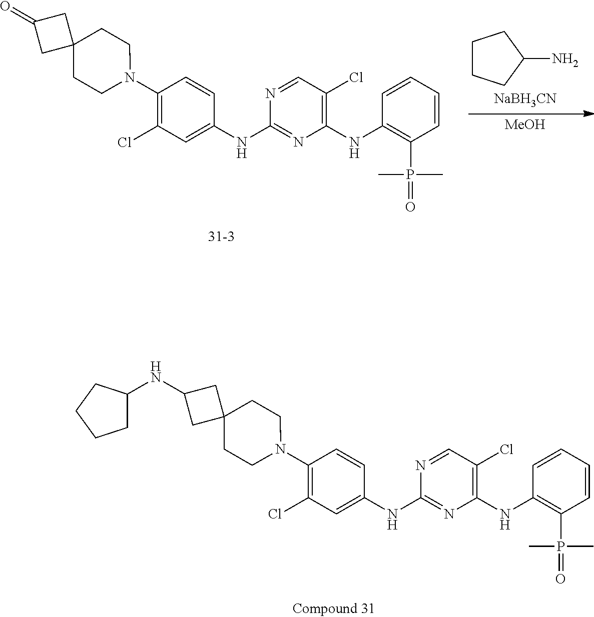

In 50 ml single-mouth bottle, to a solution of 7-(2-chloro-4-((5-chloro-4-((2-(dimethylphosphoryl)phenyl)amino)pyrimidin-2-yl)amino)phenyl)-7-azaspiro[3.5]nonan-2-one hydrochloric acid salt (120 mg) in methanol (10 mL) was added cyclopentanamine (36 mg). The resulting solution was stirred 30 min at 30° C. Then the reaction mixture was added NaBH₃CN (42 mg), and stirred another 2 h. The reaction mixture was concentrated, and the residue was purified by silica gel column chromatography using DCM/methanol (20:1) as the eluent. And to obtain compound 31 (2-((5-chloro-2-((3-chloro-4-(2-(cyclopentylamino)-7-azaspiro [3.5]nonan-7-yl)phenyl)amino)pyrimidin-4-yl)amino)phenyl) dimethyl-phosphine oxide (54.5 mg) as pale yellow solid. MS of Compound 31: 613[M+H]⁺.

¹H NMR (500 MHz, MeOD) δ 8.416 (s, 1H), 8.083 (s, 1H), 7.723 (s, 1H), 7.641-7.575 (m, 2H), 7.311-7.251 (m, 2H), 7.008-6.991 (m, 1H), 3.859-3.826 (m, 1H), 3.503-3.473 (m, 1H), 2.930-2.859(d, 4H), 2.405-2.366 (m, 2H), 2.124-2.076 (m, 2H), 1.978-1.940(m, 2H), 1.861-1.802(m, 12H), 1.707-1.591 (m, 4H).

### Example 32

### Synthesis of Compound 32

### (2-((5-chloro-2-((3-chloro-4-(2-((2-hydroxyethyl)amino)-7-azaspiro[3.5]nonan-7-yl)phenyl)amino)pyrimidin-4-yl)amino)phenyl)dimethylphosphine oxide

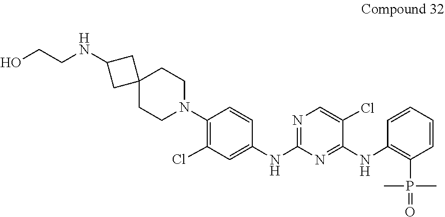 [SMILES: CP(C)(=O)c1ccccc1Nc1nc(Nc2ccc(N3CCC4(CC3)CC(NCCO)C4)c(Cl)c2)ncc1Cl]

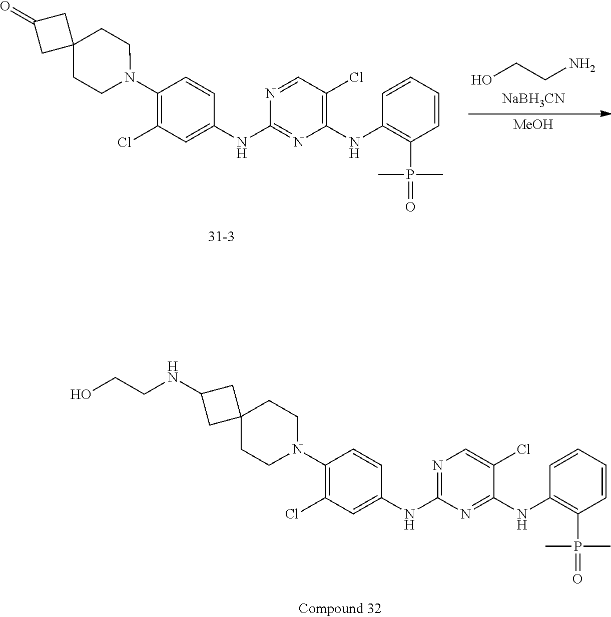

Following the same procedure as (2-((5-chloro-2-((3-chloro-4-(2-(cyclopentylamino)-7-azaspiro[3.5]nonan-7-yl)phenyl)amino)pyrimidin-4-yl)amino)phenyl)dimethylphosphine oxide using 2-aminoethan-1-ol instead of cyclopentanamine to obtain (2-((5-chloro-2-((3-chloro-4-(2-((2-hydroxyethyl)amino)-7-azaspiro[3.5]-nonan-7-yl)phenyl)amino)pyrimidin-4-yl)amino)phenyl)dimethyl-phosphine oxide. MS of Compound 32: 589 [M+H]⁺.

### Example 33

### Synthesis of Compound 33

### (2-((5-chloro-2-((3-chloro-4-(2-(pyrrolidin-1-yl)-7-azaspiro[3.5]nonan-7-yl)phenyl)amino)pyrimidin-4-yl)amino)phenyl)dimethylphosphine oxide

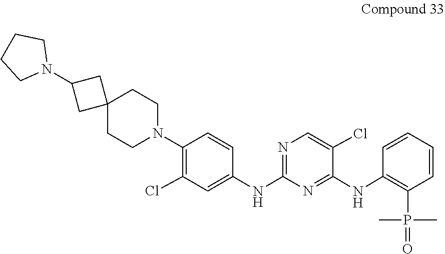 [SMILES: CP(C)(=O)c1ccccc1Nc1nc(Nc2ccc(N3CCC4(CC3)CC(N3CCCC3)C4)c(Cl)c2)ncc1Cl]

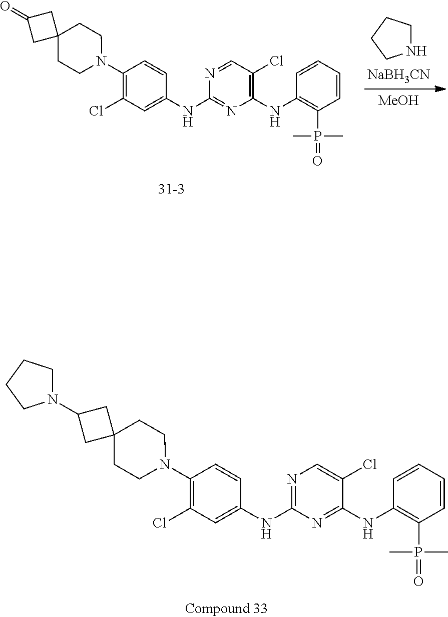

Following the same procedure as (2-((5-chloro-2-((3-chloro-4-(2-(cyclopentylamino)-7-azaspiro[3.5]nonan-7-yl)phenyl)amino)pyrimidin-4-yl)amino)phenyl)dimethylphosphine oxide using pyrrolidine instead of cyclopentanamine to obtain (2-((5-chloro-2-((3-chloro-4-(2-(pyrrolidin-1-yl)-7-azaspiro[3.5]nonan-7-yl)phenyl)amino)pyrimidin-4-yl)amino)phenyl)dimethylphosphine oxide. MS of Compound 33: 599 [M+H]⁺.

### Example 34

### Synthesis of Compound 34

### (R)-(2-((5-chloro-2-((3-chloro-4-(2-(3-(dimethylamino)pyrrolidin-1-yl)-7-azaspiro[3.5]nonan-7-yl)phenyl)amino)pyrimidin-4-yl)amino)phenyl)dimethylphosphine oxide

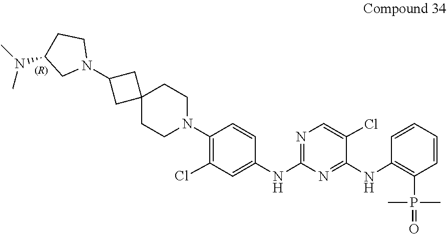

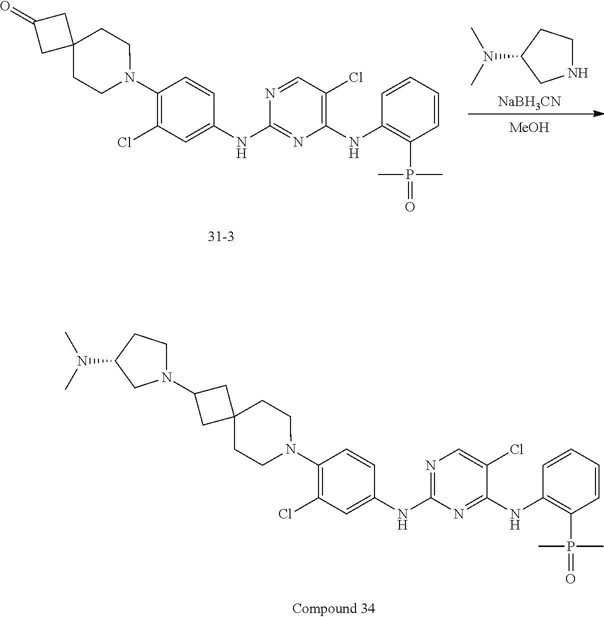

Following the same procedure as (2-((5-chloro-2-((3-chloro-4-(2-(cyclopentylamino)-7-azaspiro[3.5]nonan-7-yl)phenyl)amino)pyrimidin-4-yl)amino)phenyl)dimethylphosphine oxide using (R)-N,N-dimethylpyrrolidin-3-amine instead of cyclopentanamine to obtain compound 34 (R)-(2-((5-chloro-2-((3-chloro-4-(2-(3-(dimethylamino)pyrrolidin-1-yl)-7-azaspiro[3.5]nonan-7-yl)phenyl)amino)pyrimidin-4-yl)amino)phenyl)dimethyl-phosphine oxide. MS of Compound 34: 642 [M+H]⁺.

### Comparative Compound A

### (2-((5-chloro-2-((3-methyl-4-(2-methyl-2,7-diazaspiro[3.5]nonan-7-yl)phenyl)amino)pyrimidin-4-yl)amino)phenyl)dimethylphosphine oxide

Prepare the following Comparative compound A as described for Example 16 in WO2018108064.

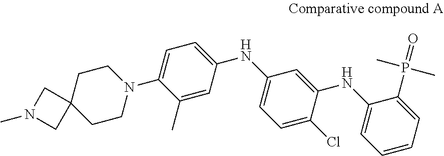

### Comparative Compound B

### (2-((5-chloro-2-((4-(6-ethyl-2,6-diazaspiro[3.3]heptan-2-yl)-3-methylphenyl)amino)pyrimidin-4-yl)amino)phenyl)dimethylphosphine oxide

Prepare the following Comparative compound B as described for Example 36 in WO2018108064.

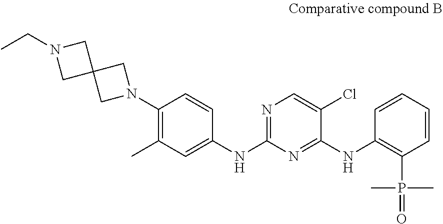 [SMILES: CCN1CC2(C1)CN(c1ccc(Nc3ncc(Cl)c(Nc4ccccc4P(C)(C)=O)n3)cc1C)C2]

### Pharmacological Testing

### Test 1 Kinase Assay for EGFR Del19/T790M/C797S and EGFR Wildtype

Mobility shift assay was performed to determine that the compounds exhibit affinity for EGFR Del19/T790M/C797S and EGFR kinase. Enzyme reaction protocol are as follows:

1. Preparing 1* Kinase buffer as followed.

| 1*kinase buffer | Final |
|---|---|
| HEPES PH7.5(mM) | 50 |
| Brij-35 | 0.0150% |
| DTT(mM) | 2 |
| Mgcl₂, Mncl₂ (mM) | 10 |

2. Preparing Compound Concentration Gradient: Compounds were tested at a concentration of 300 nM, diluted to 100-fold final concentration in 100% DMSO solution in 96-well plates, and compounds were diluted 3 times with Precision, 10 concentrations. Each concentration of the compound was then further diluted to a 5-fold final concentration of the intermediate dilution solution using 1* Kinase buffer.

3.5 μL of each of the prepared intermediate dilution compounds was separately added to the compound wells of the 384-well plate, and each concentration was tested for duplicate wells; 5 μL of 5% DMSO was added to the negative control wells and the positive control wells, respectively.

4. 2.5-fold final concentration of the kinase solution was prepared using 1*Kinase buffer.

5. Add 10 μL of 2.5-fold final concentration of kinase solution to the compound well and positive control well; add 10 μL of 1*Kinase buffer to the negative control well.

6. Centrifuge at 1000 rpm for 30 seconds, shake the reaction plate and incubate for 10 minutes at room temperature.

7. A mixed solution of 2.5 times the final concentration of ATP and Kinase substrate (5-FAM-EEPLYWSFPAKKK-CONH2) was prepared using 1*Kinase buffer.

8. 10 μL of a 2.5-fold final concentration of a mixed solution of ATP and a substrate was added to initiate the reaction.

9. Centrifuge the 384-well plate at 1000 rpm for 30 seconds, mix by shaking, and incubate at room temperature for the corresponding time.

10. Add 30 μL of the stop solution to stop the kinase reaction, centrifuge at 1000 rpm for 30 seconds, and mix by shaking.

11. Read the conversion rate with Caliper EZ Reader.

Convert conversion values to inhibition values:

Percent inhibition 32 (max-conversion)/(max-min)*100.

“max” stands for the mean value of the positive control well ratio; “min” stands for the mean value of the negative control well.

Fit the data in log(inhibitor) vs. response Variable slope of GraphPad Prism 5 to obtain IC50 values.

Equation used is: Y=Bottom+(Top-Bottom)/(1+(IC50/X)\hat{ }HillSlope)

The result is expressed with IC50, shown as Table 1, Compounds of the present disclosure, as exemplified in the Examples, showed IC₅₀ values in the following ranges: “*” stands for “IC₅₀≤2 nM”; “**” stands for “2 nM<IC₅₀≤10 nM”; “***” stands for IC₅₀>10 nM”.

TABLE 1

| | EGFR L858R/ | EGFR Del19/ | |
|---|---|---|---|
| EX | T790M/C797S | T790M/C797S | EGFR |
| No. | IC₅₀(nM) | IC₅₀(nM) | WT |
| 1 | * | 0.9 | 43 |
| 2 | ** | — | 66 |
| 3 | 2.8 | — | — |
| 4 | 1.5 | 1.0 | 21 |
| 5 | 1.5 | — | — |
| 6 | 1.3 | 0.37 | — |
| 7 | 7.8 | — | — |
| 8 | 1.6 | 0.58 | — |
| 9 | 11.0 | — | — |
| 10 | 0.7 | — | — |
| 11 | 2.6 | — | — |
| 12 | — | 1.0 | — |
| 13 | 1.3 | 0.6 | — |
| 14 | 1.2 | 0.31 | 27 |
| 15 | 0.7 | — | — |
| 16 | — | — | — |
| 17 | — | 0.7 | — |
| 18 | 16 | — | — |
| 19 | 4.5 | — | >300 |
| 20 | 2.3 | — | — |
| 21 | 0.9 | — | 20 |
| 22 | ** | — | 57 |
| 23 | 11.0 | — | 167 |
| 24 | 18.0 | — | 101 |
| 25 | 1.5 | — | 28 |
| 26 | 1.7 | — | 37 |
| 27 | 4.4 | — | 127 |
| 28 | *** | — | >300 |
| 29 | 1.6 | — | 35 |
| 30 | 1.1 | — | — |
| 31 | 1.6 | — | 33 |
| 32 | 3.2 | — | 65 |
| 33 | — | 0.4 | — |
| 34 | — | 0.53 | — |

Note:
“—” stands for “not tested”.

Test 2 Ba/F3-Del19/T790M/C797S and Ba/F3-L858R/T790M/C797S Cells Proliferation Assay

1. Cell Culture

Cell line: Ba/F3 cells with Del19/T790M/C797S or L858R/T790M/C797S mutation gene stably overexpressed named Ba/F3-Del19/T790M/C797S and Ba/F3-L858R/T790M/C797S.

A. Culture Medium

RPMI 1640 and 10% FBS and 1% PS.

B. Cell Recovery

a) The medium was preheated in a 37°0 C. water bath in advance.

b) Remove the cryogenic vials from the liquid nitrogen tank, quickly put it into a 37° C. water bath, and completely melt it in 1 min.

c) Transfer the cell suspension to a 15 mL centrifuge tube containing 8 mL medium, centrifuge 1000 rpm, 5 min.

d) Discard the supernatant, resuspend the cells in 1 mL medium, and transfer to a 75 cm² flask containing 15 mL medium, culture the cells in an incubator at 37° C., 5% CO₂.

C. Cell Passage

a) The medium was preheated in a 37° C. water bath in advance.

b) Collect cell to a 15 mL centrifuged tube, centrifuge at 1000 rpm for 5 min. Discard the supernatant, count, and make the cell density at 1×10⁴ cells/mL, then place it in a 37° C., 5% CO₂ incubator.

2. Compound Preparation

a) The test compound (20 mM stock solution) was diluted to 200uM with 100% DMSO as starting concentration then 3-fold serial diluted with “9+0” concentrations. in a 96-well dilution plate (Cat # P-05525, Labcyte);

b) The above compound solution was diluted 1:20 times with culture medium to prepare a 10 fold working solution;

3. Cell Plating

a) Take cells in log phase growth, centrifuge at 1000 rpm for 5 min, then resuspend the cells with culture medium, then count cells;

b) Cells were seeded to 96-well cell culture plate with density at 2000 cells/well;

4. Compound Treatment

a) Compounds prepared at step 2 were added to cell plate with 15 μL per well, the final concentrations were 1000,333, 111.1, 37,12.3, 4.1, 1.4, 0.5, 0.2 and 0 nM, and the final concentration of DMSO was 0.5%. The blank control well was a culture medium (0.5% DMSO);

b) The cells were incubated for an additional 72 h in the incubator.

5. Detection

a) Remove the 96-well cell culture plate and add 50 μl of CTG reagent (CellTiter Glo kit, promega, Cat # G7573).

b) Plate was shaked for 2 min and then let it cool for 10 min at room temperature.

c) The Luminescence signal value was read using a PerkinElmer reader. Experimental data analysis

Data were analyzed using GraphPad Prism 6.0 software to obtain a fitted curve of compound activity.

Fit the Compound IC50 from non-linear regression equation:

Y=Bottom+(Top-Bottom)/(1+10((LogIC50-X)×Hill Slope));

X: The log of the concentration of the compound; Y: Luminescence value.

The cells proliferation assay results are expressed with IC₅₀, shown as Table 2. Compounds of the present disclosure, as exemplified in the Examples, showed IC₅₀ values in the following ranges: “*” stands for “IC₅₀≤10 nM”; “**” stands for “10 nM<IC₅₀≤50 nM”; “***” stands for “IC₅₀>50 nM”.

TABLE 2

| EX | Ba/F3-Del19/T790M/C797S | Ba/F3-L858R/T790M/C797S |
|---|---|---|
| No. | IC₅₀(nM) | IC₅₀ (nM) |
| 1 | 5.8 | 6.0 |
| 2 | ** | ** |
| 3 | ** | ** |
| 4 | * | ** |
| 5 | ** | ** |
| 6 | ** | ** |
| 7 | *** | *** |
| 8 | 21.5 | 17.3 |
| 9 | *** | *** |
| 10 | 10.2 | 24.7 |
| 11 | 26.3 | ** |
| 12 | 24.4 | ** |
| 13 | * | * |
| 14 | 13.2 | 15.1 |
| 15 | 15.5 | 17.0 |
| 16 | 8.0 | 6.9 |
| 17 | * | * |
| 18 | ** | ** |
| 19 | ** | ** |
| 20 | ** | ** |
| 21 | *** | *** |
| 22 | *** | *** |
| 23 | *** | *** |
| 24 | *** | *** |
| 25 | 18.0 | 24.2 |
| 26 | ** | ** |
| 27 | *** | *** |
| 28 | *** | *** |
| 29 | 16.9 | 32.9 |
| 30 | * | ** |
| 31 | ** | ** |
| 32 | *** | *** |
| 33 | 9.7 | 11.7 |
| 34 | 14.7 | 21.7 |

Test 4 A431 Cells Proliferation Assay

1. Cell Culture

Cell line: A431 (WT)

A. Culture Medium

DMEM and 10% FBS and 1% PS.

B. Cell Recovery

a) The medium was preheated in a 37° C. water bath in advance.

b) Remove the cryogenic vials from the liquid nitrogen tank, quickly put it into a 37° C. water bath, and completely melt it in 1 min.

c) Transfer the cell suspension to a 15 mL centrifuge tube containing 8 mL medium, centrifuge 1000 rpm, 5 min.

d) Discard the supernatant, resuspend the cells in 1 mL medium, and transfer to a 75 cm² flask containing 15 mL medium, culture the cells in an incubator at 37° C., 5% CO₂.

C. Cell Passage

a) The medium was preheated in a 37° C. water bath in advance.

b) Collect cell to a 15 mL centrifuged tube, centrifuge at 1000 rpm for 5 min. Discard the supernatant, count, and make the cell density at 1×10⁴ cells/mL, then place it in a 37° C., 5% CO₂ incubator.

2. Compound Preparation

a) The test compound (20 mM stock solution) was diluted to 2 mM with 100% DMSO as starting concentration then 3-fold serial diluted with “9+0” concentrations. in a 96-well dilution plate (Cat # P-05525, Labcyte);

b) The above compound solution was diluted 1:40 times with culture medium to prepare a 5 fold working solution;

3. Cell Plating

a) Take cells in log phase growth, centrifuge at 1000 rpm for 5 min, then resuspend the cells with culture medium, then count cells;

b) Cells were seeded to 96-well cell culture plate with density at 5000 cells/well;

4. Compound Treatment

a) Compounds prepared at step 2 were added to cell plate with 40 μL per well, the final concentrations were 10000, 3333,1111.1, 370.4, 123.5, 41.2, 13.7, 4.6, 1.5 and 0 nM, and the final concentration of DMSO was 0.5%. The blank control well was a culture medium (0.5% DMSO);

b) The cells were incubated for an additional 72 h in the incubator.

5. Detection

a) Remove the 96-well cell culture plate and add 60 μl of CTG reagent (CellTiter Glo kit, promega, Cat # G7573).

b) Plate was shaked for 2 min and then let it cool for 10 min at room temperature.

c) The Luminescence signal value was read using a PerkinElmer reader.

Experimental Data Analysis

Data were analyzed using GraphPad Prism 6.0 software to obtain a fitted curve of compound activity.

Fit the Compound IC50 from non-linear regression equation:

Y=Bottom+(Top-Bottom)/(1+10((LogIC50-X)×HillSlope));

X: The log of the concentration of the compound; Y: Luminescence value.

The cells proliferation assay results are expressed with IC₅₀, shown as Table 3. Compounds of the present disclosure, as exemplified in the Examples, showed IC₅₀ values in the following ranges: “*” stands for “IC₅₀≤10 nM”; “**” stands for “10 nM<IC₅₀≤50 nM”; “***” stands for “IC₅₀>50 nM”.

TABLE 3

| EX No. | A431 (WT) IC₅₀ (nM) |
|---|---|
| 1 | 48.8 |
| 2 | — |
| 3 | — |
| 4 | 26.9 |
| 5 | — |
| 6 | 44.7 |
| 7 | 341.1 |
| 8 | 57.5 |
| 9 | — |
| 10 | 41.6 |
| 11 | — |
| 12 | 121.0 |
| 13 | 41.6 |
| 14 | 17.5 |
| 15 | 27.6 |
| 16 | — |
| 17 | 35 |
| 18 | — |
| 19 | 349 |
| 20 | — |
| 21 | 367 |
| 22 | — |
| 23 | — |
| 24 | — |
| 25 | — |
| 26 | — |
| 27 | — |
| 28 | — |
| 29 | 95.5 |
| 30 | 30.0 |
| 31 | — |
| 32 | — |
| 33 | 47.0 |
| 34 | 53 |

Note:
“—” stands for “not tested”.

### Test 5 Pharmacokinetic Assay

Male SD rats, oral administration (intragastric administration), 3 in each group. Animals administered by gavage were fasted overnight before the experiment, and the fasting time was from at least 12 hours before administration to 4 hours after administration. The blood was collected using the orbital vein. Time of blood collection by oral administration: 15 min, 30 min, 1 h, 2 h, 4 h, 7 h, 24 h. The blood collection volume was 300 uL, and after anticoagulation with 2.0% EDTA, the blood was centrifuged at 4000 rpm for 5 min, and the blood plasma was taken for about 100 uL, and placed in −20° C. for examination. The plasma sample was analyzed using liquid chromatography tandem mass spectrometry (LC-MS/MS). Plasma concentration-time data for individual animals were analyzed using WinNonlin (V4.1, Pharsight) software with a non-compartmental model and the pharmacokinetic parameters of the test compounds were calculated. PK properties of the compounds in rats is shown in Table 4.

TABLE 4

| | | Administration | | t_{1/2} | tₘₐₓ | Cₘₐₓ | AUC₀₋ₜ |
|---|---|---|---|---|---|---|---|
| Compound | Formulation | method | | (h) | (h) | (ng/mL) | (h · ng/mL) |
| Comparative | 5% DMSO/5% Solutol/90% | p.o.10 | mg/kg | 5.43 | 5.0 | 287 | 3383 |
| compound A | normal saline | | | | | | |
| Comparative | 5% DMSO/5% Solutol/90% | p.o.5 | mg/kg | 4.09 | 2.75 | 67.2 | 202 |
| compound B | normal saline | | | | | | |
| 1 | 5% DMSO/5% Solutol/90% | p.o.10 | mg/kg | 6.53 | 1.67 | 141 | 1544 |
| | normal saline | | | | | | |
| 4 | 10% DMSO/10% Solutol/80% | p.o.5 | mg/kg | 6.27 | 6.00 | 71.3 | 1017 |
| | normal saline | | | | | | |
| 6 | 10% DMSO/10% Solutol/80% | p.o.5 | mg/kg | 3.00 | 4.00 | 741 | 8807 |
| | normal saline | | | | | | |
| 8 | 5% DMSO/5% Solutol/90% | p.o.5 | mg/kg | 3.17 | 2.08 | 388 | 2817 |
| | normal saline | | | | | | |
| 10 | 20%Solutol/80% normal | p.o.5 | mg/kg | 9.06 | 4.0 | 35.1 | 443 |
| | saline | | | | | | |
| 15 | Saline/Solutol(90/10, v/v)) | p.o.5 | mg/kg | 4.38 | 6.0 | 575 | 6936 |

1. A compound of Formula I, or a stereoisomer, tautomer, pharmaceutically acceptable salt, prodrug, chelate, non-covalent complex, or solvate thereof, 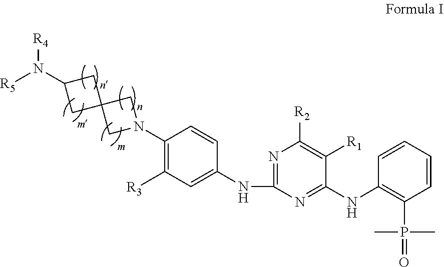
wherein,
R₁ is H, CN, halogen, —C₁₋₆ alkyl or C₁₋₆ alkoxyl;
R₂ is H, halogen, or —C₁₋₆ alkyl; or
R₁ and R₂ together with the atoms to which they are attached form a 5- to 6-membered heteroaryl ring optionally comprising 1, 2 or 3 hetero atoms independently selected from N, S, or O;
R₃ is H, halogen, —C₁₋₆ alkyl, —C₁₋₄ haloalkyl, —C₃₋₆ carbocyclic ring;
R₄ and R₅ are each independently selected from H, —C₁₋₆ alkyl, —C₁₋₄ alkyl-OH, or —C₃₋₆ carbocyclic ring; or
R₄ and R₅ together with the atoms to which they are attached form a 5- to 6-membered heterocyclic ring optionally substituted with one or more substituents independently selected from —C₁₋₆ alkyl, halogen, or —NR₆R₇;
R₆ and R₇ are each independently selected from H, or —C₁₋₆ alkyl;
m, n, m′, and n′ are each independently selected from 1 or 2.

2. The compound of claim 1, wherein Ri is independently selected from H, F, Cl, CH₃, —OCH₃ or CN.

3. The compound of claim 1, wherein R₂ is H.

4. The compound of claim 1, wherein R₁ and R₂ together with the atoms to which they are attached form

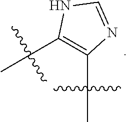 [SMILES: *c1nc[nH]c1*]

5. The compound of claim 1-4, wherein R₃ is independently selected from H, —CH₃, 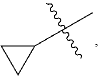 [SMILES: *C1CC1]
Cl, F, and CF₃.

6. The compound of claim 1, wherein R₄ and R₅ are independently selected from H, —CH₃, —CH₂CH₂OH,

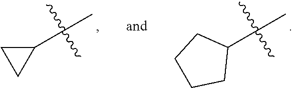

7. The compound of claim 1, wherein R₄ and R₅ are both —CH₃.

8. The compound of of claim 1, wherein R₄ and R₅ together with the atoms to which they are attached form

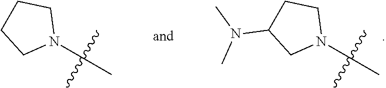

9. The compound of claim 1, wherein the compound is
1) (2-((5-chloro-2-((4-(7-(dimethyl amino)-2-azaspiro[3.5]nonan-2-yl)-3-methylphenyl)amino)pyrimidin-4-yl)amino)phenyl)dimethylphosphine oxide;
2) (2-((5-chloro-2-((3-methyl-4-(7-(methylamino)-2-azaspiro[3. ]nonan-2-yl)phenyl)amino)pyrimidin-4-yl)amino)phenyl)dimethylphosphine oxide;
3) (2-((2-((4-(7-amino-2-azaspiro[3.5]nonan-2-yl)-3-methylphenyl)amino)-5-chloropyrimidin-4-yl) amino)phenyl)dimethylphosphine oxide;
4) (2-((5-chloro-((4-(7-(cyclopropylamino)-2-azaspiro[3.5]nonan-2-yl)-3-methylphenyl)amino)pyrimidin-4-yl)amino)phenyl)dimethylphosphine oxide;
5) (2-((5-chloro-2-((3-cyclopropyl-4-(7-(dimethylamino)-2-azaspiro[3.5]nonan-2-yl)phenyl)amino) pyrimidin-4-yl)amino)phenyl)dimethylphosphine oxide;
6) (2-((5-chloro-2-((3-chloro-4-(2-(dimethylamino)-7-azaspiro[3.5]nonan-7-yl)phenyl)amino)pyrmidin-4-yl)amino)phenyl)dimethylphosphine oxide;
7) (2-((2-((3-chloro-4-(2-(dimethylamino)-7-azaspiro[3.5]nonan-7-yl)phenyl)amino)pyrimidin-4-yl)amino)phenyl)dimethylphosphine oxide;
8) (2-((2-((4-(2-amino-7-azaspiro[3.5]nonan-7-yl)-3-chlorophenyl)amino)-5-chloropyrimidin-4-yl) amino)phenyl)dimethylphosphine oxide;
9) (2-((2-((4-(2-amino-7-azaspiro[3.5]nonan-7-yl)-3-chlorophenyl)amino)pyrimidin-4-yl)amino)phenyl)dimethylphosphine oxide;
10) (2-((5-chloro-2-((3-chloro-4-(7-(dimethylamino)-2-azaspiro[3.5]nonan-2-yl)phenyl)amino)pyrmidin-4-yl)amino)phenyl)dimethylphosphine oxide;
11) (2-((2-((4-(7-amino-2-azaspiro[3.5]nonan-2-yl)-3-chlorophenyl)amino)-5-chloropyrimidin-4-yl) amino)phenyl)dimethylphosphine oxide;
12) (2-((5-chloro-2-((3-chloro-4-(742-hydroxyethyl)amino)-2-azaspiro[3.5]nonan-2-yl)phenyl)amino)pyrimidin-4-yl)amino)phenyl)dimethylphosphine oxide;
13) (2-((5-chloro-2-((4-(7-(dimethylamino)-2-azaspiro[3.5]nonan-2-yl)phenyl)amino)pyrimidin-4-yl)amino)phenyl)dimethylphosphine oxide;
14) (2-((2-((4-(2-amino-7-azaspiro[3.5]nonan-7-yl)-3-methylphenyl)amino)-5-chloropyrimidin-4-yl) amino)phenyl)dimethylphosphine oxide;
15) (2-((5-chloro-2-((4-(2-(dimethylamino)-7-azaspiro[3.5]nonan-7-yl)-3-methylphenyl)amino)pyrmidin-4-yl)amino)phenyl)dimethylphosphine oxide;
16) (2-((2-((4-(2-amino-7-azaspiro[3.5]nonan-7-yl)phenyl)amino)-5-chloropyrimidin-4-yl)amino)phenyl)dimethylphosphine oxide hydrochloride;
17) (2-((5-chloro-2-((4-(2-(dimethylamino)-7-azaspiro[3.5]nonan-7-yl)phenyl)amino)pyrimidin-4-yl)amino)phenyl)dimethylphosphine oxide;
18) (2-((2-((4-(2-amino-7-azaspiro[3.5]nonan-7-yl)-3-(trifluoromethyl)phenyl)amino)-5-chloropyrmidin-4-yl)amino)phenyl)dimethylphosphine oxide;
19) (2-((5-chloro-2-((4-(2-(dimethylamino)-7-azaspiro[3.5]nonan-7-yl)-3-(trifluoromethyl)phenyl)amino)pyrimidin-4-yl)amino)phenyl)dimethylphosphine oxide;
20) (2-((5-chloro-2-((3-chloro-4-(2-(methylamino)-7-azaspiro[3.5]nonan-7-yl)phenyl)amino)pyrimdin-4-yl)amino)phenyl)dimethylphosphine oxide;
21) (2-((2-((4-(2-amino-7-azaspiro[3.5]nonan-7-yl)-3-methylphenyl)amino)-9H-purin-6-yl)amino)phenyl)dimethylphosphine oxide;
22) (2-((2-((4-(6-amino-2-azaspiro[3.3]heptan-2-yl)-3-methylphenyl)amino)-5-methylpyrimidin-4-yl)amino)phenyl)dimethylphosphine oxide;
23) (2-((2-((4-(6-amino-2-azaspiro[3.3]heptan-2-yl)-3-methylphenyl)amino)-5-methoxypyrimidin-4 -yl)amino)phenyl)dimethylphosphine oxide;
24) (2-((2-((4-(6-amino-2-azaspiro[3.3]heptan-2-yl)-3-methylphenyl)amino)-5-fluoropyrimidin-4-yl) amino)phenyl)dimethylphosphine oxide;
25) (2-((2-((4-(6-amino-2-azaspiro[3.3]heptan-2-yl)-3-methylphenyl)amino)-5-chloropyrimidin-4-yl)amino)phenyl)dimethylphosphine oxide;
26) (2-((2-((4-(6-amino-2-azaspiro[3.3]heptan-2-yl)-3-chlorophenyl)amino)-5-chloropyrimidin-4-yl) amino)phenyl)dimethylphosphine oxide;
27) (2-((2-((4-(2-amino-7-azaspiro[3 5]nonan-7-yl)-3-methylphenyl)amino)-5-methoxypyrimidin-4-yl)amino)phenyl)dimethylphosphine oxide;
28) 2-((4-(2-amino-7-azaspiro[3.5]nonan-7-yl)-3-methylphenyl)amino)-4-((2-(dimethylphosphoryl) phenyl)amino)pyrimidine-5-carbonitrile;
29) (2-((2-((4-(2-amino-7-azaspiro[3.5]nonan-7-yl)-3-fluorophenyl)amino)-5-chloropyrimidin-4-yl) amino)phenyl)dimethylphosphine oxide;
30) (2-((5-chloro-2-((4-(2-(dimethylamino)-7-azaspiro[3.5]nonan-7-yl)-3-fluorophenyl)amino)pyrimidin-4-yl)amino)phenyl)dimethylphosphine oxide;
31) (2-((5-chloro-2-((3-chloro-4-(2-(cyclopentylamino)-7-azaspiro[3.5]nonan-7-yl)phenyl)amino)pyrimidin-4-yl)amino)phenyl)dimethylphosphine oxide;
32) (2-((5-chloro-2-((3-chloro-4-(2-((2-hydroxyethyl)amino)-7-azaspiro[3.5]nonan-7-yl)phenyl)amino)pyrimidin-4-yl)amino)phenyl)dimethylphosphine oxide;
33) (2-((5-chloro-2-((3-chloro-4-(2-(pyrrolidin-1-yl)-7-azaspiro[3.5]nonan-7-yl)phenyl)amino)pyrmidin-4-yl)amino)phenyl)dimethylphosphine oxide; or
34) (R)-(2-((5-chloro-2-((3-chloro-4-(2-(3-(dimethylamino)pyrrolidin-1-yl)-7-azaspiro[3.5]nonan-7-yl)phenyl)amino)pyrimidin-4-yl)amino)phenyl)dimethylphosphine oxide.

10. A pharmaceutical composition comprising a compound of claim 1, or a pharmaceutically acceptable salt or a stereoisomer thereof, and at least one pharmaceutically acceptable carrier or excipient.

11. A method of inhibiting mutant EGFR, including but not limited to EGFR C797S, said method comprising administering to a patient a compound of claim 1, or a pharmaceutically acceptable salt or a stereoisomer thereof.

12. A method of treating an EGFR-driven cancer, said method comprising administering to a patient in need thereof a therapeutically effective amount of a compound of claim 1, or a pharmaceutically acceptable salt or a stereoisomer thereof.

13. The method of claim 12, wherein the EGFR-driven cancer is characterized by the presence of one or more mutations selected from, but not limited to (i) C797S, (ii) both L858R and C797S, (iii) both C797S and T790M, (iv) L858R, T790M, and C797S, or (v) De119, T790M and C797S.

14. The method of claim 12, wherein the EGFR-driven cancer is colon cancer, gastric cancer, thyroid cancer, lung cancer, leukemia, pancreatic cancer, melanoma, multiple melanoma, brain cancer, renal cancer, prostate cancer, ovarian cancer or breast cancer.

15. The method of claim 12, wherein the EGFR-driven cancer is non-small-cell lung cancer.

16-20. (canceled)
